(19) United States
(12) Patent Application Publication
(10) Pub. No.: US 2026/0297016 A1
(43) Pub. Date: Oct. 1, 2026

(54) THERAPEUTIC COMPOUNDS

(71) Applicants: REGENTS OF THE UNIVERSITY OF MINNESOTA, Minneapolis, MN (US); THE BOARD OF REGENTS OF THE UNIVERSITY OF OKLAHOMA, Norman, OK (US); WAKE FOREST UNIVERSITY HEALTH SCIENCES, Winston-Salem, NC (US)

(72) Inventors: Adam Duerfeldt, Minneapolis, MN (US); Julia Lee, Minneapolis, MN (US); Ziwei Hu, Minneapolis, MN (US); Dinesh Nath, Norman, OK (US); Jian-Xing Ma, Winston-Salem, NC (US); Bo HU, Minneapolis, MN (US)

(73) Assignees: REGENTS OF THE UNIVERSITY OF MINNESOTA, Minneapolis, MN (US); THE BOARD OF REGENTS OF THE UNIVERSITY OF OKLAHOMA, Norman, OK (US); WAKE FOREST UNIVERSITY HEALTH SCIENCES, Winston-Salem, NC (US)

(21) Appl. No.: 19/478,203

(22) PCT Filed: Apr. 24, 2024
(86) PCT No.: PCT/US2024/025990

§ 371 Date: Oct. 23, 2025

Related U.S. Application Data
(60) Provisional application No. 63/461,433, filed on Apr. 24, 2023.

(51) Int. Cl. C07C 229/60; A61K 31/196; A61K 31/277; A61K 31/41; A61K 31/4192; A61K 31/426; A61K 31/4418; C07C 229/64; C07C 255/58; C07C 255/59; C07C 317/32; C07D 213/38; C07D 249/06; C07D 257/04; C07D 277/28

(52) U.S. Cl. CPC C07C 229/60; A61K 31/196; A61K 31/277; A61K 31/41; A61K 31/4192; A61K 31/426; A61K 31/4418; C07C 229/64; C07C 255/58; C07C 255/59; C07C 317/32; C07D 213/38; C07D 249/06; C07D 257/04; C07D 277/28; C07C 2601/08

(57) ABSTRACT

The invention provides a compound of formula (I): or a salt thereof, wherein A, B, C, R¹, and R have any of the values described in the specification, as well as compositions comprising a compound of formula (I). The compounds are useful for agonizing the activity of PPARα and for treating diseases modulated by PPARα, such as, for example, diabetic retinopathy, pain, and liver diseases including fibrosis, NASH and NAFLD. Certain compounds are also useful as both agonists of PPARα and inhibitors of STING.

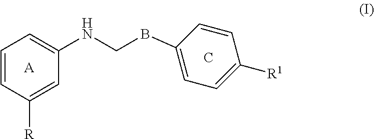

### CROSS REFERENCE TO RELATED APPLICATION

This application claims priority from U.S. Provisional Patent Application No. 63/461,433, filed 24 Apr. 2023. The entire content of U.S. Provisional Patent Application No. 63/461,433 is hereby incorporated by reference herein.

### STATEMENT REGARDING FEDERALLY SPONSORED RESEARCH

This invention was made with government support under EY030472, EY028949, EY034510, and EY033330 awarded by National Institutes of Health. The government has certain rights in the invention.

### BACKGROUND

Diabetic retinopathy (DR) is a common microvascular complication of diabetes and the leading cause of blindness in the working-age population (Nentwich, M. M. & Ulbig, M. W., World J. Diabetes 6, 489-499 (2015)). Although several treatment options are available (Gregori, N. Z, Ophthalmology www.aao.org/eye-health/diseases/what-is-diabetic-retinopathy (2021)), addressing the complex nature of DR remains a significant challenge (Cai, X. & McGinnis, J. F., J. Diabetes Res. 2016, U.S. Pat. No. 3,789,217 (2016)). Direct intraocular injection of anti-VEGF antibodies has emerged as the standard of care; however, this treatment suffers from the requirement of recurrent injections and ~40% of patients fail to respond to this therapy (Simó, R. & Hernandez, C., Prog. Retin. Eye Res. 48, 160-180 (2015)). A large part of unresponsiveness is likely due to disease heterogeneity, with other pathological features of DR (e.g., inflammation, fibrosis, ischemia, vascular leakage) being the driving force(s) for disease in patient subsets, rather than VEGF-mediated processes (e.g., neovascularization) (Sapieha, P. et al., Int. J. Biochem. Cell Biol. 42, 5-12 (2010)). New therapies that are superior or complementary to current approaches would be valuable to patients, especially for those that remain refractory to anti-VEGF options. Ideally, unlike current treatment choices, new approaches would be noninvasive (to the eye), address other pathological drivers, and noncontingent on specialized facilities.

One target that is gaining popularity for DR is peroxisome proliferator-activated receptor alpha (PPARα). PPARα is a ligand-activated transcription factor with known endogenous (e.g., fatty acids) and synthetic ligands (Berger, J. & Moller, D. E., Annu. Rev. Med. 53, 409-435 (2002)). Two additional PPAR members (PPARγ and PPARβ/δ) exist, and each PPAR subtype exhibits a unique tissue distribution, maintains diverse functions, and can be selectively targeted (Kliewer, S. A. et al., Proc. Natl. Acad Sci. U.S.A. 91, 7355-7359 (1994)). Specifically, it has been shown that PPARα is expressed at high levels in all types of retinal cells and plays fundamental roles not only in regulating VEGF but also in mitochondrial function, inflammation, apoptosis, and angiogenesis in the retina (Hu, Y. et al., Proc. Natl. Acad Sci. U.S.A. 110, 15401-15406 (2013)). PPARα levels in the retina are significantly decreased in diabetic animal models and humans, and PPARα ablation results in retinal mitochondrial dysfunction, a declined electroretinogram (ERG) response, and retinal cell apoptosis (Hu, Y. et al., Proc. Natl. Acad Sci. U.S.A. 110, 15401-15406 (2013); Pearsall, E. A. et al., BMC Biol. 15, 113 (2017); and Chen, Y. et al., Diabetes 62, 261-272 (2013)).

Two large, prospective, independent clinical trials (FIELD and ACCORD) have demonstrated that fenofibrate, an orally administered medication used to treat dyslipidemia, significantly reduced the progression of DR and other microvascular endpoints in patients with type 2 diabetes (ACCORD Study Group et al., N. Engl. J. Med 363, 233-244 (2010); and Keech, A. C. et al., Lancet 370, 1687-1697 (2007)). The therapeutic benefits are not related to its lipid-lowering activity, but rather result from the agonism of PPARα by the major metabolite, fenofibric acid (FA) (Chen, Y. et al., Diabetes 62, 261-272 (2013); and Noonan, J. E. et al., Diabetes 62, 3968-3975 (2013).). Fenofibrate, however, suffers from a low affinity for PPARα, lack of selectivity among PPAR subtypes, and dose-limiting toxicities, all of which will likely limit its use as a DR therapy (Li, J. et al., J. Med. Chem. 53, 2854-2864 (2010)).

Currently there is a need for additional agonists of PPARα. Such compounds would be useful for treating PPARα related pathologies, such as, for example, DR.

### SUMMARY

In one aspect the present invention provides a compound of formula (I):

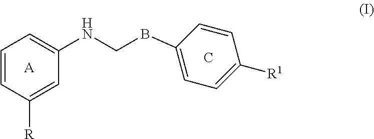

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
B is phenyl, a 5-membered heteroaryl, or a 6-membered heteroaryl;
R¹ is H, halo, cyano, (C₁-C₄)alkyl, (C₃-C₆)cycloalkyl, (C₁-C₄)alkylsulfonyl, hydroxy, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, N^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₁-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
   each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

The invention also provides a pharmaceutical composition comprising a compound of formula (I) or a pharmaceutically acceptable salt thereof and a pharmaceutically acceptable carrier.

The invention also provides, a method for treating diabetic retinopathy in an animal, comprising administering to the animal, a compound of formula (I) or a pharmaceutically acceptable salt thereof.

The invention also provides a method for agonizing the activity of a peroxisome proliferator-activated receptor alpha comprising contacting the receptor with a compound of formula (I) or a salt thereof in vitro or in vivo.

The invention also provides a method for agonizing the activity of a peroxisome proliferator-activated receptor alpha in an animal comprising administering to the animal, a compound of formula (I) or a pharmaceutically acceptable salt thereof.

The invention also provides a method for treating pain in an animal comprising administering to the animal, a compound of formula (I) as described in any one of claims 1-19 or a pharmaceutically acceptable salt thereof.

The invention also provides a method for treating a disease or condition associated with the liver in an animal (e.g., fibrosis, nonalcoholic fatty liver disease (NAFLD or metabolic dysfunctio-associated steatotic liver disease MASLD)), or nonalcoholic steatohepatitis (NASH)), comprising administering to the animal, a compound of formula (I) as described in any one of claims 1-19 or a pharmaceutically acceptable salt thereof.

The invention also provides, a method for treating a disease or condition in an animal comprising administering a compound of formula (I) or a pharmaceutically acceptable salt thereof to the animal, wherein the disease or condition is selected from the group consisting of: systemic diseases with an obvious inflammatory basis, inflammatory bowel disease, type 1 diabetes, Graves disease, multiple sclerosis, various types of arthritis, vasculitis, dermatitis, glomerulonephritis, hepatitis, periodonititis; and non-obvious inflammatory systemic disease with an inflammatory basis including, diabetes, atherosclerosis, heart failure, obesity, Alzheimer's disease, and metabolic syndrome.

The invention also provides, a method for treating a disease or condition in an animal comprising administering a compound of formula (I) or a pharmaceutically acceptable salt thereof to the animal, wherein the disease or condition is selected from the group consisting of: ocular disease with an obvious inflammatory basis, including keratitis, endophthalmitis, blepharitis, conjunctivitis, scleritis, herpetic inflammation, uveitis, vasculitis, arteritis, orbital inflammations, optic neuritis, sympathetic ophthalmia, retinitis, and other autoimmune diseases; and non-obvious inflammatory ocular diseases with an inflammatory basis including, age-related macular degeneration, macular edema, diabetic retinopathy, glaucoma, proliferative vitreoretinopathy, and corneal, uveal, or retinal edema.

The invention also provides, a method for treating a disease or condition in an animal comprising administering a compound of formula (I) or a pharmaceutically acceptable salt thereof to the animal, wherein the disease or condition is selected from the group consisting of: conditions and/or disorders characterized by angiogenesis, including ocular disease with an obvious angiogenesis basis, including diabetic retinopathy, age-related macular degeneration, retinopathy of prematurity, retinal artery or vein occlusion, corneal graft rejection, corneal neovascularization, neovascular glaucoma and sickle cell retinopathy; and non-ocular disease with an obvious angiogenesis basis, including, cancer, skin diseases, diabetic ulcers, diabetic nephropathy, cardiovascular disease and stroke.

The invention also provides, a compound of formula (I) or a pharmaceutically acceptable salt thereof for use in medical therapy.

The invention also provides, a compound of formula (I) or a pharmaceutically acceptable salt thereof for the prophylactic or therapeutic treatment of diabetic retinopathy.

The invention also provides a compound of formula (I) or a pharmaceutically acceptable salt thereof for agonizing the activity of a peroxisome proliferator-activated receptor alpha.

The invention also provides, the use of a compound of formula (I) or a pharmaceutically acceptable salt thereof to prepare a medicament for treating diabetic retinopathy in an animal.

The invention also provides a compound of formula (I) or a pharmaceutically acceptable salt thereof for the prophylactic or therapeutic treatment of pain.

The invention also provides a compound of formula (I) or a pharmaceutically acceptable salt thereof for the prophylactic or therapeutic treatment of a disease or condition associated with the liver (e.g., fibrosis, nonalcoholic fatty liver disease (NAFLD or metabolic dysfunctio-associated steatotic liver disease MASLD), or nonalcoholic steatohepatitis (NASH)).

The invention also provides, a compound of formula (I) or a pharmaceutically acceptable salt thereof for the treatment of a disease or condition, wherein the disease or condition is selected from the group consisting of: systemic diseases with an obvious inflammatory basis, inflammatory bowel disease, type 1 diabetes, Graves disease, multiple sclerosis, various types of arthritis, vasculitis, dermatitis, glomerulonephritis, hepatitis, periodonititis; and non-obvious inflammatory systemic disease with an inflammatory basis including, diabetes, atherosclerosis, heart failure, obesity, Alzheimer's disease, and metabolic syndrome.

The invention also provides, a compound of formula (I) or a pharmaceutically acceptable salt thereof for the treatment of a disease or condition, wherein the disease or condition is selected from the group consisting of: ocular disease with an obvious inflammatory basis, including keratitis, endophthalmitis, blepharitis, conjunctivitis, scleritis, herpetic inflammation, uveitis, vasculitis, arteritis, orbital inflammations, optic neuritis, sympathetic ophthalmia, retinitis, and other autoimmune diseases; and non-obvious inflammatory ocular diseases with an inflammatory basis including, age-related macular degeneration, macular edema, diabetic retinopathy, glaucoma, proliferative vitreoretinopathy, and corneal, uveal, or retinal edema.

The invention also provides, a compound of formula (I) or a pharmaceutically acceptable salt thereof for the treatment of a disease or condition, wherein the disease or condition is selected from the group consisting of: conditions and/or disorders characterized by angiogenesis, including ocular disease with an obvious angiogenesis basis, including diabetic retinopathy, age-related macular degeneration, retinopathy of prematurity, retinal artery or vein occlusion, corneal graft rejection, corneal neovascularization, neovascular glaucoma and sickle cell retinopathy; and non-ocular disease with an obvious angiogenesis basis, including, cancer, skin diseases, diabetic ulcers, diabetic nephropathy, cardiovascular disease and stroke.

The invention also provides, the use of a compound of formula (I) or a pharmaceutically acceptable salt thereof to prepare a medicament for treating diabetic retinopathy in an animal.

The invention also provides, the use of a compound of formula (I) or a pharmaceutically acceptable salt thereof to prepare a medicament for agonizing the activity of a peroxisome proliferator-activated receptor alpha in an animal.

The invention also provides, the use of a compound of formula (I) or a pharmaceutically acceptable salt thereof to prepare a medicament for treating pain in an animal.

The invention also provides, the use of a compound of formula (I) or a pharmaceutically acceptable salt thereof to prepare a medicament for treating a disease or condition associated with the liver (e.g., fibrosis, nonalcoholic fatty liver disease (NAFLD or metabolic dysfunctio-associated steatotic liver disease MASLD), or nonalcoholic steatohepatitis (NASH)) in an animal.

The invention also provides, the use of a compound of formula (I) or a pharmaceutically acceptable salt thereof to prepare a medicament for the treatment of a disease or condition, wherein the disease or condition is selected from the group consisting of: systemic diseases with an obvious inflammatory basis, inflammatory bowel disease, type 1 diabetes, Graves disease, multiple sclerosis, various types of arthritis, vasculitis, dermatitis, glomerulonephritis, hepatitis, periodonititis; and non-obvious inflammatory systemic disease with an inflammatory basis including, diabetes, atherosclerosis, heart failure, obesity, Alzheimer's disease, and metabolic syndrome.

The invention also provides, the use of a compound of formula (I) or a pharmaceutically acceptable salt thereof to prepare a medicament for the treatment of a disease or condition, wherein the disease or condition is selected from the group consisting of: ocular disease with an obvious inflammatory basis, including keratitis, endophthalmitis, blepharitis, conjunctivitis, scleritis, herpetic inflammation, uveitis, vasculitis, arteritis, orbital inflammations, optic neuritis, sympathetic ophthalmia, retinitis, and other autoimmune diseases; and non-obvious inflammatory ocular diseases with an inflammatory basis including, age-related macular degeneration, macular edema, diabetic retinopathy, glaucoma, proliferative vitreoretinopathy, and corneal, uveal, or retinal edema.

The invention also provides, the use of a compound of formula (I) or a pharmaceutically acceptable salt thereof to prepare a medicament for the treatment of a disease or condition, wherein the disease or condition is selected from the group consisting of: conditions and/or disorders characterized by angiogenesis, including ocular disease with an obvious angiogenesis basis, including diabetic retinopathy, age-related macular degeneration, retinopathy of prematurity, retinal artery or vein occlusion, corneal graft rejection, corneal neovascularization, neovascular glaucoma and sickle cell retinopathy; and non-ocular disease with an obvious angiogenesis basis, including, cancer, skin diseases, diabetic ulcers, diabetic nephropathy, cardiovascular disease and stroke.

The invention also provides, a compound or salt that modulates the activity of PPARα and STING.

The invention also provides, a compound or salt that has dual activity as an agonist of PPAR-alpha and as an inhibitor of STING.

The invention also provides, a compound selected from:

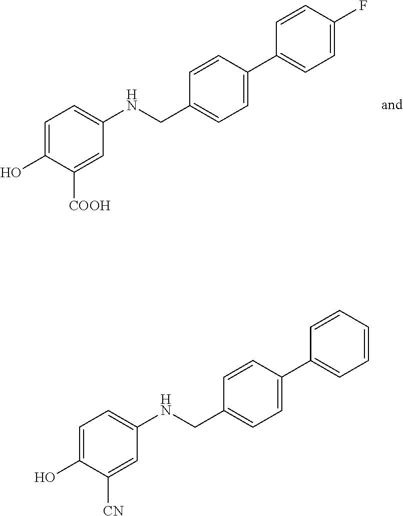 [SMILES: O=C(O)c1cc(NCc2ccc(-c3ccc(F)cc3)cc2)ccc1O ; N#Cc1cc(NCc2ccc(-c3ccccc3)cc2)ccc1O]

or a salt thereof.

The invention also provides, a method for treating a disease in an animal where both PPARα and STING are implicated and modulation of both targets may be beneficial, comprising administering to the animal, a compound or salt that is a dual modulator of PPARα and STING.

The invention also provides, a method for treating a disease in an animal where agonism of PPAR-alpha and inhibition of STING are both implicated, comprising administering to the animal, a compound or pharmaceutically acceptable salt that is an agonist of PPARα and an inhibitor of STING.

The invention also provides, a compound or salt that modulates the activity of PPARα and STING for use in medical therapy.

The invention also provides, a compound or salt that modulates the activity of PPARα and STING for the prophylactic or therapeutic treatment of a disease or condition where both PPARα and STING are implicated and modulation of both PPARα and STING may be beneficial.

The invention also provides, a compound or salt that has dual activity as an agonist of PPAR-alpha and as an inhibitor of STING for the prophylactic or therapeutic treatment of a disease or condition wherein agonism of PPAR-alpha and as an inhibition of STING may be beneficial.

The invention also provides, the use of a compound or salt that modulates the activity of PPARα and STING to prepare a medicament for treating a disease or condition where both PPARα and STING are implicated and modulation of both PPARα and STING may be beneficial.

The invention also provides, the use of a compound or salt that has dual activity as an agonist of PPAR-alpha and as an inhibitor of STING to prepare a medicament for treating a disease or condition wherein agonism of PPAR-alpha and as an inhibition of STING may be beneficial.

The invention also provides processes and intermediates disclosed herein that are useful for preparing a compound of formula (I) or a salt thereof.

### BRIEF DESCRIPTION OF THE FIGURES

FIGS. 1A-1B. Illustrates cell protection from palmitate challenge. (A) 661 Wcells or (B) MIO-M1 cells were treated with 25 μM of Example 17 (A229) or FA for 4 h, then 150 μM palmitate (PA) was added and co-incubated with the cells for another 24 h. Cell viability was determined by counting viable cells and statistical analysis (Example 40).

FIG. 2. Illustrates reduction of sVEGF production after CoCl₂ challenge. Example 17 (A229, 25 μM) treatment reduced CoCl₂-induced (200 μM) soluble VEGF (sVEGF) in ARPE19 cells (Example 40).

FIGS. 3A-3C. Illustrates protection against palmitate-mediated ROS production. Example 17 (A229, 25 μM) treatment decreased the ROS production under palmitate-induced (150 μM) retinal oxidative stress in (A) MIO-M1 cells, (B) AREP19 cells, and (C) hRPE cells (Example 40).

FIGS. 4A-4B. A) Example 31 (BH400)) reduces CoCl₂-induced production of ROS. HMC3 cells were pretreated with Example 31 and incubated with CoCl₂, then ROS production was measured by DCF fluorescence and normalized to total protein concentration (n=6). B) Example 31 reduces LPS-induced secretion of interleukin 6 (IL-6). HMC3 cells were pretreated with different concentrations of Example 31, then stimulated with LPS. The media was collected for ELISA and normalized by total protein concentration (n=3). Values are mean±SEM. *P<0.05, **P<0.01, ***P<0.001, ****P<0.0001, one-way ANOVA. (See Example 41)

FIGS. 5A-5E. A) Reduction in TBHP-induced production of ROS. HMC3 cells are treated with 12.5 μM test compounds and 200 μM TBHP. ROS production was measured by DCF fluorescence. B) Reduction in 4-HNE-induced production of ROS in 661 W cells. 661 W cells were pretreated with SN-011, Example 17 (A229), and Example 31 (BH400, 12.5 μM) for 2 hours, and 4-HNE (10 μM) for another 24 hours. ROS production was measured using H2DCFDA and normalized by total protein concentration (n=6). C) Reduction in H₂O₂-induced ROS production in 661 W cells. 661 W cells were pretreated with SN-011, Example 17 and Example 31 (12.5 μM) for 2 hours, and then exposed to H₂O₂ (500 μM) for another 24 hours. ROS production was measured using H2DCFDA and normalized by total protein concentration (n=6). D) Improvement in cell viability in 661 W cells measured by trypan blue exclusion after administration of 4-HNE. 661 W cells were treated with compounds SN-011 and Example 31 at a concentration of 1 μM and 10 μM for 2 hr, and then exposed to 4-HNE (10 μM) for another 24 hr. Cell viability was counted with Trypan Blue exclusion (n=3). E) Improvement in cell viability measured by trypan blue exclusion after exposure to H₂O₂. 661 W cells were treated with compounds SN-011 and Example 31 at a concentration of 1 μM and 10 μM for 2 hours, and then stressed with H₂O₂ (500 μM) for another 24 hours. Cell viability was counted with Trypan Blue exclusion (n=3). Values are mean±SEM. *P<0.05, **P<0.01, ***P<0.001, ****P<0.0001, one-way ANOVA or t-test. (See Example 42)

FIG. 6. Shows single-cell RNA sequencing showing PPARα, STING, and comparator expression in rod photoreceptors and endothelial cells. Rod photoreceptors show high gene expression of PPARA (144.6 nTPM), but endothelial cells show much lower expression (14.3 nTPM). Comparatively, STING1 expression is high in endothelial cells (99.6 nTPM) but nearly absent in rod photoreceptors (0.3 nTPM). NFKB1 is also shown for reference as a proinflammatory comparator protein.

### DETAILED DESCRIPTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C-s means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, isopropyl, n-butyl, t-butyl, iso-butyl, sec-butyl, n-pentyl, n-hexyl, n-heptyl, n-octyl, and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 6 carbon atoms.

The term “heteroaryl” as used herein refers to a single aromatic ring that has at least one atom other than carbon in the ring, wherein the atom is selected from the group consisting of oxygen, nitrogen and sulfur. The point of attachment for a heteroaryl ring can be at any suitable atom of the heteroaryl. Exemplary heteroaryls include but are not limited to pyridyl, pyrrolyl, pyrazinyl, pyrimidinyl, pyridazinyl, pyrazolyl, thienyl, imidazolyl, triazolyl, tetrazolyl, oxazolyl, isoxazolyl, thiazolyl, furyl, oxadiazolyl, and thiadiazolyl,

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” as used herein includes mammals (e.g., humans), fish, birds, reptiles, and amphibians.

The term “mammal” as used herein refers to humans, higher non-human primates, rodents, domestic, cows, horses, pigs, sheep, dogs and cats. In one embodiment, the mammal is a human.

The term “patient” as used herein refers to any animal including mammals. In one embodiment, the patient is a mammalian patient. In one embodiment, the patient is a human patient.

The term “a disease in an animal where both PPARα and STING are implicated and modulation of both targets may be beneficial “includes neurodegeneration, inflammation, vascular leakage, neovascularization, and fibrosis. The term also includes diabetic retinopathy and age-related macular degeneration.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine,) followed by the removal of 1 or more atoms from the second functional group to create the open valence.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₄)alkyl can be methyl, ethyl, propyl, isopropyl, butyl, iso-butyl, or sec-butyl; (C₃-C₆)cycloalkyl can be cyclopropyl, cyclobutyl, cyclopentyl, or cyclohexyl; and (C₁-C₄)alkoxy can be methoxy, ethoxy, propoxy, isopropoxy, butoxy, iso-butoxy, or sec-butoxy.

A specific compound or salt is a compound of formula (Ia):

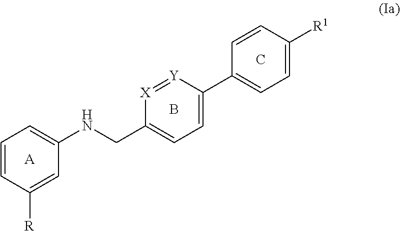

or a salt thereof, wherein:
R is —COOR², —CN, or 5-tetrazolyl;
X and Y are both CH; or one of X and Y is CH and the other is N.
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ib):

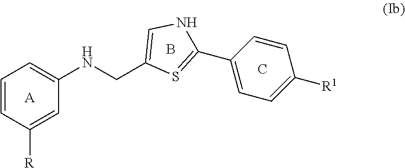

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{c}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ic):

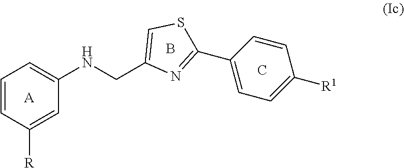

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, N^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₁-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Id):

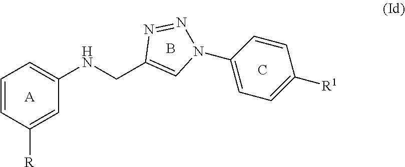

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ie):

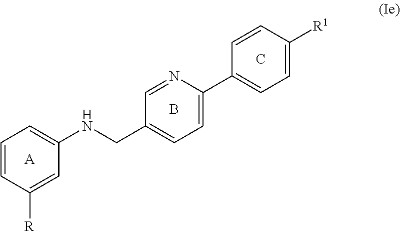

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b},
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, N^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₁-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (If′):

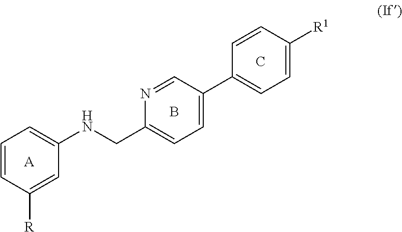

or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{x} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific value for R is —COOR².

A specific value for R is —CN.

A specific value for R is 5-tetrazolyl.

A specific compound or salt is a compound of formula (Ia′):

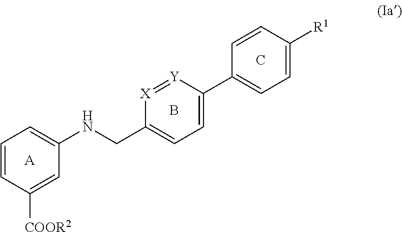

or a salt thereof, wherein:
X and Y are both CH; or one of X and Y is CH and the other is N.
ring A is additionally optionally substituted with one or more groups independently selected from the group consisting of halo, hydroxy, (C₁-C₄)alkyl, (C₁-C₆)alkoxy, and NR^{c}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ib′):

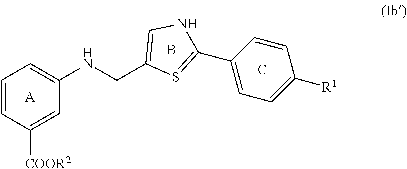

or a salt thereof, wherein
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁₋₄)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ic′):

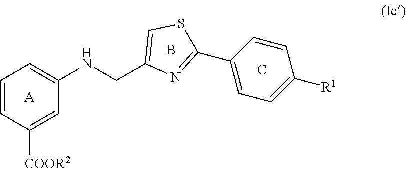

or a salt thereof, wherein
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₁-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Id′):

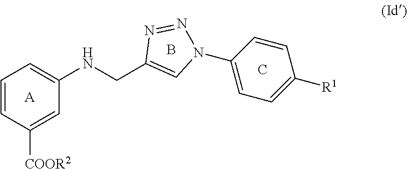

or a salt thereof, wherein
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or Rand R^{d} together with the nitrogen to which they are attached form a aziridino, azetidino, morpholino, piperazino, pyrrolidino or piperidino.

A specific compound or salt is a compound of formula (Ie′):

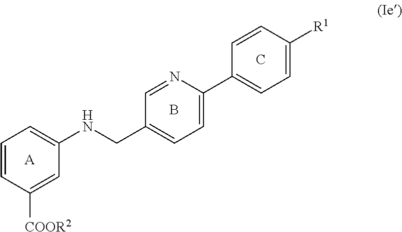

or a salt thereof, wherein
ring A is additionally optionally substituted with one or more groups independently selected from the group consisting of halo, hydroxy, (C₁-C₄)alkyl, (C₁-C₆)alkoxy, and NR^{a}R^{b},
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{x} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (If″):

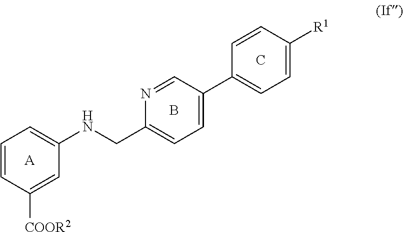

or a salt thereof, wherein
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific compound or salt is a compound of formula (Ig):

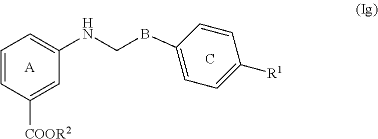

or a salt thereof, wherein:
ring A is additionally optionally substituted with one or more groups independently selected from the group consisting of halo, hydroxy, (C₁-C₄)alkyl, (C₁-C₆)alkoxy, and NR^{a}R^{b};
B is phenyl, a 5-membered heteroaryl, or a 6-membered heteroaryl;
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
B is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

A specific value for B is phenyl that is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

A specific value for B is a 5-membered heteroaryl that is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

A specific value for B is a 6-membered heteroaryl that is additionally optionally substituted with one or more groups R that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

A specific value for ring A is additionally optionally substituted with one or more groups independently selected from the group consisting of halo, hydroxy, and (C₁-C₆)alkoxy.

A specific value for ring A is additionally optionally substituted with one or more groups independently selected from the group consisting of halo.

A specific value for ring A is:

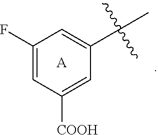

A specific value for ring A is additionally optionally substituted with hydroxy.

A specific value for ring A is:

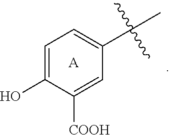

A specific value for X and Y are both CH.

A specific value for X is CH and Y is N.

A specific value for X is N and Y is CH.

A specific value for ring B is substituted with one or more groups R^{x} that are independently selected from the group consisting methoxy, methyl, hydroxy, and chloro.

A specific value for R¹ is fluoro, chloro, methyl, trifluoromethyl, methoxy, trifluoromethoxy, cyano, or cyclopentyl.

A specific value for ring C is not optionally substituted with one or more groups R^{y}.

A specific value for R² is H.

A specific value for R² is (C₁-C₄)alkyl.

A specific value for R² is methyl.

Processes for preparing compounds of formula (I) are provided as further embodiments of the invention.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of diabetic retinopathy. Examples of such agents include anti-VEGF agents and corticosteroids. Compounds of the invention can also be administered in combination with other therapeutic agents that are useful for the treatment of liver diseases or pain. Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising a compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to an animal to treat diabetic retinopathy.

### Heterobifunctional PPARα/STING Modulators

In one embodiment, dual modulators of PPARα and STING (Stimulator of Interferon Genes) are provided. Such dual modulators may be particularly useful for treating diseases where both targets are implicated, and modulation of both targets may be beneficial.

The compound of Example 31 exhibits heterobifunctional activity in cell-based assays, agonizing PPARα (EC₅₀=1.2μ) and inhibiting STING (IC₅₀=8.1 μM). This compound demonstrates superior protection against oxidative stress compared to single-target PPARα or STING modulation in the HMC3 microglial cell line and improved cell viability following treatment with chemical oxidants in the 661 W photoreceptor cell line.

In one embodiment, a compound or salt that is a dual modulator of PPARα and STING is provided. In one embodiment, a compound or salt with dual activity as an agonist of PPAR-alpha and as an inhibitor of STING is provided. In one embodiment, a method for treating a disease or condition in an animal where both PPARα and STING are implicated and modulation of both targets may be beneficial, comprising administering to the animal a compound or salt that is a dual modulator of PPARα and STING is provided. In one embodiment, a method for treating a disease or condition in an animal where agonism of PPAR-alpha and inhibition of STING are both implicated and agonism of PPAR-alpha and inhibition of STING may be beneficial, comprising administering to the animal, a compound or pharmaceutically acceptable salt that is a dual modulator of PPARα and STING is provided. In one embodiment, the compound or salt is a compound of formula (I) or a pharmaceutically acceptable salt thereof, wherein Ring A has the following structure:

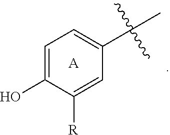

In one embodiment, the compound or salt is selected from:

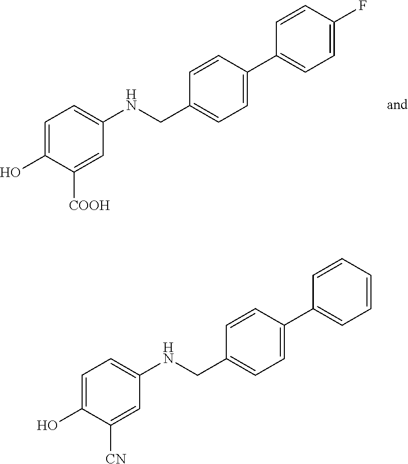 [SMILES: O=C(O)c1cc(NCc2ccc(-c3ccc(F)cc3)cc2)ccc1O ; N#Cc1cc(NCc2ccc(-c3ccccc3)cc2)ccc1O]

and pharmaceutically acceptable salts thereof. In one embodiment, the disease where agonism of PPAR-alpha and inhibition of STING are both implicated and agonism of PPAR-alpha and inhibition of STING may be beneficial is an inflammatory retinal disease, pain, or a liver disease (e.g., fibrosis, NASH, NAFLD or MASLD).

In one embodiment, the disease where agonism of PPAR-alpha and inhibition of STING are both implicated and agonism of PPAR-alpha and inhibition of STING may be beneficial is diabetic retinopathy (DR) or age-related macular degeneration (AMD).

The invention will now be illustrated by the following non-limiting Examples.

### EXAMPLES

### Examples 1-7

Scheme 1. General Synthetic Approach for Analogues 3b-3h

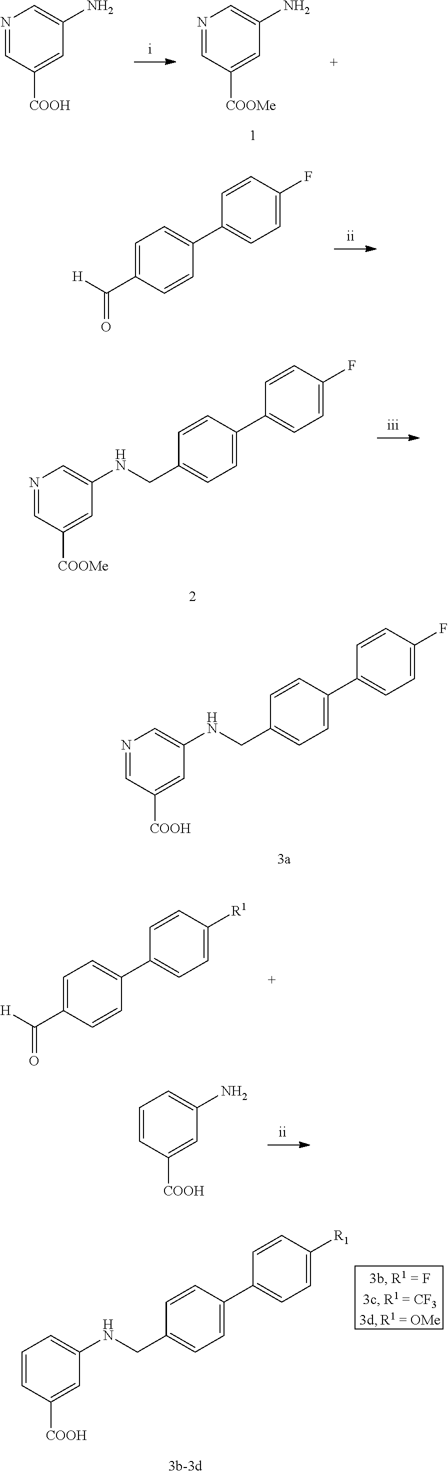

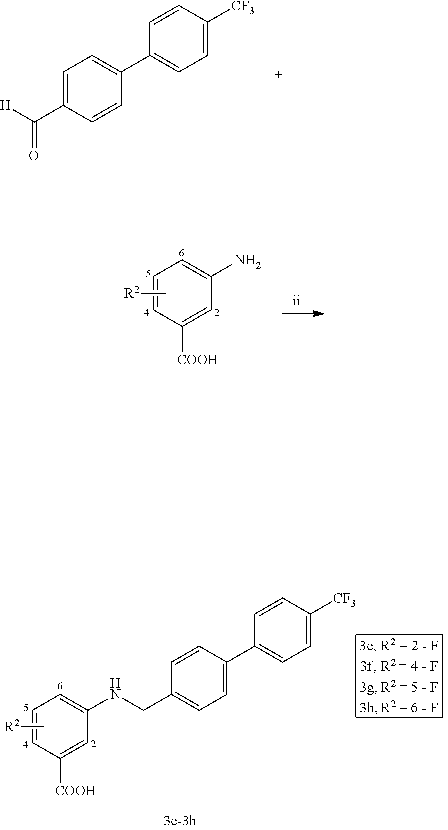

(i) SOCl₂, MeOH, 0° C. to reflux, 16 h; (ii) (a) toluene, 115° C., 1-2 h; (b) HOAc, NaBH(OAc)₃, THF, 0-25° C. 14-16 h; (iii) LiOH·H₂O, THF/MeOH/H₂O (v/v/v=3:1:1), 25° C., 2-16 h.

### Example 1. Preparation of 3-(((4′-Fluoro-[1,1′-biphenyl]-4-yl)methyl)amino)benzoic acid (3b)

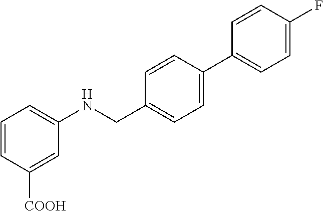 [SMILES: O=C(O)c1cccc(NCc2ccc(-c3ccc(F)cc3)cc2)c1]

A mixture of 4′-fluoro-[1,1′-biphenyl]-4-carbaldehyde (37 mg, 0.19 mmol) and 3-aminobenzoic acid (25 mg, 0.18 mmol) in toluene (2 mL) was stirred at 115° C. for 2 hours. The solvent was removed under reduced pressure by rotary evaporation. The residue was dissolved in THF (2 mL) and cooled to 0° C. Sodium triacetoxyborohydride (77 mg, 0.36 mmol) was added and the mixture was stirred at 0° C. for 15 minutes. Acetic acid (1 μL) was added and the mixture was then warmed to room temperature and stirred. After the reaction was judged to be completed by TLC (16 h), it was quenched with water and extracted with EtOAc. The organic layer was washed with water and brine and concentrated under reduced pressure by rotary evaporation. The crude residue was purified by flash column chromatography (SiO₂: 0-20% EtOAc/Hexane with 1% AcOH). The appropriate fractions were collected and concentrated under reduced pressure to afford compound 3b (47 mg, 80%). ¹H NMR (400 MHz, Acetone-d₆) δ 7.71-7.65 (m, 2H), 7.63-7.57 (m, 2H), 7.50 (d, J=8.0 Hz, 2H), 7.41-7.36 (m, 1H), 7.29 (dt, J=7.7, 1.3 Hz, 1H), 7.21 (ddd, J=8.9, 6.8, 2.4 Hz, 3H), 6.91 (ddd, J=8.1, 2.6, 1.1 Hz, 1H), 4.47 (s, 2H). ¹³C NMR (101 MHz, Acetone-d₆) δ 168.3, 163.4 (d, J=244.3 Hz), 149.9, 140.2, 139.5, 138.1 (d, J=3.1 Hz), 132.4, 129.9, 129.6 (d, J=8.1 Hz, 2C), 128.8 (2C), 127.8 (2C), 118.7, 117.8, 116.5 (d, J=21.6 Hz, 2C), 114.5, 47.7. MSESI m/z: 320.1099 (C₂₀H₁₅FNO₂⁻ requires 320.1092).

### Example 2. Preparation of 3-(((4′-(Trifluoromethyl)-[1,1′-biphenyl]-4-yl)methyl)amino)benzoic acid (3c)

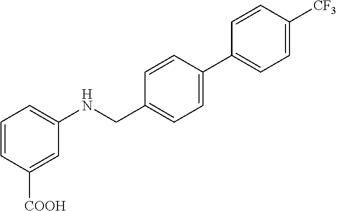 [SMILES: O=C(O)c1cccc(NCc2ccc(-c3ccc(C(F)(F)F)cc3)cc2)c1]

A mixture of 4′-(trifluoromethyl)-[1,1′-biphenyl]-4-carbaldehyde (47 mg, 0.19 mmol) and 3-aminobenzoic acid (25 mg, 0.18 mmol) in toluene (2 mL) was stirred at 115° C. for 2 hours. The solvent was removed under reduced pressure by rotary evaporation. The residue was dissolved in THF (2 mL) and cooled to 0° C. Sodium triacetoxyborohydride (77 mg, 0.36 mmol) was added and the mixture was stirred at 0° C. for 15 minutes. Acetic acid (1 μL) was added and the mixture was then warmed to room temperature and stirred. After the reaction was judged to be completed by TLC (16 h), it was quenched with water and extracted with EtOAc. The organic layer was washed with water and brine and concentrated under reduced pressure by rotary evaporation. The crude residue was purified by flash column chromatography (SiO₂: 0-20% EtOAc/Hexane with 1% AcOH). The appropriate fractions were collected and concentrated under reduced pressure to afford compound 3c (66 mg, 97%). ¹H NMR (400 MHz, Acetone-d₆) δ 7.88 (d, J=8.2 Hz, 2H), 7.79 (d, J=8.2 Hz, 2H), 7.73-7.68 (m, 2H), 7.59-7.53 (m, 2H), 7.39 (dd, J=2.6, 1.5 Hz, 1H), 7.30 (dt, J=7.6, 1.3 Hz, 1H), 7.21 (t, J=7.8 Hz, 1H), 6.92 (ddd, J=8.1, 2.6, 1.1 Hz, 1H), 5.89 (s, 1H), 4.50 (s, 2H). ¹³C NMR (101 MHz, Acetone-d₆) δ 168.2, 149.7, 145.5, 141.4, 138.8, 132.3, 129.8, 129.5 (q, J=32.1 Hz), 128.9 (2C), 128.3 (2C), 128.1 (2C), 126.6 (q, J=3.9 Hz, 2C), 125.5 (q, J=270.9 Hz), 118.7, 117.7, 114.3, 47.5. MSESI m/z: 370.1052 (C₂₁H₁₅F₃NO₂⁻ requires 370.1055).

### Example 3. Preparation of 3-(((4′-Methoxy-[1,1′-biphenyl]-4-yl)methyl)amino)benzoic acid (3d)

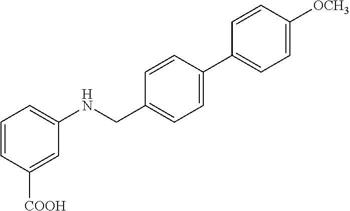 [SMILES: COc1ccc(-c2ccc(CNc3cccc(C(=O)O)c3)cc2)cc1]

A mixture of 4′-methoxy-[1,1′-biphenyl]-4-carbaldehyde (18 mg, 85 μmol) and 3-aminobenzoic acid (12 mg, 85 μmol) in toluene (2 mL) was stirred at 115° C. for 1 hour. The solvent was removed under reduced pressure by rotary evaporation. The residue was dissolved in THF (2 mL) and cooled to 0° C. Sodium triacetoxyborohydride (36 mg, 0.17 mmol) was added and the mixture was stirred at 0° C. for 15 minutes. Acetic acid (1 μL) was added and the mixture was then warmed to room temperature and stirred. After the reaction was judged to be completed by TLC (16 h), it was quenched with water and extracted with EtOAc. The organic layer was washed with water and brine and concentrated under reduced pressure by rotary evaporation. The crude residue was purified by flash column chromatography (SiO₂: 0-20% EtOAc/Hexane with 1% AcOH). The appropriate fractions were collected and concentrated under reduced pressure to afford compound 3d (22 mg, 78%). ¹H NMR (400 MHz, DMSO-d₆) δ 7.61-7.54 (m, 4H), 7.40 (d, J=8.3 Hz, 2H), 7.21-7.18 (m, 1H), 7.16-7.08 (m, 2H), 7.04-6.96 (m, 2H), 6.79 (dt, J=7.8, 1.9 Hz, 1H), 6.54 (t, J=6.1 Hz, 1H), 4.32 (d, J=5.8 Hz, 2H), 3.78 (s, 3H). ¹³C NMR (101 MHz, DMSO-d₆) δ 168.0, 158.8, 148.7, 138.4, 138.3, 132.4, 131.9, 128.8, 127.7 (2C), 127.6 (2C), 126.1 (2C), 116.8, 116.1, 114.3 (2C), 112.9, 55.1, 46.0. MSESI m/z: 332.1286 (C₂₁H₁₈NO₃⁻ requires 332.1287).

### General Procedure 1

A mixture of 4′-(trifluoromethyl)-[1,1′-biphenyl]-4-carbaldehyde (41 mg, 0.16 mmol) and amino fluorobenzoic acid (25 mg, 0.16 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxyborohydride (68 mg, 0.32 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-20% EtOAc/Hexane with 1% AcOH).

### Example 4. Preparation of 2-Fluoro-3-(((4′-(trifluoromethyl)-[1,1′-biphenyl]-4-yl)methyl)amino)benzoic acid (3e)

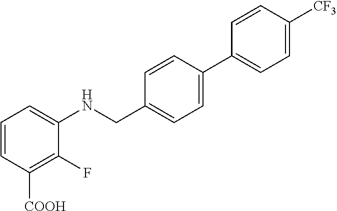 [SMILES: O=C(O)c1cccc(NCc2ccc(-c3ccc(C(F)(F)F)cc3)cc2)c1F]

Using general procedure 1, employing 4′-(trifluoromethyl)-[1,1′-biphenyl]-4-carbaldehyde (41 mg, 0.16 mmol) and 3-amino-2-fluorobenzoic acid. (25 mg, 0.16 mmol) Compound 3e was obtained after purification. (30 mg, 48%). ¹H NMR (400 MHz, DMSO-d₆) δ 13.01 (s, 1H), 7.87 (d, J=8.2 Hz, 2H), 7.79 (d, J=8.3 Hz, 2H), 7.73-7.65 (m, 2H), 7.49 (d, J=8.1 Hz, 2H), 6.97-6.89 (m, 2H), 6.76 (ddd, J=7.9, 6.2, 3.5 Hz, 1H), 6.52 (td, J=6.3, 2.6 Hz, 1H), 4.43 (d, J=6.2 Hz, 2H). ¹³C NMR (101 MHz, DMSO-d₆) δ 165.7, 149.7 (d, J=251.6 Hz), 144.0, 140.2, 137.3 (d, J=12.1 Hz), 137.0, 127.7 (2C), 127.6 (q, J=31.5 Hz), 127.3 (2C), 127.1 (2C), 125.7 (q, J=3.7 Hz, 2C), 124.0 (d, J=4.0 Hz), 123.0, 118.9 (d, J=8.4 Hz), 116.8, 115.5 (d, J=4.4 Hz), 45.4. MSESI m/z: 388.0959 (C₂₁H₁₄F₄NO₂⁻ requires 388.0961).

### Example 5. Preparation of 2-Fluoro-5-(((4′-(trifluoromethyl)-[1,1′-biphenyl]-4-yl)methyl)amino)benzoic acid (3f)

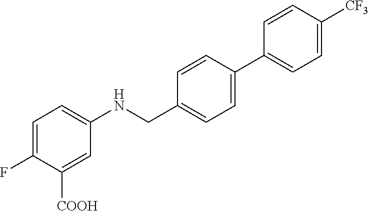 [SMILES: O=C(O)c1cc(NCc2ccc(-c3ccc(C(F)(F)F)cc3)cc2)ccc1F]

Using general procedure 1, employing 4′-(trifluoromethyl)-[1,1′-biphenyl]-4-carbaldehyde (41 mg, 0.16 mmol) and 5-amino-2-fluorobenzoic acid (25 mg, 0.16 mmol). Compound 3f was obtained after purification. (60 mg, 96%). ¹H NMR (400 MHz, DMSO-d₆) δ 12.99 (s, 1H), 7.89 (d, J=8.4 Hz, 2H), 7.80 (d, J=8.7 Hz, 2H), 7.75-7.68 (m, 2H), 7.52-7.46 (m, 2H), 7.07-7.02 (m, 1H), 7.02-6.97 (m, 1H), 6.79 (dt, J=9.0, 3.7 Hz, 1H), 6.50 (t, 1H), 4.34 (d, J=5.6 Hz, 2H). ¹³C NMR (101 MHz, DMSO-d₆) δ 165.6 (d, J=2.9 Hz), 153.0 (d, J=244.7 Hz), 144.9 (d, J=2.2 Hz), 143.9, 140.3, 137.0, 127.9 (2C), 127.7 (q, J=31.9 Hz), 127.3 (2C), 127.1 (2C), 125.7 (q, J=3.9 Hz, 2C), 123.0, 119.2 (d, J=11.4 Hz), 117.4 (d, J=7.7 Hz), 117.1 (d, J=23.5 Hz), 113.7, 46.3. MSESI m/z: 388.0959 (C₂₁H₁₄F₄NO₂⁻ requires 388.0961).

### Example 6. Preparation of 3-Fluoro-5-(((4′-(trifluoromethyl)-[1,1′-biphenyl]-4-yl)methyl)amino)benzoic acid (3g)

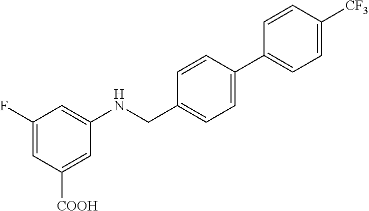 [SMILES: O=C(O)c1cc(F)cc(NCc2ccc(-c3ccc(C(F)(F)F)cc3)cc2)c1]

Using general procedure 1, employing 4′-(trifluoromethyl)-[1,1′-biphenyl]-4-carbaldehyde (41 mg, 0.16 mmol) and 3-amino-5-fluorobenzoic acid (25 mg, 0.16 mmol). Compound 3g was obtained after purification. (60 mg, 96%). ¹H NMR (400 MHz, Acetone-d₆) δ 8.33 (d, J=8.2 Hz, 2H), 8.24 (d, J=8.2 Hz, 2H), 8.17 (d, J=8.3 Hz, 1H), 8.01 (d, J=8.2 Hz, 2H), 7.73-7.65 (m, 1H), 7.37 (ddd, J=9.2, 2.4, 1.3 Hz, 1H), 7.08 (dt, J=11.5, 2.3 Hz, 1H), 6.69 (s, 1H), 4.96 (s, 2H). ¹³C NMR (101 MHz, Acetone-d₆) δ 167.1, 164.6 (d, J=240.9 Hz), 151.6 (d, J=11.1 Hz), 145.5 (d, J=1.6 Hz), 140.7, 139.0, 134.0 (d, J=9.7 Hz), 129.5 (q, J=32.1 Hz), 128.9 (2C), 128.3 (2C), 128.2 (2C), 126.6 (q, J=3.9 Hz, 2C), 125.5 (q, J=271.1 Hz), 111.1, 104.3 (d, J=23.9 Hz), 103.5 (d, J=25.8 Hz), 47.5. MSESI m/z: 388.0960 (C₂₁H₁₄F₄NO₂⁻ requires 388.0961).

### Example 7. Preparation of 4-Fluoro-3-(((4′-(trifluoromethyl)-[1,1′-biphenyl]-4-yl)methyl)amino)benzoic acid (3h)

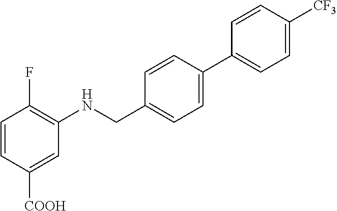 [SMILES: O=C(O)c1ccc(F)c(NCc2ccc(-c3ccc(C(F)(F)F)cc3)cc2)c1]

Using general procedure 1, employing 4′-(trifluoromethyl)-[1,1′-biphenyl]-4-carbaldehyde (41 mg, 0.16 mmol) and 3-amino-4-fluorobenzoic acid (25 mg, 0.16 mmol). Compound 3 h was obtained after purification. (30 mg, 48%). ¹H NMR (400 MHz, Acetone-d₆) δ 7.88 (d, J=8.2 Hz, 2H), 7.78 (d, J=8.3 Hz, 2H), 7.74-7.69 (m, 2H), 7.58 (d, J=8.1 Hz, 2H), 7.38 (dd, J=8.6, 2.1 Hz, 1H), 7.33 (ddd, J=8.3, 4.8, 2.0 Hz, 1H), 7.11 (dd, J=11.6, 8.3 Hz, 1H), 5.84 (s, 1H), 4.60 (d, J=5.3 Hz, 2H). ¹³C NMR (101 MHz, Acetone-d₆) δ 167.4, 155.1 (d, J=245.0 Hz), 145.5, 140.9, 138.9, 137.6 (d, J=12.1 Hz), 129.5 (q, J=32.3 Hz), 128.7 (2C), 128.3 (2C), 128.2 (2C), 126.6 (q, J=4.0 Hz, 2C), 125.5 (q, J=271.1 Hz), 119.4 (d, J=8.1 Hz), 115.1 (d, J=19.8 Hz), 114.3 (d, J=5.1 Hz), 47.1. MSESI m/z: 388.0958 (C₂₁H₁₄F₄NO₂⁻ requires 388.0961).

### Examples 8-21

General Procedure 2: Synthesis of Aldehyde Intermediates 4a-4k, 4m-4o.

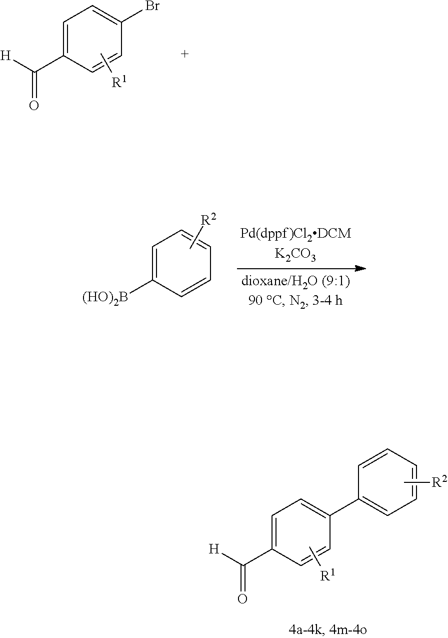

A mixture of substituted 4-bromobenzaldehyde (0.2 mmol), aryl boronic acid (0.24 mmol), potassium carbonate (110 mg, 0.8 mmol), and Pd(dppf)Cl₁·CH₂Cl₂ (16 mg, 0.02 mmol) in dioxane/H₂O (v/v=9:1, 1 mL) was stirred at 90° C. under a nitrogen atmosphere for 3-4 hours. The reaction was cooled to room temperature, quenched with water, and extracted with EtOAc. The organic layer was washed with water and brine and concentrated under reduced pressure by rotary evaporation. The crude residue was purified by flash column chromatography (SiO₂) using an appropriate.

General Synthetic Approach for Compounds 6a-6n

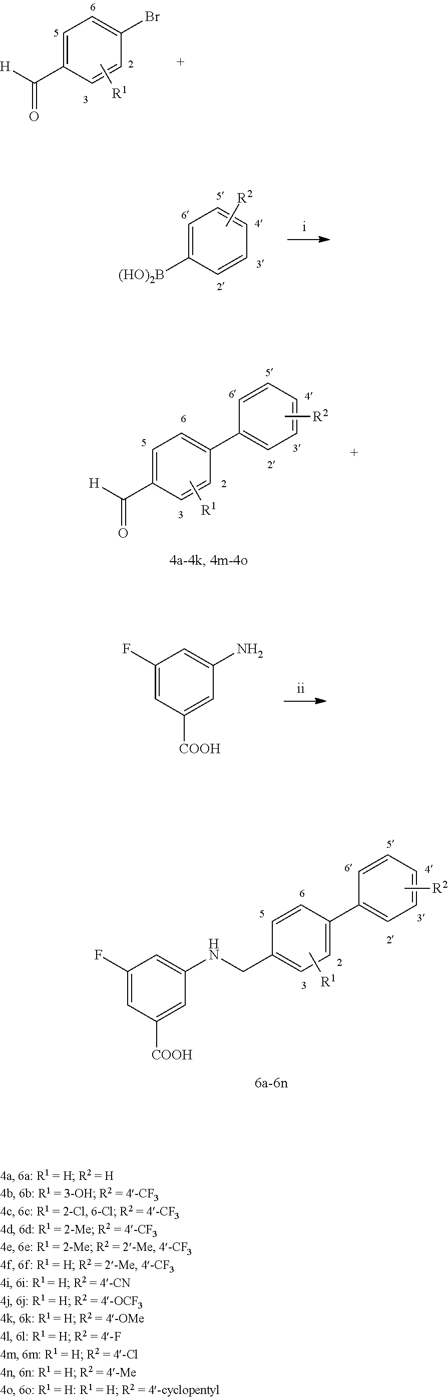

(i) Pd(dppf)₂Cl₂·DCM, K₂CO₃, dioxane/H₂O (v/v=9:1), N₂, 90° C., 3-4 h;
(ii) (a) toluene, 115° C., 1-2 h; (b) HOAc, NaBH(OAc)₃, THF, 0-25° C., 14-16 h;

### Example 8. Preparation of 3-(([1,1′-Biphenyl]-4-ylmethyl)amino)-5-fluorobenzoic Acid (6a)

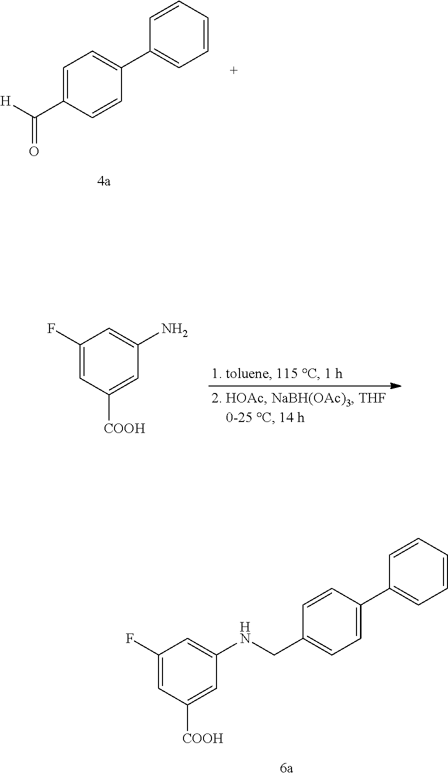 [SMILES: O=Cc1ccc(-c2ccccc2)cc1 ; Nc1cc(F)cc(C(=O)O)c1 ; O=C(O)c1cc(F)cc(NCc2ccc(-c3ccccc3)cc2)c1]

A mixture of 4a (27 mg, 0.15 mmol) and 3-amino-5-fluorobenzoic acid (23 mg, 0.15 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (63 mg, 0.3 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM) to afford compound 6a as a yellow amorphous solid (35 mg, 74%). ¹H NMR (400 MHz, DMSO-d₆) δ 12.99 (s, 1H), 7.70-7.58 (m, 4H), 7.55-7.41 (m, 4H), 7.39-7.28 (m, 1H), 7.13-7.02 (m, 1H), 6.94 (t, J=5.8 Hz, 1H), 6.77 (ddd, J=9.3, 2.3, 1.3 Hz, 1H), 6.57 (dt, J=11.8, 2.3 Hz, 1H), 4.35 (d, J=5.8 Hz, 2H). ¹³C NMR (101 MHz, DMSO-d₆) δ 166.8 (d, J=3.6 Hz), 163.1 (d, J=240.2 Hz), 150.6 (d, J=11.2 Hz), 139.9, 138.8, 138.6, 133.2 (d, J=9.7 Hz), 128.9, 127.8, 127.3, 126.7, 126.6, 109.9, 102.3 (d, J=23.5 Hz), 102.0 (d, J=25.6 Hz), 45.9. MSESI m/z: 320.1087 (C₂₀H₁₅FNO₂⁻ requires 320.1087).

The intermediate compound 4a was prepared as follows.

### a. Preparation of [1,1′-Biphenyl]-4-carbaldehyde (4a)

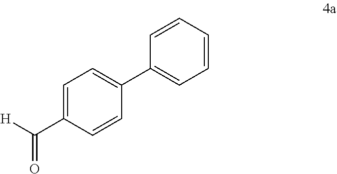 [SMILES: O=Cc1ccc(-c2ccccc2)cc1]

Using general procedure 2, employing 4-bromobenzaldehyde (37 mg, 0.2 mmol) and phenylboronic acid (29 mg, 0.24 mmol), 3 hours, compound 4a was obtained after flash column chromatography (SiO₂, 0-10% EtOAc in hexanes) as a yellow amorphous solid (31 mg, 85%). ¹H NMR (400 MHz, CDCl₃) δ 10.07 (s, 1H), 7.96 (d, J=8.4 Hz, 1H), 7.76 (d, J=8.4 Hz, 2H), 7.68-7.63 (m, 2H), 7.54-7.39 (m, 3H).

### Example 9. Preparation of 3-Fluoro-5-(((3-hydroxy-4′-(trifluoromethyl)-[1,1′-biphenyl]-4-yl)methyl)amino)benzoic Acid (6b)

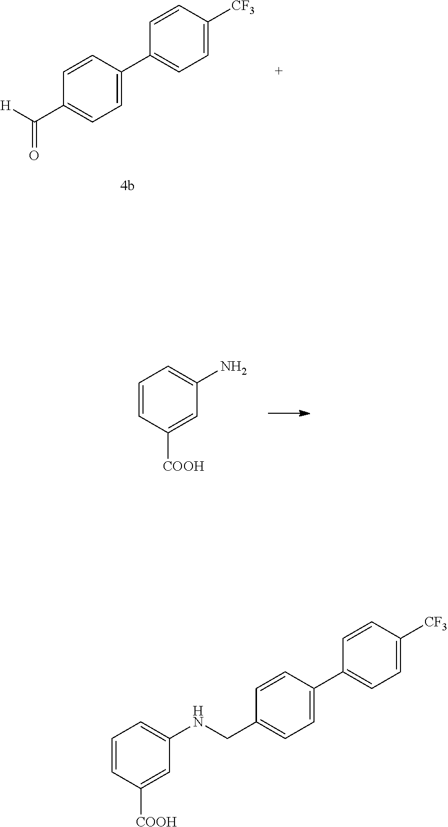 [SMILES: O=Cc1ccc(-c2ccc(C(F)(F)F)cc2)cc1 ; Nc1cccc(C(=O)O)c1 ; O=C(O)c1cccc(NCc2ccc(-c3ccc(C(F)(F)F)cc3)cc2)c1]

A mixture of 4b (29 mg, 0.11 mmol) and 3-amino-5-fluorobenzoic acid (17 mg, 0.11 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (46 mg, 0.22 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-10% MeOH in DCM) to afford compound 6b as a white amorphous solid (30 mg, 68%). ¹H NMR (400 MHz, DMSO-d₆) δ 10.02 (s, 1H), 8.02-7.69 (m, 4H), 7.29 (d, J=7.8 Hz, 1H), 7.16 (d, J=1.8 Hz, 1H), 7.12 (dd, J=7.8, 1.8 Hz, 1H), 7.08-7.05 (m, 1H), 6.77-6.70 (m, 2H), 6.55 (dt, J=11.8, 2.3 Hz, 1H), 4.27 (d, J=5.6 Hz, 2H). ¹³C NMR (101 MHz, DMSO-d₆) δ 166.9, 163.1 (d, J=239.9 Hz), 155.6, 150.7 (d, J=11.3 Hz), 144.2, 138.3, 133.7, 128.9, 127.7 (q, J=31.8 Hz), 127.2 (2C), 125.8 (q, J=3.7 Hz, 2C), 125.5, 124.4 (q, J=271.8 Hz), 117.7, 113.4, 109.6, 102.2 (d, J=23.5 Hz), 101.7 (d, J=25.5 Hz), 40.9. MSESI m/z: 404.0919 (C₂₁H₁₄F₄NO₃⁻ requires 404.0910).

The intermediate compound 4b was prepared as follows.

### a. Preparation of 3-Hydroxy-4′-(trifluoromethyl)-[1,1′-biphenyl]-4-carbaldehyde (4b)

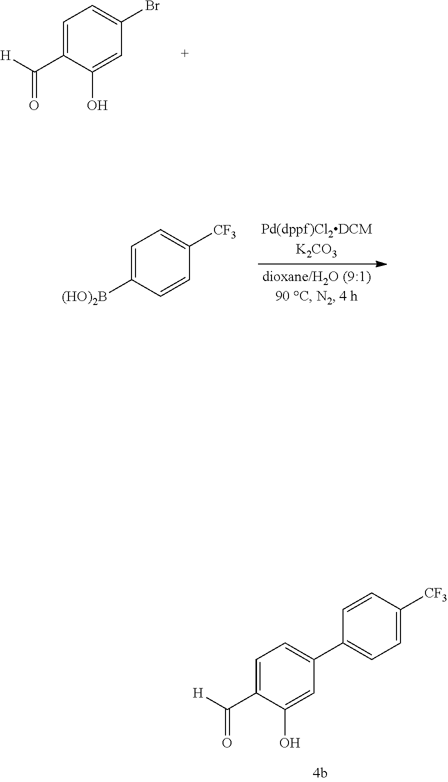 [SMILES: O=Cc1ccc(Br)cc1O ; OB(O)c1ccc(C(F)(F)F)cc1 ; O=Cc1ccc(-c2ccc(C(F)(F)F)cc2)cc1O]

A mixture of 4-bromo-2-hydroxybenzaldehyde (120 mg, 0.6 mmol), (4-(trifluoromethyl)-phenyl)boronic acid (136 mg, 0.72 mmol), potassium carbonate (330 mg, 2.4 mmol), and Pd(dppf)Cl₂·CH₂Cl₂ (49 mg, 0.06 mmol) in dioxane/H₂O (v/v=9:1, 3 mL) was stirred at 90° C. under a nitrogen atmosphere for 4 hours. The reaction was cooled to room temperature, quenched with water, and extracted with EtOAc. The organic layer was washed with water and brine and concentrated under reduced pressure by rotary evaporation. The crude residue was purified by flash column chromatography (SiO₂, 0-15% EtOAc/hexanes) to give compound 4b as a yellow amorphous solid (100 mg, 63%). ¹H NMR (400 MHz, CDCl₃) δ 11.12 (s, 1H), 9.95 (s, 1H), 7.72 (s, 4H), 7.66 (d, J=8.0 Hz, 1H), 7.25 (dd, J=8.0 Hz, J=1.6 Hz, 1H), 7.22 (d, J=1.6 Hz, 1H).

### Example 10. Preparation of 3-(((2,6-Dichloro-4′-(trifluoromethyl)-[1,1′-biphenyl]-4-yl)methyl)amino)-5-fluorobenzoic acid (6c)

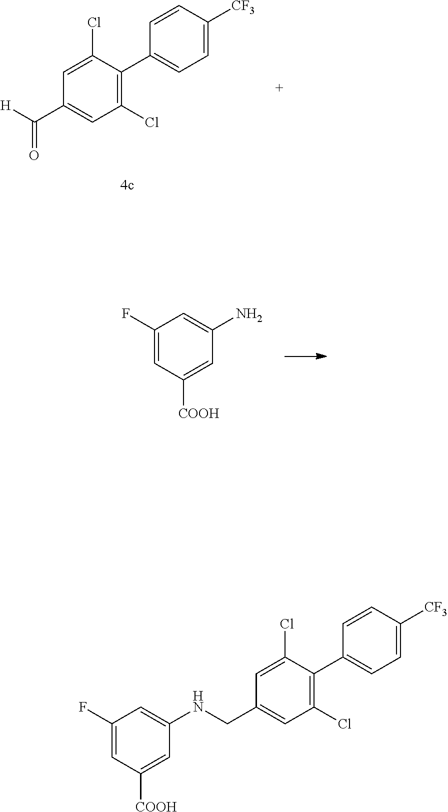 [SMILES: O=Cc1cc(Cl)c(-c2ccc(C(F)(F)F)cc2)c(Cl)c1 ; Nc1cc(F)cc(C(=O)O)c1 ; O=C(O)c1cc(F)cc(NCc2cc(Cl)c(-c3ccc(C(F)(F)F)cc3)c(Cl)c2)c1]

A mixture of 4c (43 mg, 0.13 mmol) and 3-amino-5-fluorobenzoic acid (21 mg, 0.13 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (57 mg, 0.27 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6c as a yellow amorphous solid (35 mg, 57%). ¹H NMR (400 MHz, DMSO-d₆) δ 7.85 (d, J=8.4 Hz, 2H), 7.60 (s, 2H), 7.53 (d, J=8.0 Hz, 2H), 7.10 (dd, J=2.1, 1.4 Hz, 1H), 7.01 (t, J=6.7 Hz, 1H), 6.81 (ddd, J=9.3, 2.4, 1.4 Hz, 1H), 6.63 (dt, J=11.7, 2.4 Hz, 1H), 4.41 (d, J=6.3 Hz, 2H). ¹³C NMR (101 MHz, DMSO-d₆) δ 166.7, 163.2 (d, J=240.6 Hz), 150.1 (d, J=11.0 Hz), 143.3, 140.4, 135.8, 133.4 (2C), 130.6 (2C), 128.8 (q, J=31.9 Hz), 127.0 (2C), 125.4 (q, J=3.7 Hz, 2C), 124.1 (q, J=272.2 Hz), 110.0, 102.8 (d, J=23.1 Hz), 102.1 (d, J=25.3 Hz), 44.8. MSESI m/z: 456.0181 (C₂₁H₁₂Cl₂F₄NO₂⁻ requires 456.0181).

The intermediate compound 4c was prepared as follows.

### a. Preparation of 2,6-Dichloro-4′-(trifluoromethyl)-[1,1′-biphenyl]-4-carbaldehyde (4c)

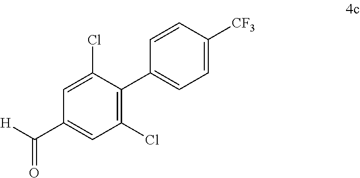 [SMILES: O=Cc1cc(Cl)c(-c2ccc(C(F)(F)F)cc2)c(Cl)c1]

Using general procedure 2, employing 4-bromo-3,5-dichlorobenzaldehyde (50 mg, 0.2 mmol) and (4-(trifluoromethyl)phenyl)boronic acid (45 mg, 0.24 mmol), 4 hours, compound 4c was obtained after flash column chromatography (SiO₂, 0-15% EtOAc in hexanes) as a yellow oil. (46 mg, 73%) ¹H NMR (400 MHz, CDCl₃) δ 9.99 (s, 1H), 7.93 (s, 2H), 7.77 (dd, J=8.7, 0.7 Hz, 2H), 7.40 (dd, J=8.8, 0.8 Hz, 2H).

### Example 11. Preparation of 3-Fluoro-5-(((2-methyl-4′-(trifluoromethyl)-[1,1′-biphenyl]-4-yl)methyl)amino)benzoic Acid (6d)

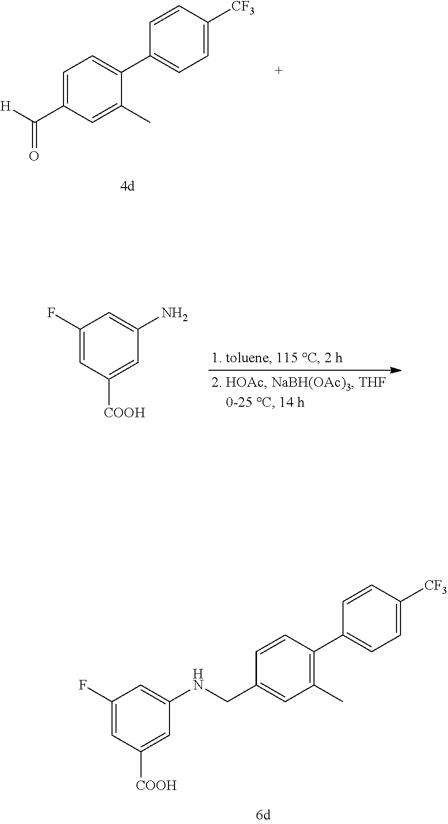 [SMILES: Cc1cc(C=O)ccc1-c1ccc(C(F)(F)F)cc1 ; Nc1cc(F)cc(C(=O)O)c1 ; Cc1cc(CNc2cc(F)cc(C(=O)O)c2)ccc1-c1ccc(C(F)(F)F)cc1]

A mixture of 4d (22 mg, 0.083 mmol) and 3-amino-5-fluorobenzoic acid (13 mg, 0.083 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (35 mg, 0.17 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) followed by preparative TLC (SiO₂: 10% MeOH in DCM) to afford compound 6d as a yellow amorphous solid (24 mg, 71%). ¹H NMR (400 MHz, DMSO-d₆) δ 13.09 (s, 1H), 7.78 (d, J=8.1 Hz, 3H), 7.57 (d, J=8.1 Hz, 3H), 7.33 (d, J=1.8 Hz, 1H), 7.28 (dd, J=7.8, 1.8 Hz, 1H), 7.21 (d, J=7.8 Hz, 1H), 7.11-7.04 (m, 1H), 6.90 (t, J=6.0 Hz, 1H), 6.76 (ddd, J=9.3, 2.4, 1.3 Hz, 1H), 6.56 (dt, J=11.8, 2.4 Hz, 1H), 4.32 (d, J=6.0 Hz, 2H), 2.23 (s, 3H). ¹³C NMR (101 MHz, DMSO-d₆) δ 166.8, 163.1 (d, J=239.9 Hz), 150.6 (d, J=11.0 Hz), 145.3, 139.3, 138.4, 134.8, 129.9 (2C), 129.6, 129.4, 127.4 (q, J=31.9 Hz), 125.1 (q, J=3.9 Hz, 2C), 125.0, 124.4 (q, J=271.8 Hz), 125.0, 109.8, 102.3 (d, J=23.5 Hz), 101.7 (d, J=25.7 Hz,), 45.9, 20.1. MSESI m/z: 402.1114 (C₂₂H₁₆F₄NO₂⁻ requires 402.1117).

The intermediate compound 4d was prepared as follows.

### a. Preparation of Compound 2-Methyl-4′-(trifluoromethyl)-[1,1′-biphenyl]-4-carbaldehyde (4d)

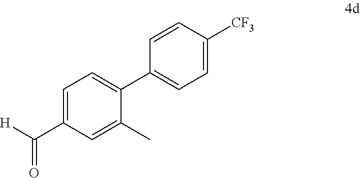 [SMILES: Cc1cc(C=O)ccc1-c1ccc(C(F)(F)F)cc1]

Using general procedure 2, employing 4-bromo-3-methylbenzaldehyde (38 mg, 0.19 mmol) and (4-(trifluoromethyl)phenyl)boronic acid (44 mg, 0.23 mmol), 3 hours, compound 4d was obtained after flash column chromatography (SiO₂, 0-15% EtOAc in hexanes) as a yellow amorphous solid (39 mg, 77%). ¹H NMR (400 MHz, CDCl₃) δ 10.05 (s, 1H), 7.85-7.80 (m, 1H), 7.78 (dd, J=7.8, 1.7 Hz, 1H), 7.75-7.66 (m, 1H), 7.49-7.41 (m, 2H), 7.38 (d, J=7.8 Hz, 1H), 2.34 (s, 3H).

### Example 12. Preparation of 3-(((2,2′-Dimethyl-4′-(trifluoromethyl)-[1,1′-biphenyl]-4-yl)methyl)amino)-5-fluorobenzoic Acid (6e)

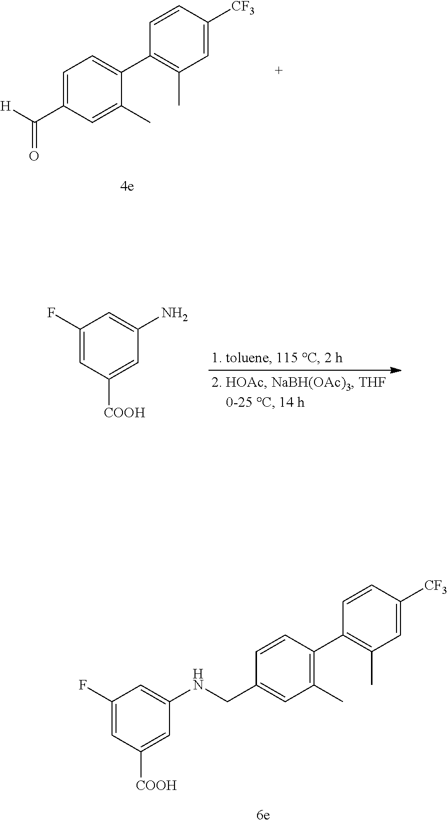 [SMILES: Cc1cc(C=O)ccc1-c1ccc(C(F)(F)F)cc1C ; Nc1cc(F)cc(C(=O)O)c1 ; Cc1cc(CNc2cc(F)cc(C(=O)O)c2)ccc1-c1ccc(C(F)(F)F)cc1C]

A mixture of 4e (31 mg, 0.11 mmol) and 3-amino-5-fluorobenzoic acid (17 mg, 0.11 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (47 mg, 0.22 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM) to afford compound 6d as a yellow amorphous solid (40 mg, 86%). ¹H NMR (400 MHz, DMSO-d₆) δ 12.98 (s, 1H), 7.68 (d, J=2.0 Hz, 1H), 7.57 (dd, J=7.9, 2.0 Hz, 1H), 7.32 (s, 1H), 7.31-7.21 (m, 2H), 7.13-7.09 (m, 1H), 7.06 (d, J=7.7 Hz, 1H), 6.89 (t, J=5.8 Hz, 1H), 6.77 (ddd, J=9.3, 2.5, 1.3 Hz, 1H), 6.60 (dt, J=11.8, 2.5 Hz, 1H), 4.33 (d, J=5.8 Hz, 2H), 2.07 (s, 3H), 1.98 (s, 3H). ¹³C NMR (101 MHz, DMSO-d₆) δ 166.8, 163.1 (d, J=240.2 Hz), 150.7 (d, J=11.4 Hz), 145.2, 138.9, 138.2, 137.0, 134.9, 133.2 (d, J=9.9 Hz), 130.1, 128.9, 128.8, 127.9 (q, J=31.5 Hz), 126.4 (q, J=3.8 Hz), 124.8, 124.4 (q, J=272.2 Hz), 122.4 (q, J=4.0 Hz), 109.8, 102.3 (d, J=23.5 Hz), 101.9 (d, J=25.3 Hz), 46.0, 19.5, 19.4. MSESI n z: 416.1297 (C₂₃H₁₈F₄NO₂⁻ requires 416.1274).

The intermediate compound 4e was prepared as follows.

### a. Preparation of 2,2′-Dimethyl-4′-(trifluoromethyl)-[1,1′-biphenyl]-4-carbaldehyde (4e)

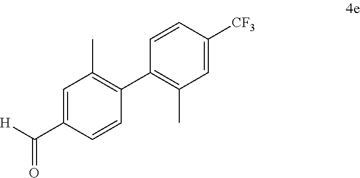 [SMILES: Cc1cc(C=O)ccc1-c1ccc(C(F)(F)F)cc1C]

Using general procedure 2, employing 4-bromo-3-methylbenzaldehyde (40 mg, 0.2 mmol) and (2-methyl-4-(trifluoromethyl)phenyl)boronic acid (49 mg, 0.24 mmol), 3 hours, compound 4e was obtained after flash column chromatography (SiO₂, 0-15% EtOAc in hexanes) as a yellow oil (33 mg, 59%). ¹H NMR (400 MHz, CDCl₃) δ 10.05 (s, 1H), 7.85-7.80 (m, 1H), 7.77 (dd, J=7.6, 1.7 Hz, 1H), 7.56 (s, 1H), 7.52 (d, J=7.9 Hz, 1H), 7.26 (d, J=7.6 Hz, 1H), 7.21 (d, J=7.9 Hz, 1H), 2.13 (s, 3H), 2.11 (s, 3H).

### Example 13. Preparation of 3-Fluoro-5-(((2′-methyl-4′-(trifluoromethyl)-[1,1′-biphenyl]-4-yl)methyl)amino)benzoic Acid (6f)

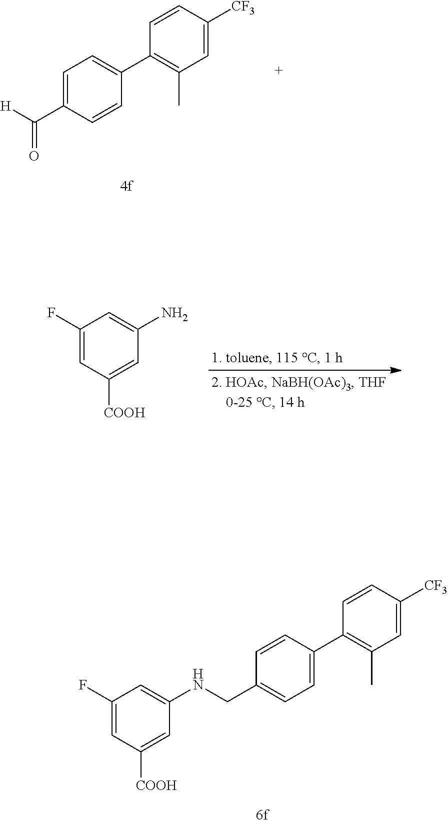 [SMILES: Cc1cc(C(F)(F)F)ccc1-c1ccc(C=O)cc1 ; Nc1cc(F)cc(C(=O)O)c1 ; Cc1cc(C(F)(F)F)ccc1-c1ccc(CNc2cc(F)cc(C(=O)O)c2)cc1]

A mixture of 4f (50 mg, 0.19 mmol) and 3-amino-5-fluorobenzoic acid (29 mg, 0.19 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (80 mg, 0.38 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM) to afford compound 6f as a colorless sticky amorphous solid (57 mg, 75%). ¹H NMR (400 MHz, DMSO-d₆) δ 7.66 (d, J=2.0 Hz, 1H), 7.57 (dd, J=8.1, 2.0 Hz, 1H), 7.46 (d, J=8.0 Hz, 2H), 7.40 (d, J=8.1 Hz, 1H), 7.35 (d, J=8.0 Hz, 2H), 7.11 (t, J=1.8 Hz, 1H), 6.93 (t, J=5.6 Hz, 1H), 6.82-6.72 (m, 1H), 6.60 (dt, J=11.8, 2.3 Hz, 1H), 4.38 (d, J=5.6 Hz, 2H), 2.30 (s, 3H). ¹³C NMR (101 MHz, DMSO-d₆) δ 166.9 (d, J=3.5 Hz), 163.2 (d, J=240.1 Hz), 150.7 (d, J=11.3 Hz), 145.2, 138.9, 138.4, 136.4, 133.5 (d, J=9.8 Hz), 130.4, 128.9 (2C), 127.8 (q, J=31.5 Hz), 127.3 (2C), 126.9 (q, J=3.8 Hz), 124.4 (q, J=272.1 Hz), 122.6 (q, J=3.8 Hz), 109.9, 102.4 (d, J=23.4 Hz), 101.9 (d, J=25.3 Hz), 46.0, 20.1. MSESI m/z: 402.1114 (C₂₂H₁₆F₄NO₂⁻ requires 402.1117).

The intermediate compound 4f was prepared as follows.

### a. Preparation of 2′-Methyl-4′-(trifluoromethyl)-[1,1′-biphenyl]-4-carbaldehyde (4f)

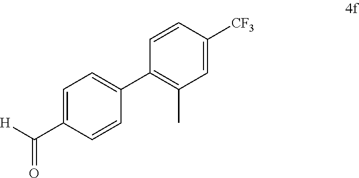 [SMILES: Cc1cc(C(F)(F)F)ccc1-c1ccc(C=O)cc1]

Using general procedure 2, employing 4-bromobenzaldehyde (37 mg, 0.2 mmol) and (2-methyl-4-(trifluoromethyl)phenyl)boronic acid (49 mg, 0.24 mmol), 3 hours, compound 4f was obtained after flash column chromatography (SiO₂, 0-15% EtOAc in hexanes) as a white amorphous solid (50 mg, 95%). ¹H NMR (400 MHz, CDCl₃) δ 10.09 (s, 1H), 7.97 (d, J=8.2 Hz, 2H), 7.56 (s, 1H), 7.53 (d, J=8.0 Hz, 1H), 7.49 (d, J=8.2 Hz, 2H), 7.34 (d, J=8.0 Hz, 1H), 2.32 (s, 3H).

### Example 14. Preparation of 3-(((4′-Cyano-[1,1′-biphenyl]-4-yl)methyl)amino)-5-fluorobenzoic Acid (6i)

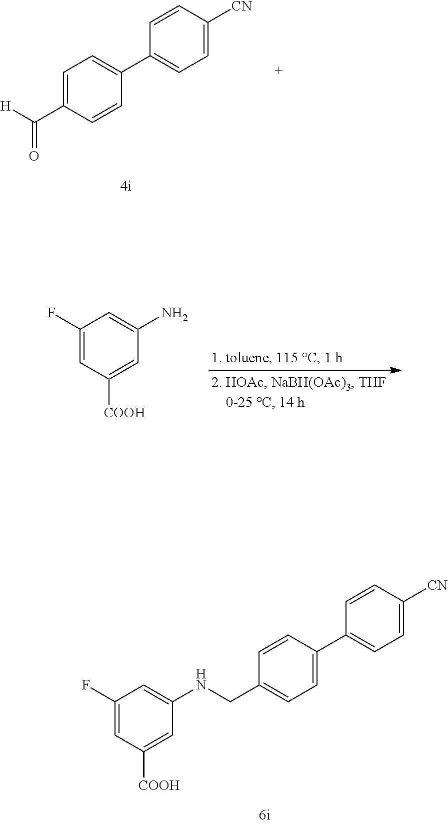 [SMILES: N#Cc1ccc(-c2ccc(C=O)cc2)cc1 ; Nc1cc(F)cc(C(=O)O)c1 ; N#Cc1ccc(-c2ccc(CNc3cc(F)cc(C(=O)O)c3)cc2)cc1]

A mixture of 4i (43 mg, 0.21 mmol) and 3-amino-5-fluorobenzoic acid (32 mg, 0.21 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (88 mg, 0.41 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM). The appropriate fractions were collected and concentrated under reduced pressure. The residue was washed with acetone (2 mL) to afford compound 6i as a yellow amorphous solid (36 mg, 50%). ¹H NMR (400 MHz, DMSO-d₆) δ 8.00-7.80 (m, 4H), 7.73 (d, J=8.1 Hz, 2H), 7.49 (d, J=8.1 Hz, 2H), 7.10-7.02 (m, 1H), 6.96 (t, J=5.9 Hz, 1H), 6.76 (dt, J=9.1, 1.9 Hz, 1H), 6.56 (dt, J=11.8, 2.3 Hz, 1H), 4.38 (d, J=5.9 Hz, 2H). ¹³C NMR (101 MHz, DMSO-d₆) δ 166.8, 163.1 (d, J=240.1 Hz), 150.5 (d, J=11.3 Hz), 144.4, 140.3, 136.8, 132.8, 127.9, 127.4, 127.2, 118.9, 109.9, 102.4 (d, J=23.1 Hz), 101.9 (d, J=25.4 Hz), 45.8. MSESI m/z: 345.1029 (C₂₁H₁₄FN₂O₂⁻ requires 345.1039).

The intermediate compound 4i was prepared as follows.

### a. Preparation of 4′-Formyl-[1,1′-biphenyl]-4-carbonitrile (4i)

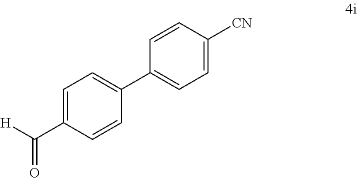 [SMILES: N#Cc1ccc(-c2ccc(C=O)cc2)cc1]

Using general procedure 2, employing 4-bromobenzaldehyde (37 mg, 0.2 mmol) and (4-cyanophenyl)boronic acid (35 mg, 0.24 mmol), 3 hours, compound 4i was obtained after flash column chromatography (SiO₂, 0-20% EtOAc in hexanes) as a white amorphous solid (40 mg, 97%). ¹H NMR (400 MHz, CDCl₃) δ 10.09 (s, 1H), 8.00 (d, J=8.3 Hz, 1H), 7.87 (d, J=2.2 Hz, 1H), 7.83-7.80 (m, 1H), 7.79-7.75 (m, 2H), 7.68 (d, J=7.9 Hz, 1H), 7.61 (t, J=7.9 Hz, 1H).

### Example 15. Preparation of 3-Fluoro-5-(((4′-(trifluoromethoxy)-[1,1′-biphenyl]-4-yl)methyl)amino)benzoic acid (6j)

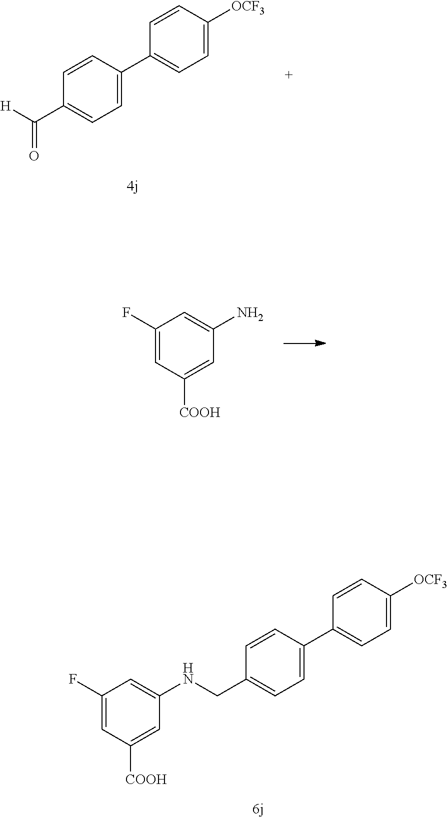 [SMILES: O=Cc1ccc(-c2ccc(OC(F)(F)F)cc2)cc1 ; Nc1cc(F)cc(C(=O)O)c1 ; O=C(O)c1cc(F)cc(NCc2ccc(-c3ccc(OC(F)(F)F)cc3)cc2)c1]

A mixture of 4j (30 mg, 0.11 mmol) and 3-amino-5-fluorobenzoic acid (17 mg, 0.11 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (48 mg, 0.23 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6j as a yellow amorphous solid (35 mg, 57%). ¹H NMR (400 MHz, DMSO-d₆) δ 7.82-7.73 (m, 2H), 7.69-7.62 (m, 2H), 7.49-7.40 (m, 4H), 7.08 (s, 1H), 6.93 (t, J=6.0 Hz, 1H), 6.80-6.72 (m, 1H), 6.55 (dt, J=11.8, 2.3 Hz, 1H), 4.36 (d, J=6.0 Hz, 2H). ¹³C NMR (101 MHz, DMSO-d₆) δ 166.9, 163.1 (d, J=240.3 Hz), 150.5 (d, J=11.4 Hz), 147.7, 139.2 (d, J=8.1 Hz), 137.3, 128.4 (2C), 127.8 (2C), 127.1, 126.9 (2C), 126.5, 121.4 (2C), 118.9, 109.9, 102.3 (d, J=23.5 Hz), 101.9 (d, J=25.3 Hz), 45.8. MSESI m/z: 404.0918 (C₂₁H₁₄F₄NO₃⁻ requires 404.0910).

The intermediate compound 4j was prepared as follows.

### a. Preparation of 4′-(Trifluoromethoxy)-[1,1′-biphenyl]-4-carbaldehyde (4j)

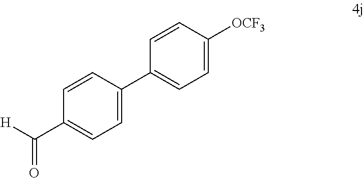 [SMILES: O=Cc1ccc(-c2ccc(OC(F)(F)F)cc2)cc1]

Using general procedure 2, employing 4-bromobenzaldehyde (50 mg, 0.27 mmol) and (4-(trifluoromethoxy)phenyl)boronic acid, 4 hours, compound 4j was obtained after flash column chromatography (SiO₂, 0-20% EtOAc in hexanes) (70 mg, 97%). ¹H NMR (400 MHz, CDCl₃) δ 10.07 (s, 1H), 7.99-7.94 (m, 2H), 7.77-7.70 (m, 2H), 7.69-7.60 (m, 2H), 7.38-7.30 (m, 2H).

### Example 16. Preparation of 3-Fluoro-5-(((4′-methoxy-[1,1′-biphenyl]-4-yl)methyl)amino)benzoic acid (6k)

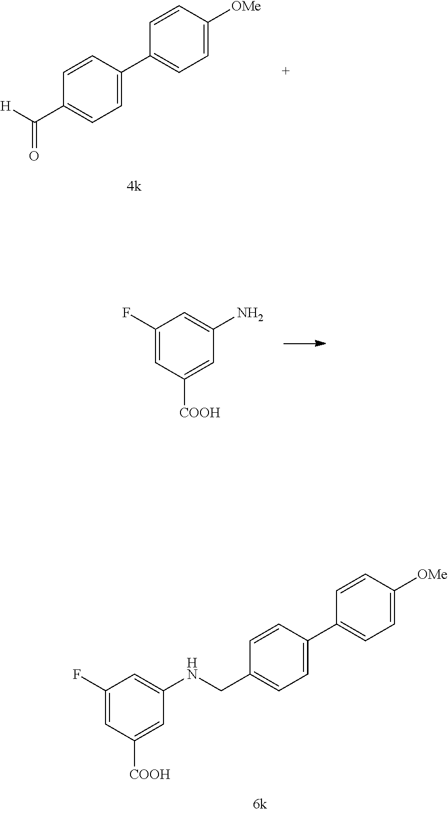 [SMILES: COc1ccc(-c2ccc(C=O)cc2)cc1 ; Nc1cc(F)cc(C(=O)O)c1 ; COc1ccc(-c2ccc(CNc3cc(F)cc(C(=O)O)c3)cc2)cc1]

A mixture of 4k (25 mg, 0.12 mmol) and 3-amino-5-fluorobenzoic acid (18 mg, 0.12 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (50 mg, 0.24 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6k as a yellow amorphous solid (24 mg, 58%). ¹H NMR (400 MHz, DMSO-d₆) δ 7.58 (dd, J=8.6, 2.9 Hz, 4H), 7.40 (d, J=8.2 Hz, 2H), 7.10-7.05 (m, 1H), 7.04-6.98 (m, 2H), 6.88 (t, J=6.1 Hz, 1H), 6.79-6.73 (m, 1H), 6.54 (dt, J=12.0, 2.4 Hz, 1H), 4.33 (d, J=6.0 Hz, 2H), 3.79 (s, 3H). ¹³C NMR (101 MHz, DMSO-d₆) δ 166.9, 163.1 (d, J=240.3 Hz), 158.8, 150.5 (d, J=11.0 Hz), 138.4, 137.8, 132.3, 127.7 (2C), 127.6 (2C), 126.2 (2C), 114.3 (2C), 109.9, 102.3 (d, J=23.5 Hz), 101.8 (d, J=25.3 Hz), 55.1, 45.9. MSESI m/z: 350.1192 (C₂₁H₁₇FNO₃⁻ requires 350.1192).

The intermediate compound 4k was prepared as follows.

### a. Preparation of 4′-Methoxy-[1,1′-biphenyl]-4-carbaldehyde (4k)

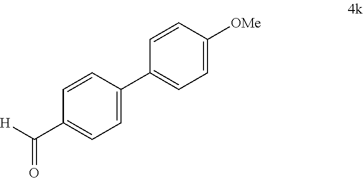 [SMILES: COc1ccc(-c2ccc(C=O)cc2)cc1]

Using general procedure 2, employing 4-bromobenzaldehyde (50 mg, 0.27 mmol) and (4-methoxyphenyl)boronic acid (49 mg, 0.32 mmol), 4 hours, compound 4k was obtained after flash column chromatography (SiO₂, 0-20% EtOAc in hexanes) (56 mg, 98%). ¹H NMR (400 MHz, CDCl₃) δ 10.04 (s, 1H), 7.96-7.90 (m, 2H), 7.75-7.69 (m, 2H), 7.63-7.57 (m, 2H), 7.06-6.99 (m, 2H), 3.87 (s, 3H).

### Example 17. Preparation of 3-Fluoro-5-(((4′-fluoro-[1,1′-biphenyl]-4-yl)methyl)amino)benzoic Acid (6l)

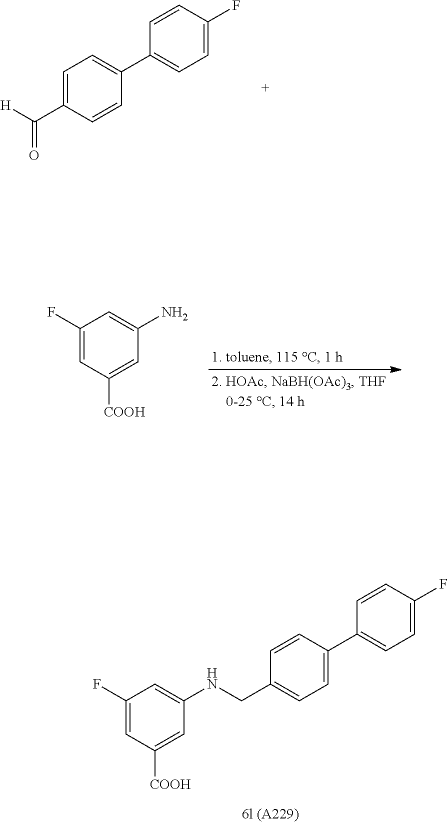 [SMILES: O=Cc1ccc(-c2ccc(F)cc2)cc1 ; Nc1cc(F)cc(C(=O)O)c1 ; O=C(O)c1cc(F)cc(NCc2ccc(-c3ccc(F)cc3)cc2)c1]

A mixture of 4′-fluoro-[1,1′-biphenyl]-4-carbaldehyde (204 mg, 1.02 mmol) and 3-amino-5-fluorobenzoic acid (155 mg, 1 mmol) in toluene (10 mL) was refluxed using a Dean-Stark apparatus at 130° C. for 2 hours. The solvent was removed under reduced pressure by rotary evaporation. The residue was dissolved in THF (10 mL) and cooled to 0° C. Sodium triacetoxyborohydride (424 mg, 2 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The residue was crystallized with EtOH to give 6l as a yellow solid (159 mg, 47%). ¹H NMR (400 MHz, DMSO-d₆) δ 12.95 (s, 1H), 7.72-7.66 (m, 2H), 7.62 (d, J=8.2 Hz, 1H), 7.43 (d, J=8.2 Hz, 2H), 7.27 (t, J=8.9 Hz, 2H), 7.10-7.06 (m, 1H), 6.94 (t, J=5.9 Hz, 1H), 6.77 (ddd, J=9.3, 2.4, 1.3 Hz, 1H), 6.57 (dt, J=11.8, 2.3 Hz, 1H), 4.35 (d, J=5.9 Hz, 2H). ¹³C NMR (101 MHz, DMSO-d₆) δ 166.8 (d, J=3.5 Hz), 163.1 (d, J=240.2 Hz), 161.8 (d, J=244.2 Hz), 150.6 (d, J=11.3 Hz), 138.6, 137.7, 136.4 (d, J=3.0 Hz), 133.1 (d, J=9.9 Hz), 128.5 (d, J=8.2 Hz, 2C), 127.8 (2C), 126.7 (2C), 115.7 (d, J=21.3 Hz, 2C), 109.9, 102.3 (d, J=23.8 Hz), 102.1 (d, J=25.7 Hz), 45.8. MSESI m/z: 338.0988 (C₂₀H₁₄F₂NO₂⁻ requires 338.0998).

### Example 18. Preparation of 3-(((4′-Chloro-[1,1′-biphenyl]-4-yl)methyl)amino)-5-fluorobenzoic acid (6m)

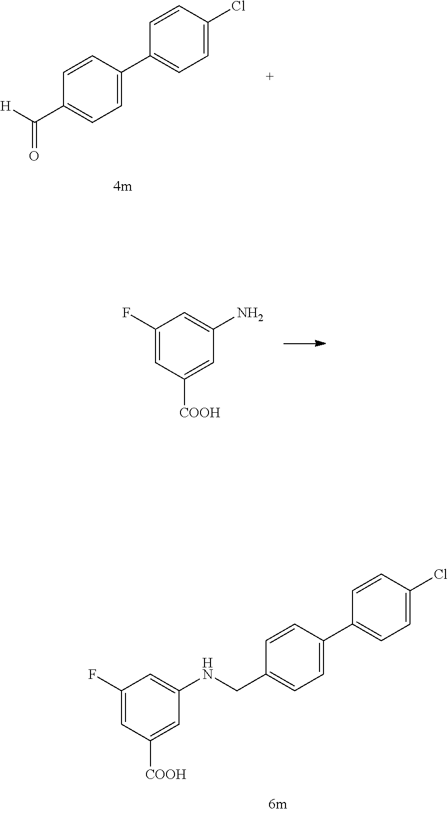 [SMILES: O=Cc1ccc(-c2ccc(Cl)cc2)cc1 ; Nc1cc(F)cc(C(=O)O)c1 ; O=C(O)c1cc(F)cc(NCc2ccc(-c3ccc(Cl)cc3)cc2)c1]

A mixture of 4m (20 mg, 0.092 mmol) and 3-amino-5-fluorobenzoic acid (14 mg, 0.092 mmol) in toluene (2 mL) was stirred at 15° C. for 1 hour. The solvent was removed under reduced pressure by rotary evaporation. The residue was dissolved in THF (2 mL) and cooled to 0° C. Sodium triacetoxy-borohydride (39 mg, 0.18 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6m as a yellow amorphous solid (27 mg, 82%). ¹H NMR (400 MHz, DMSO-d₆) δ 13.00 (s, 1H), 7.71-7.66 (m, 2H), 7.66-7.62 (m, 2H), 7.53-7.47 (m, 2H), 7.47-7.42 ((m, 2H), 7.10-7.05 (m, 1H), 6.94 (t, J=6.1 Hz, 1H), 6.76 (ddd, J=9.3, 2.4, 1.4 Hz, 1H), 6.56 (dt, J=11.7, 2.3 Hz, 1H), 4.35 (d, J=6.0 Hz, 2H). ¹³C NMR (101 MHz, DMSO-d₆) 166.8, 163.1 (d, J=240.3 Hz), 150.6 (d, J=11.0 Hz), 139.1, 138.7, 137.4, 133.3 (d, J=8.8 Hz), 132.2, 128.8 (2C), 128.3 (2C), 127.8 (2C), 126.7 (2C), 109.9, 102.3 (d, J=23.5 Hz), 102.0 (d, J=25.7 Hz), 45.8. MSESI m/z: 354.0694 (C₂₀H₁₄ClFNO₂⁻ requires 354.0703)

The intermediate compound 4m was prepared as follows.

### a. Preparation of 4′-Chloro-[1,1′-biphenyl]-4-carbaldehyde (4m)

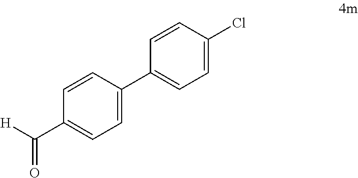 [SMILES: O=Cc1ccc(-c2ccc(Cl)cc2)cc1]

Using general procedure 2, employing 4-bromobenzaldehyde (200 mg, 1.08 mmol) and (4-chlorophenyl)boronic acid (203 mg, 1.3 mmol), 4 hours, compound 4m was obtained after flash column chromatography (SiO₂, 0-20% EtOAc in hexanes) (63 mg, 27%). ¹H NMR (400 MHz, CDCl₃) δ 9.99 (s, 1H), 7.93-7.85 (m, 2H), 7.68-7.62 (m, 2H), 7.53-7.47 (m, 2H), 7.43-7.36 (m, 2H).

### Example 19. Preparation of 3-Fluoro-5-(((4′-methyl-[1,1′-biphenyl]-4-yl)methyl)amino)benzoic acid (6n)

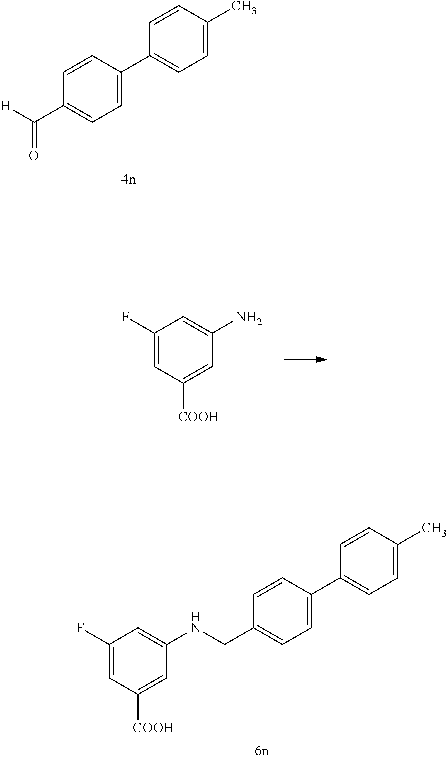 [SMILES: Cc1ccc(-c2ccc(C=O)cc2)cc1 ; Nc1cc(F)cc(C(=O)O)c1 ; Cc1ccc(-c2ccc(CNc3cc(F)cc(C(=O)O)c3)cc2)cc1]

A mixture of 4n (25 mg, 0.13 mmol) and 3-amino-5-fluorobenzoic acid (20 mg, 0.13 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (54 mg, 0.25 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6m as a yellow amorphous solid (38 mg, 89%). ¹H NMR (400 MHz, DMSO-d₆) δ 7.63-7.58 (m, 2H), 7.56-7.51 (m, 2H), 7.45-7.39 (m, 2H), 7.25 (d, J=8.3 Hz, 2H), 7.10-7.06 (m, 1H), 6.91 (t, J=6.4 Hz, 1H), 6.76 (ddd, J=9.3, 2.4, 1.3 Hz, 1H), 6.55 (dt, J=11.8, 2.3 Hz, 1H), 4.34 (d, J=5.9 Hz, 2H), 2.33 (s, 3H). ¹³C NMR (101 MHz, DMSO-d₆) δ 166.8, 163.1 (d, J=240.3 Hz), 150.6 (d, J=11.0 Hz), 138.7, 138.3, 137.0, 136.6, 133.4, 129.5 (2C), 127.7 (2C), 126.4 (2C), 126.4 (2C), 109.9, 102.3 (d, J=23.5 Hz), 101.9 (d, J=25.7 Hz), 45.9, 20.7. MSESI m/z: 334.1250 (C₂₁H₁₇FNO₂⁻ requires 334.1243).

The intermediate compound 4n was prepared as follows.

### a. Preparation of 4′-Methyl-[1,1′-biphenyl]-4-carbaldehyde (4n)

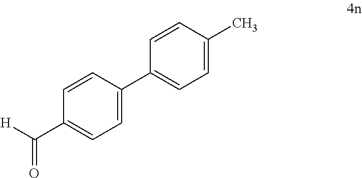

Using general procedure 2, employing 4-bromobenzaldehyde (50 mg, 0.27 mmol) and p-tolylboronic acid (44 mg, 0.32 mmol), 4 hours, compound 4n was obtained after flash column chromatography (SiO₂, 0-20% EtOAc in hexanes) (37 mg, 70%). ¹H NMR (400 MHz, CDCl₃) δ 10.05 (s, 1H), 7.97-7.89 (m, 2H), 7.79-7.71 (m, 2H), 7.58-7.51 (m, 2H), 7.32-7.27 (m, 2H).

### Example 20. Preparation of 3-(((4′-Cyclopentyl-[1,1′-biphenyl]-4-yl)methyl)amino)-5-fluorobenzoic acid (60)

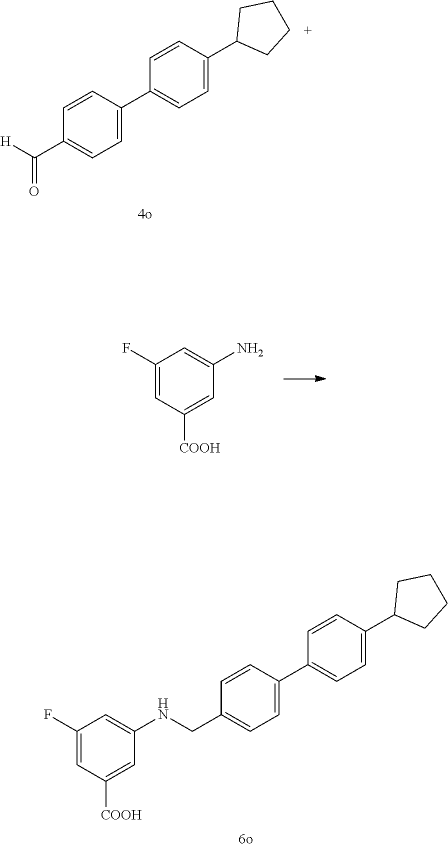 [SMILES: O=Cc1ccc(-c2ccc(C3CCCC3)cc2)cc1 ; Nc1cc(F)cc(C(=O)O)c1 ; O=C(O)c1cc(F)cc(NCc2ccc(-c3ccc(C4CCCC4)cc3)cc2)c1]

A mixture of 4o (25 mg, 0.10 mmol) and 3-amino-5-fluorobenzoic acid (15 mg, 0.10 mmol) in toluene (2 mL) was stirred at 115° C. for 1 hour. The solvent was removed under reduced pressure by rotary evaporation. The residue was dissolved in THF (2 mL) and cooled to 0° C. Sodium triacetoxy-borohydride (42 mg, 0.2 mmol) was added and the mixture was stirred at 0° C. for 15 minutes. Acetic acid (1 μL) was added and the mixture was then warmed to room temperature and stirred. After the reaction was judged to be completed by TLC (16 hours), it was quenched with water and extracted with EtOAc. The organic layer was washed with water and brine and concentrated under reduced pressure by rotary evaporation. The crude residue was purified by flash column chromatography (SiO₂: 0-8% MeOH in DCM with 1% formic acid) to afford compound 6o as a yellow amorphous solid (38 mg, 89%). ¹H NMR (400 MHz, DMSO-d₆) δ 7.64-7.58 (m, 2H), 7.58-7.53 (m, 2H), 7.42 (d, J=8.3 Hz, 2H), 7.34-7.29 (m, 2H), 7.10-7.06 (m, 1H), 6.91 (t, J=5.6 Hz, 1H), 6.76 (dt, J=9.3, 2.4 Hz, 1H), 6.56 (dt, J=11.8, 2.3 Hz, 1H), 4.34 (d, J=6.0 Hz, 2H), 2.99 (tt, J=9.9, 7.3 Hz, 1H), 2.10-1.97 (m, 2H), 1.84-1.71 (m, 2H), 1.71-1.61 (m, 2H), 1.61-1.49 (m, 2H). ¹³C NMR (101 MHz, DMSO-d₆) δ 166.8, 163.1 (d, 0.1=240.3 Hz), 150.6 (d, J=11.0 Hz), 145.1, 138.8, 138.3, 137.4, 133.4, 127.8 (2C), 127.5 (2C), 126.5 (2C), 126.4 (2C), 109.9, 102.3 (d, J=23.1 Hz), 101.9 (d, J=25.7 Hz), 45.9, 45.0, 34.2 (2C), 25.0 (2C). MSESI m/z: 388.1712 (C₂₅H₂₃FNO₂⁻ requires 388.1713).

The intermediate compound 4o was prepared as follows.

### a. Preparation of 4′-Cyclopentyl-[1,1′-biphenyl]-4-carbaldehyde (4o)

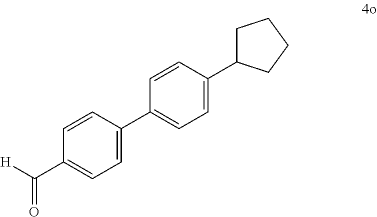 [SMILES: O=Cc1ccc(-c2ccc(C3CCCC3)cc2)cc1]

Using general procedure 2, employing 4-bromobenzaldehyde (50 mg, 0.27 mmol) and (4-cyclopentylphenyl)boronic acid (62 mg, 0.32 mmol), 4 hours, compound 4o was obtained after flash column chromatography (SiO₂, 0-20% EtOAc in hexanes) (40 mg, 59%). ¹H NMR (400 MHz, CDCl₃) δ 10.05 (d, J=2.4 Hz, 1H), 7.98-7.91 (m, 2H), 7.75 (dd, J=8.3, 2.3 Hz, 2H), 7.58 (dd, J=8.4, 2.3 Hz, 2H), 7.36 (dd, J=8.3, 2.3 Hz, 2H), 3.13-3.00 (m, 1H), 2.13 (d, J=7.4 Hz, 2H), 1.92-1.80 (m, 2H), 1.78-1.58 (m, 4H).

### Example 21. Preparation of 3-Fluoro-5-(((3-methoxy-4′-(trifluoromethyl)-[1,1′-biphenyl]-4-yl)methyl)amino)benzoic Acid (6p)

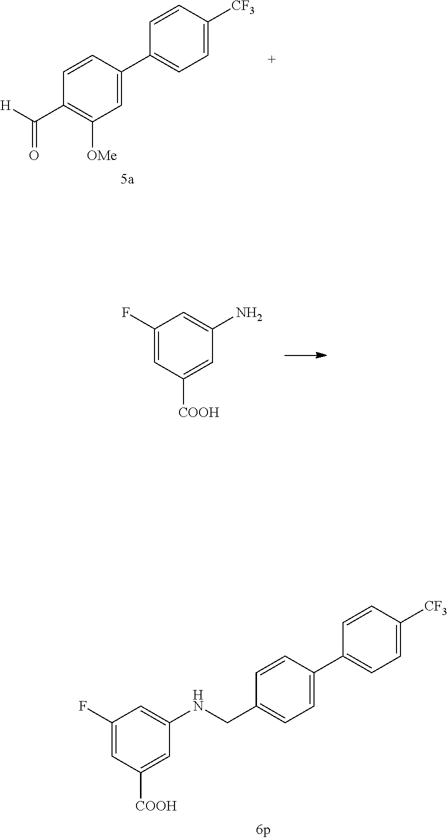 [SMILES: COc1cc(-c2ccc(C(F)(F)F)cc2)ccc1C=O ; Nc1cc(F)cc(C(=O)O)c1 ; O=C(O)c1cc(F)cc(NCc2ccc(-c3ccc(C(F)(F)F)cc3)cc2)c1]

A mixture of 5a (37 mg, 0.13 mmol) and 3-amino-5-fluorobenzoic acid (20 mg, 0.13 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (56 mg, 0.26 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-10% MeOH in DCM) to afford compound 6p as a white amorphous solid (38 mg, 69%). ¹H NMR (400 MHz, DMSO-d₆) δ 7.92 (d, J=8.2 Hz, 2H), 7.80 (d, J=8.2 Hz, 2H), 7.38-7.30 (m, 2H), 7.27 (dd, J=7.7, 1.7 Hz, 1H), 7.10-7.02 (m, 1H), 6.82-6.71 (m, 2H), 6.54 (dt, J=11.7, 2.4 Hz, 1H), 4.30 (d, J=5.8 Hz, 2H), 3.96 (s, 3H). ¹³C NMR (101 MHz, DMSO-d₆) δ 166.8, 163.1 (d, J=240.2 Hz), 157.4, 150.7 (d, J=11.4 Hz), 144.2, 138.7, 133.4 (d, J=9.5 Hz), 128.4, 127.8 (q, J=32.0 Hz), 127.6 (2C), 127.0, 125.7 (q, 0.1=3.7 Hz, 2C), 124.4 (q, J=271.8 Hz), 119.0, 109.5, 102.3 (d, J=23.4 Hz), 101.9 (d, J=25.6 Hz), 55.6, 40.9. MSESI m/z: 418.1078 (C₂₂H₁₆FNO₃⁻ requires 418.1066).

The intermediate compound 5a was prepared as follows.

### a. Preparation of 3-Methoxy-4′-(trifluoromethyl)-[1,1′-biphenyl]-4-carbaldehyde (5a)

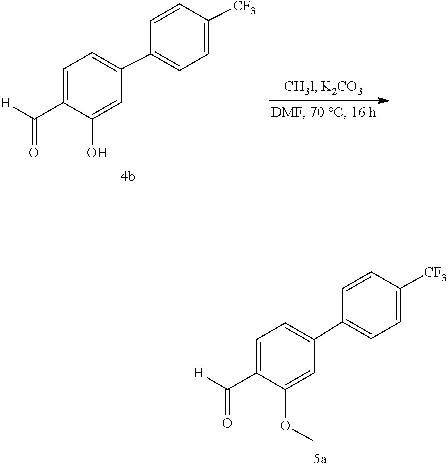 [SMILES: O=Cc1ccc(-c2ccc(C(F)(F)F)cc2)cc1O ; COc1cc(-c2ccc(C(F)(F)F)cc2)ccc1C=O]

A mixture of compound 4b (39 mg, 0.15 mmol), methyl iodide (10 μL, 0.16 mmol), and potassium carbonate (40 mg, 0.29 mmol) in DMF (1 mL) was stirred at 70° C. TLC showed compound 5a had the same Rf as compound 4b. Another methyl iodide (10 μL, 0.16 mmol) was added to make the reaction completed and the mixture was stirred at 70° C. for 16 h. The reaction was cooled to room temperature, quenched with water, and extracted with EtOAc/hexanes (v/v=1:1). The organic layer was washed with water and brine and concentrated under reduced pressure by rotary evaporation. The crude residue was purified by flash column chromatography (SiO₂, 0-30% EtOAc/hexanes) to give compound 5a as a white amorphous solid (37 mg, 90%). ¹H NMR (400 MHz, CDCl₃) δ 10.53 (s, 1H), 7.95 (d, J=8.0 Hz, 1H), 7.77-7.73 (m, 4H), 7.32-7.24 (m, 1H), 7.19 (d, J=1.5 Hz, 1H), 4.04 (s, 3H).

### Example 22. Preparation of 3-Fluoro-5-(((5-(4-(trifluoromethyl)phenyl)pyridin-2-yl)methyl)amino)benzoic Acid (8a)

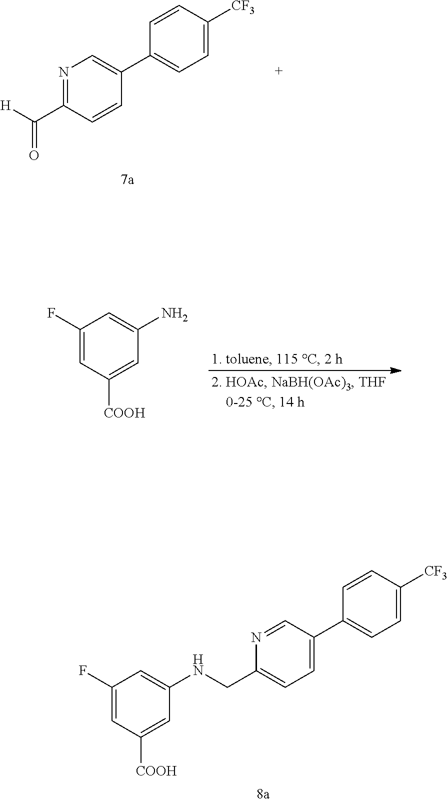 [SMILES: O=Cc1ccc(-c2ccc(C(F)(F)F)cc2)cn1 ; Nc1cc(F)cc(C(=O)O)c1 ; O=C(O)c1cc(F)cc(NCc2ccc(-c3ccc(C(F)(F)F)cc3)cn2)c1]

A mixture of 7a (20 mg, 0.08 mmol) and 3-amino-5-fluorobenzoic acid (12 mg, 0.08 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (34 mg, 0.16 mmol) was added and the mixture was stirred at 0° C. for 15 minutes. Acetic acid (1 μL) was added and the mixture was then warmed to room temperature and stirred After the reaction was judged to be completed by TLC (14 h), it was quenched with water and extracted with EtOAc. The organic layer was washed with water and brine and concentrated under reduced pressure by rotary evaporation. The crude residue was purified by flash column chromatography (SiO₂: 0-10% MeOH in DCM) to afford compound 8a as a white amorphous solid (10 mg, 32%). ¹H NMR (400 MHz, DMSO-d₆) δ 13.03 (s, 1H), 8.93 (d, J=2.3 Hz, 1H), 8.14 (dd, J=8.1, 2.3 Hz, 1H), 7.96 (d, J=8.2 Hz, 2H), 7.84 (d, J=8.2 Hz, 2H), 7.49 (d, J=8.1 Hz, 1H), 7.09 (s, 1H), 7.04 (t, J=6.1 Hz, 1H), 6.84-6.70 (m, 1H), 6.59 (dt, J=11.9, 2.3 Hz, 1H), 4.48 (d, J=6.1 Hz, 2H). ¹³C NMR (101 MHz, DMSO-d₆) δ 166.8, 163.1 (d, J=240.2 Hz), 159.1, 150.4 (d, J=11.21 Hz), 147.4, 141.1, 135.3, 133.7, 132.5, 128.3 (q, J=31.7 Hz), 127.6, 125.9 (q, J=3.8 Hz), 124.3 (q, J=271.8 Hz), 121.3, 109.8, 102.6 (d, J=23.6 Hz), 102.0 (d, J=26.2 Hz), 48.0. MSESI m/z: 389.0911 (C₂₀H₁₃F₄N₂O₂⁻ requires 389.0913).

The intermediate compound 7a was prepared as follows.

### a. Preparation of 6-(4-(Trifluoromethyl)phenyl)nicotinaldehyde (7a)

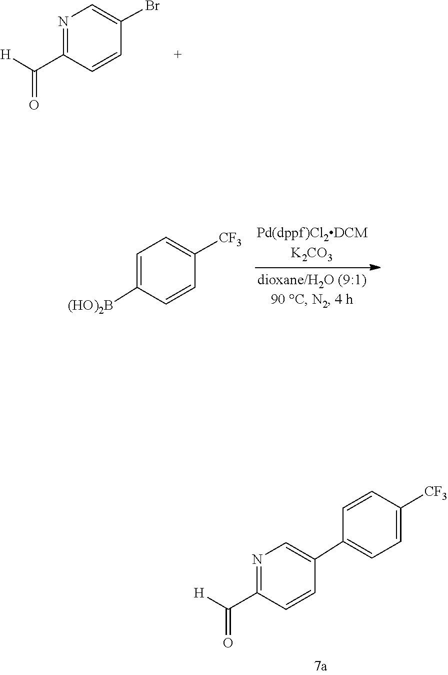 [SMILES: O=Cc1ccc(Br)cn1 ; OB(O)c1ccc(C(F)(F)F)cc1 ; O=Cc1ccc(-c2ccc(C(F)(F)F)cc2)cn1]

A mixture of 6-bromonicotinaldehyde (37 mg, 0.2 mmol), (4-(trifluoromethyl)phenyl)boronic acid (45 mg, 0.24 mmol), potassium carbonate (110 mg, 0.8 mmol), and Pd(dppf)Cl₂·CH₂Cl₂ (16 mg, 0.02 mmol) in dioxane/H₂O (v/v=9:1, 3 mL) was stirred at 90° C. under a nitrogen atmosphere for 3 hours. The reaction was cooled to room temperature, quenched with water, and extracted with EtOAc. The organic layer was washed with water and brine and concentrated under reduced pressure by rotary evaporation. The crude residue was purified by flash column chromatography (SiO₂: 0-25% EtOAc/hexanes) to give compound 7a as a white amorphous solid (46 mg, 92%). ¹H NMR (400 MHz, CDCl₃) δ 10.18 (s, 1H), 9.17 (d, 0.1=2.1 Hz, 1H), 8.29 (dd, J=8.2, 2.1 Hz, 1H), 8.25-8.15 (m, 2H), 7.96 (d, J=8.2 Hz, 1H), 7.78 (d, J=8.2 Hz, 2H).

### Example 23. Preparation of 3-Fluoro-5-(((6-(4-(trifluoromethyl)phenyl)pyridin-3-yl)methyl)amino)benzoic Acid (8b)

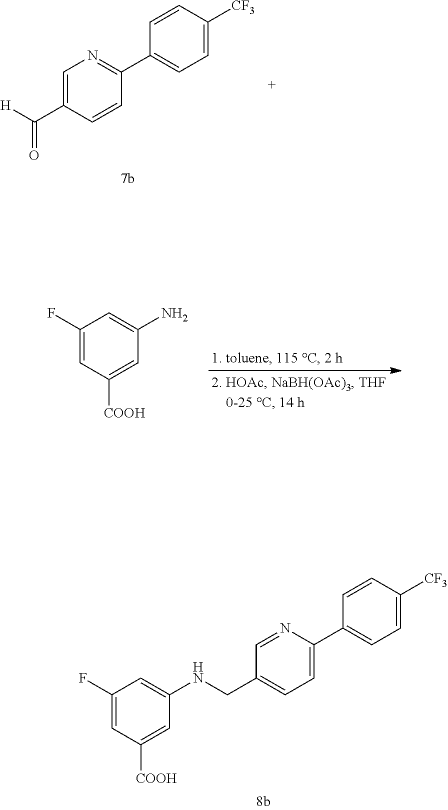 [SMILES: O=Cc1ccc(-c2ccc(C(F)(F)F)cc2)nc1 ; Nc1cc(F)cc(C(=O)O)c1 ; O=C(O)c1cc(F)cc(NCc2ccc(-c3ccc(C(F)(F)F)cc3)nc2)c1]

A mixture of 7b (26 mg, 0.1 mmol) and 3-amino-5-fluorobenzoic acid (16 mg, 0.1 mmol) in toluene (1 mL) was stirred at 115° C. for 2 hours. The solvent was removed under reduced pressure by rotary evaporation. The residue was dissolved in THF (1 mL) and cooled to 0° C. Sodium triacetoxy-borohydride (44 mg, 0.21 mmol) was added and the mixture was stirred at 0° C. for 15 minutes. Acetic acid (1 μL) was added and the mixture was then warmed to room temperature and stirred. After the reaction was judged to be completed by TLC (14 hours), it was quenched with water and extracted with EtOAc. The organic layer was washed with water and brine and concentrated under reduced pressure by rotary evaporation. The crude residue was purified by flash column chromatography (SiO₂: 0-10% MeOH in DCM with 1% formic acid) followed by preparative TLC (SiO₂: 15% MeOH in DCM) to afford compound 8b as a white amorphous solid (14 mg, 35%). ¹H NMR (400 MHz, DMSO-d₆) δ 8.72 (d, J=2.2 Hz, 1H), 8.29 (d, J=8.1 Hz, 2H), 8.05 (d, J=8.1 Hz, 1H), 7.89 (dd, J=8.1, 2.2 Hz, 1H), 7.83 (d, J=8.1 Hz, 2H), 7.09 (s, 1H), 6.94 (t, J=5.7 Hz, 1H), 6.84-6.70 (m, 1H), 6.61 (dd, J=11.8, 2.3 Hz, 1H), 4.42 (d, J=5.7 Hz, 2H). ¹³C NMR (101 MHz, DMSO-d₆) δ 166.9, 163.1 (d, J=240.3 Hz), 153.1, 150.2 (d, J=11.3 Hz), 149.1, 142.3, 136.4, 134.8, 134.1, 129.0 (q, J=31.8 Hz), 127.1 (2C), 125.6 (q, J=3.8 Hz, 2C), 124.3 (q, J=272.0 Hz), 120.7, 110.0, 102.7 (d, J=23.4 Hz), 101.9 (d, J=25.4 Hz), 43.6. MSESI m/z: 389.0933 (C₂₀H₁₃F₄N₂O₂⁻ requires 389.0913).

The intermediate compound 7b was prepared as follows.

### a. Preparation of 5-(4-(Trifluoromethyl)phenyl)picolinaldehyde (7b)

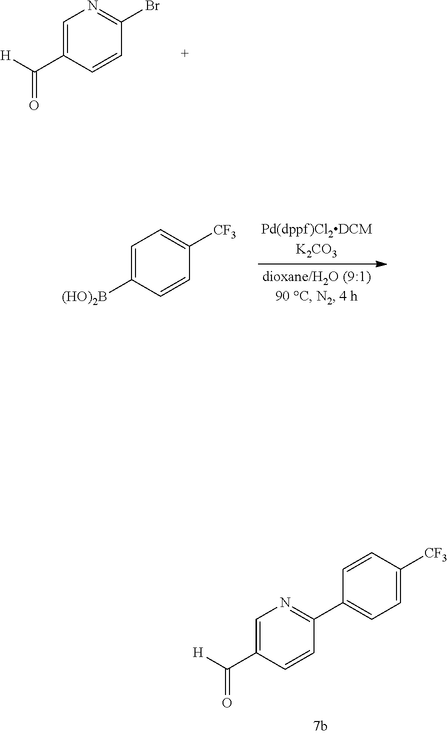 [SMILES: O=Cc1ccc(Br)nc1 ; OB(O)c1ccc(C(F)(F)F)cc1 ; O=Cc1ccc(-c2ccc(C(F)(F)F)cc2)nc1]

A mixture of 5-bromo-2-picolinaldehyde (37 mg, 0.2 mmol), (4-(trifluoromethyl)phenyl)boronic acid (45 mg, 0.24 mmol), potassium carbonate (110 mg, 0.8 mmol), and Pd(dppf)Cl₂·CH₂Cl₂ (16 mg, 0.02 mmol) in dioxane/H₂O (v/v=9:1, 3 mL) was stirred at 90° C. under a nitrogen atmosphere for 3 hours. The reaction was cooled to room temperature, quenched with water and extracted with EtOAc. The organic layer was washed with water and brine and concentrated under reduced pressure by rotary evaporation. The crude residue was purified by flash column chromatography (SiO₂: 0-25% EtOAc/hexanes) to give compound 7b as a white amorphous solid (20 mg, 40%). ¹H NMR (400 MHz, CDCl₃) δ 10.15 (s, 1H), 9.03 (t, J=1.5 Hz, 1H), 8.08 (d, J=1.5 Hz, 2H), 7.83-7.73 (m, 4H).

### Example 24. Preparation of 3-fluoro-5-(((2-(4-(trifluoromethyl)phenyl)thiazol-5-yl)methyl)amino)benzoic Acid (8c)

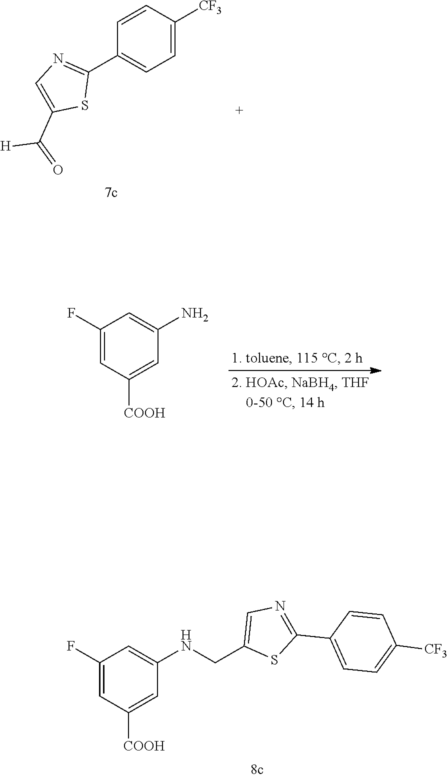 [SMILES: O=Cc1cnc(-c2ccc(C(F)(F)F)cc2)s1 ; Nc1cc(F)cc(C(=O)O)c1 ; O=C(O)c1cc(F)cc(NCc2cnc(-c3ccc(C(F)(F)F)cc3)s2)c1]

A mixture of 7c (17 mg, 0.064 mmol) and 3-amino-5-fluorobenzoic acid (10 mg, 0.064 mmol) in toluene (2 mL) was stirred at 115° C. for 2 hours. The solvent was removed under reduced pressure by rotary evaporation. The residue was dissolved in THF (1 mL) and cooled to 0° C. Sodium borohydride (4.9 mg, 0.13 mmol) was added, and the mixture was stirred at 0° C. for 15 min. Acetic acid (1 μL) was added and the mixture was then warmed to 50° C. and stirred. After the reaction was judged to be completed by TLC (14 h), it was quenched with water and extracted with EtOAc. The organic layer was washed with water and brine and concentrated under reduced pressure by rotary evaporation. The crude residue was purified by flash column chromatography (SiO₂: 0-10% MeOH in DCM with 1% formic acid) to afford compound 8c as a yellow amorphous solid (9 mg, 40%). ¹H NMR (400 MHz, Acetone-d&) δ 8.15 (d, J=8.2 Hz, 2H), 7.94 (s, 1H), 7.81 (d, J=8.3 Hz, 2H), 7.28 (t, J=2.2 Hz, 1H), 6.97 (dt, J=9.1, 2.2 Hz, 1H), 6.75 (dt, J=11.4, 2.2 Hz, 1H), 6.30 (t, J=5.0 Hz, 1H), 4.79 (d, J=5.0 Hz, 2H). ¹³C NMR (101 MHz, Acetone-d₆) δ 167.0, 166.0, 164.6 (d, J=241.4 Hz), 150.9 (d, J=11.1 Hz), 143.1, 140.8, 138.1, 134.1 (d, J=9.0 Hz), 131.6 (q, J=32.4 Hz), 127.5 (2C), 126.9 (q, J=3.9 Hz, 2C), 125.1 (q, J=271.3 Hz), 111.3, 105.1 (d, J=24.1 Hz), 104.0 (d, J=25.8 Hz), 40.7. MSESI m/z: 395.0491 (C₁₈H₁₁F₄N₂O₂S⁻ requires 395.0477).

### Example 25. Preparation of 3-fluoro-5-(((2-(4-(trifluoromethyl)phenyl)thiazol-4-yl)methyl)amino)benzoic Acid (8d)

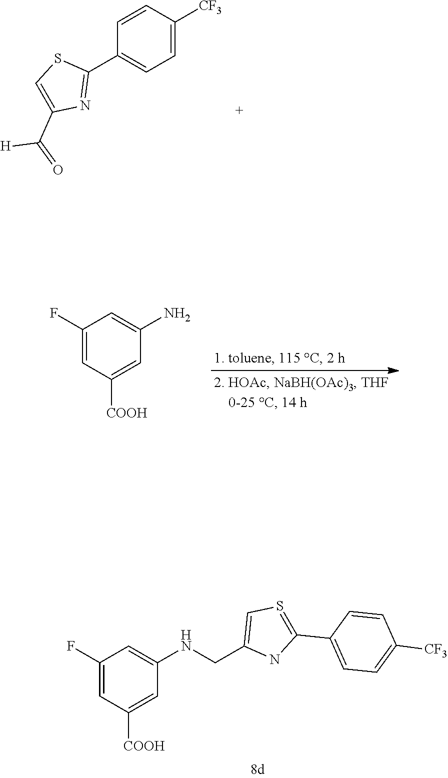

A mixture of 2-(4-(trifluoromethyl)phenyl)thiazole-4-carbaldehyde (26 mg, 0.1 mmol) and 3-amino-5-fluorobenzoic acid (16 mg, 0.1 mmol) in toluene (1 mL) was stirred at 115° C. for 2 h. The solvent was removed under reduced pressure by rotary evaporation. The residue was dissolved in THF (1 mL) and cooled to 0° C. Sodium triacetoxyborohydride (42 mg, 0.2 mmol) was added and the mixture was stirred at 0° C. for 15 min. Acetic acid (1 μL) was added and the mixture was then warmed to room temperature and stirred. After the reaction was judged to be completed by TLC (14 h), it was quenched with water and extracted with EtOAc. The organic layer was washed with water and brine and concentrated under reduced pressure by rotary evaporation. The crude residue was purified by flash column chromatography (SiO₂: 0-10% MeOH in DCM with 1% formic acid) to afford compound 8d as a yellow amorphous solid (23 mg, 58%). ¹H NMR (400 MHz, DMSO-d₆) δ 13.02 (s, 1H), 8.15 (d, J=8.1 Hz, 2H), 7.86 (d, J=8.1 Hz, 2H), 7.62 (s, 1H), 7.15 (t, J=1.8 Hz, 1H), 6.92 (t, J=5.8 Hz, 1H), 6.86-6.74 (m, 1H), 6.69-6.63 (m, 1H), 4.49 (d, J=5.8 Hz, 2H). ¹³C NMR (101 MHz, DMSO-d₆) δ 166.8, 165.3, 163.1 (d, J=240.1 Hz), 156.0, 150.4 (d, J=11.1 Hz), 136.5, 133.5 (d, J=9.0 Hz), 129.9 (q, J=32.0 Hz), 126.8 (2C), 126.2 (q, J=3.8 Hz, 2C), 124.0 (q, J=272.2 Hz), 117.6, 110.0, 102.6 (d, J=23.5 Hz), 102.0 (d, J=25.3 Hz), 43.1. MSESI m/z: 395.0474 (C₁₈H₁₁F₄N₂O₂S⁻ requires 395.0477).

### Example 26. Preparation of 3-fluoro-5-(((2-(4-fluorophenyl)thiazol-4-yl)methyl)amino)benzoic Acid (8e)

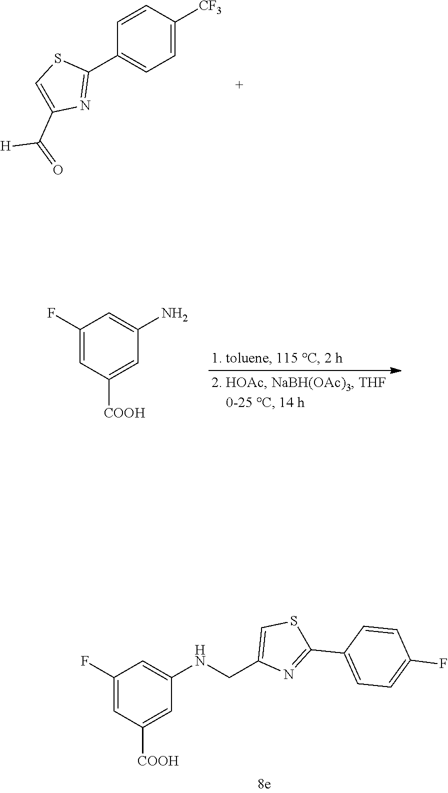 [SMILES: O=Cc1csc(-c2ccc(C(F)(F)F)cc2)n1 ; Nc1cc(F)cc(C(=O)O)c1 ; O=C(O)c1cc(F)cc(NCc2csc(-c3ccc(F)cc3)n2)c1]

A mixture of 2-(4-fluorophenyl)thiazole-4-carbaldehyde (21 mg, 0.1 mmol) and 3-amino-5-fluorobenzoic acid (16 mg, 0.1 mmol) in toluene (1 mL) was stirred at 115° C. for 2 h. The solvent was removed under reduced pressure by rotary evaporation. The residue was dissolved in THF (1 mL) and cooled to 0° C. Sodium triacetoxyborohydride (42 mg, 0.2 mmol) was added and the mixture was stirred at 0° C. for 15 min. Acetic acid (1 μL) was added and the mixture was then warmed to room temperature and stirred. After the reaction was judged to be completed by TLC (14 h), it was quenched with water and extracted with EtOAc. The organic layer was washed with water and brine and concentrated under reduced pressure by rotary evaporation. The crude residue was purified by flash column chromatography (SiO₂: 0-10% MeOH in DCM with 1% formic acid) followed by preparative TLC (SiO₂: 15% MeOH in DCM) to afford compound 8e as a yellow amorphous solid (18 mg, 52%). ¹H NMR (400 MHz, Acetone-d₆) δ 8.09-8.00 (m, 2H), 7.43 (s, 1H), 7.35-7.19 (m, 3H), 6.94 (ddd, J=9.3, 2.5, 1.4 Hz, 1H), 6.72 (dt, J=11.6, 2.3 Hz, 1H), 6.19 (s, 1H), 4.57 (s, 2H). ¹³C NMR (101 MHz, Acetone-d₆) δ 167.4, 167.1 (d, J=3.5 Hz), 164.7 (d, J=248.3 Hz), 164.6 (d, J=241.0 Hz), 156.6, 151.5 (d, J=11.1 Hz), 133.9 (d, J=9.9 Hz), 131.2 (d, J=3.3 Hz), 129.3 (d, J=8.7 Hz, 2C), 116.9 (d, J=22.2 Hz, 2C), 116.1, 111.2 (d, J=2.1 Hz), 104.4 (d, J=24.0 Hz), 103.7 (d, J=25.9 Hz), 44.6. MSESI m/z: 345.0510 (C₁₇H₁₁F₂N₂O₂S⁻ requires 345.0509).

### Example 27. Preparation of 3-(((1-(4-(trifluoromethyl)phenyl)-1H-1,2,3-triazol-4-yl)methyl)amino)benzoic Acid (12a)

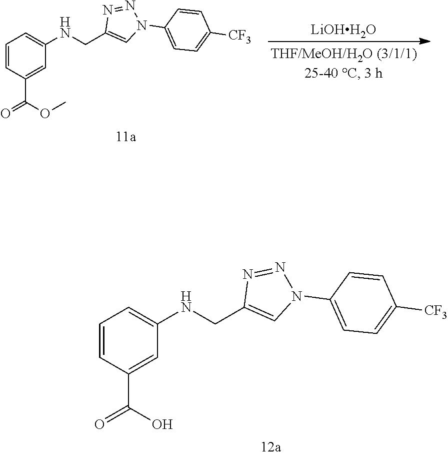 [SMILES: COC(=O)c1cccc(NCc2cn(-c3ccc(C(F)(F)F)cc3)nn2)c1 ; "LiOH·H2O" ; O=C(O)c1cccc(NCc2cn(-c3ccc(C(F)(F)F)cc3)nn2)c1]

A mixture of compound 11a (20 mg, 53 μmol) and lithium hydroxide monohydrate (22 mg, 0.53 mmol) in THF/MeOH/H₂O (v/v/v=3:1:1, 1 mL) was stirred at 25° C. for 2 h and heated to 40° C. After the reaction was judged to be completed by TLC (1 hours), it was diluted with water, acidified aqueous 1N HCl, and extracted with EtOAc. The organic layer was washed with water and brine and concentrated under reduced pressure by rotary evaporation. The crude residue was purified by flash column chromatography (SiO₂, 0-20% MeOH/DCM with 1% AcOH) to give compound 12a as a yellow amorphous solid (15 mg, 78%). ¹H NMR (400 MHz, DMSO-d₆) δ 12.69 (s, 1H), 8.86 (s, 1H), 8.16 (d, J=8.4 Hz, 2H), 7.97 (d, J=8.4 Hz, 2H), 7.28-7.25 (m, 1H), 7.23-7.12 (m, 2H), 6.93-6.87 (m, 1H), 6.47 (t, J=5.7 Hz, 1H), 4.43 (d, J=5.7 Hz, 2H). ¹³C NMR (101 MHz, DMSO-d₆) δ 167.8, 148.4, 147.1, 139.4, 131.5, 129.0, 128.5 (q, J=32.2 Hz), 127.2 (q, J=4.0 Hz, 2C), 123.8 (q, J=272.2 Hz), 121.4, 120.4 (2C), 117.2, 116.2, 113.2, 38.5. MSESI m/z: 361.0912 (C₁₇H₁₂F₃N₄O₂⁻ requires 361.0912).

The intermediate compound 11a was prepared as follows.

### a. Preparation of Methyl 3-(prop-2-yn-1-ylamino)benzoate (9a)

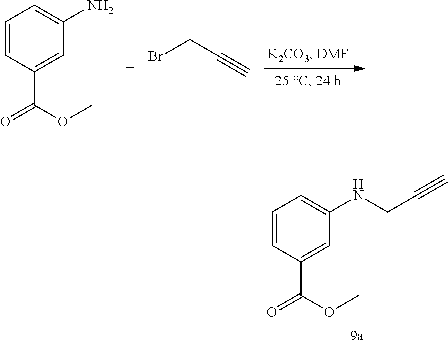

A mixture of methyl 3-aminobenzoate (151 mg, 1 mmol) and potassium carbonate (69 mg, 0.5 mmol) in DMF (1.25 mL) was stirred at 25° C. for 5 min. A solution of propargyl bromide (80% in toluene, 22.3 μL, 0.25 mmol) in DMF (0.25 mL) was added to the reaction mixture dropwise with a syringe. The resulting mixture was stirred at 25° C. for 24 hours. The reaction was quenched with water and extracted with EtOAc. The organic layer was washed with water and brine and concentrated under reduced pressure by rotary evaporation. The crude residue was purified by flash column chromatography (SiO₂, 0-30% EtOAc/hexanes) to give compound 9a as a yellow oil (26 mg, 55%). ¹H NMR (400 MHz, CDCl₃) δ 7.47 (dt, J=7.7, 1.3 Hz, 1H), 7.37 (dd, J=2.6, 1.5 Hz, 1H), 7.32-7.23 (m, 1H), 6.90 (ddd, J=8.1, 2.6, 1.0 Hz, 1H), 3.99 (d, J=2.5 Hz, 2H), 3.90 (s, 3H), 2.24 (t, J=2.4 Hz, 1H).

### b. Preparation of Methyl 3-(((1-(4-(Trifluoromethyl)phenyl)-1H-1,2,3-triazol-4-yl)methyl)amino)benzoate (11a)

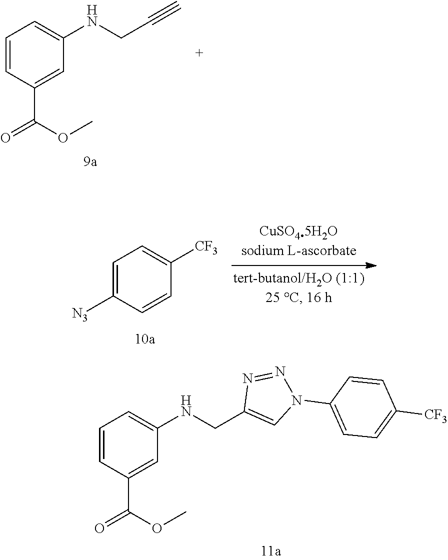 [SMILES: C#CCNc1cccc(C(=O)OC)c1 ; [N-]=[N+]=Nc1ccc(C(F)(F)F)cc1 ; COC(=O)c1cccc(NCc2cn(-c3ccc(C(F)(F)F)cc3)nn2)c1]

A mixture of compound 9a (14 mg, 74 μmol), 1-azido-4-(trifluoromethyl)benzene 10a (0.5 M in tert-butyl methyl ether, 0.22 mL, 0.11 mmol), copper (II) sulfate pentahydrate (1.8 mg, 7.4 μmol), and sodium L-ascorbate (7.3 mg, 37 μmol) in tert-butanol/H₂O (v/v=1:1, 1 mL) was stirred at 25° C. After the reaction was judged to be completed by TLC (16 hours), the reaction mixture was concentrated under reduced pressure by rotary evaporation. The crude residue was purified by flash column chromatography (SiO₂, 0-50% EtOAc/hexanes) to give compound 11a as a yellow amorphous solid (20 mg, 72%). ¹H NMR (400 MHz, CDCl₃) δ 7.96 (s, 1H), 7.86 (d, J=8.5 Hz, 2H), 7.78 (d, J=8.5 Hz, 2H), 7.47-7.41 (m, 1H), 7.38 (t, J=2.0 Hz, 1H), 7.28-7.19 (m, 2H), 6.88 (dd, J=8.4, 2.6 Hz, 1H), 4.60 (s, 2H), 3.89 (s, 3H).

### Example 28. Preparation of 3-(((1-(4-fluorophenyl)-1H-1,2,3-triazol-4-yl)methyl)amino)benzoic Acid (12b)

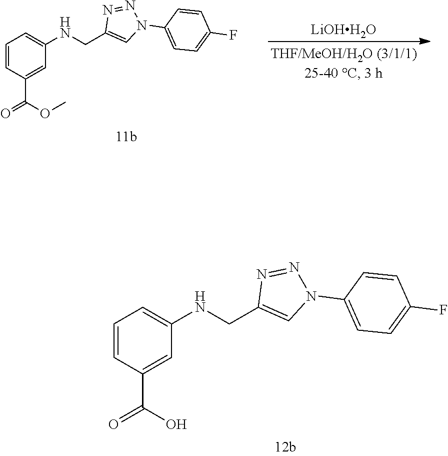 [SMILES: COC(=O)c1cccc(NCc2cn(-c3ccc(F)cc3)nn2)c1 ; "LiOH·H2O" ; O=C(O)c1cccc(NCc2cn(-c3ccc(F)cc3)nn2)c1]

A mixture of compound 11b (20 mg, 61 μmol) and lithium hydroxide monohydrate (26 mg, 0.61 mmol) in THF/MeOH/H₂O (v/v/v=3:1:1, 1 mL) was stirred at 25° C. for 2 h and heated to 40° C. After the reaction was judged to be completed by TLC (1 hours), it was diluted with water, acidified aqueous 1N HCl, and extracted with EtOAc. The organic layer was washed with water and brine and concentrated under reduced pressure by rotary evaporation. The crude residue was purified by flash column chromatography (SiO₂, 0-20% MeOH/DCM with 1% AcOH) to give compound 12b as a yellow amorphous solid (15 mg, 78%). ¹H NMR (400 MHz, DMSO-d₆) δ 12.71 (s, 1H), 8.68 (s, 1H), 7.93 (dd, J=9.1, 4.7 Hz, 1H), 7.44 (t, J=9.1 Hz, 2H), 7.26 (t, J=1.9 Hz, 1H), 7.23-7.14 (m, 2H), 6.92-6.86 (m, 1H), 6.43 (t, J=5.7 Hz, 1H), 4.41 (d, J=5.7 Hz, 2H). ¹³C NMR (101 MHz, DMSO-d₆) δ 167.8, 161.5 (d, J=245.4 Hz), 148.4, 146.7, 133.2 (d, J=2.9 Hz), 131.5, 129.0, 122.2 (d, J=8.8 Hz, 2C), 121.3, 117.1, 116.7 (d, J=23.3 Hz, 2C), 116.2, 113.2, 38.5. MSESI m/z: 311.0945 (C₁₆H₁₂FN₄O₂⁻ requires 311.0944).

The intermediate compound 11b was prepared as follows.

### a. Preparation of methyl 3-Fluoro-5-(prop-2-yn-1-ylamino)benzoate (9b)

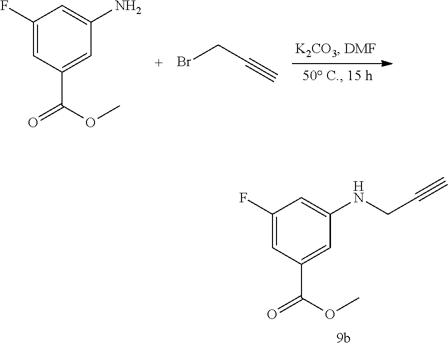

A mixture of methyl 3-amino-5-fluorobenzoate (169 mg, 1 mmol) and potassium carbonate (83 mg, 0.6 mmol) in DMF (1.25 mL) was stirred at 25° C. for 5 min. A solution of propargyl bromide (80% in toluene, 44.5 μL, 0.5 mmol) in DMF (0.25 mL) was added to the reaction mixture dropwise with a syringe. The resulting mixture was stirred at 50° C. for 15 hours. The reaction was cooled to room temperature, quenched with water, and extracted with EtOAc. The organic layer was washed with water and brine and concentrated under reduced pressure by rotary evaporation. The crude residue was purified by flash column chromatography (SiO₂, 0-30% EtOAc/hexanes) to give compound 9b as a yellow amorphous solid (46 mg, 44%). ¹H NMR (400 MHz, CDCl₃) δ 7.16 (dd, J=2.3, 1.4 Hz, 1H), 7.13 (ddd, J=9.0, 2.3, 1.4 Hz, 1H), 6.64-6.52 (m, 1H), 3.96 (d, J=2.4 Hz, 2H), 3.90 (s, 3H), 2.26 (t, J=2.4 Hz, 1H).

### b. Preparation of Methyl 3-(((1-(4-Fluorophenyl)-1H-1,2,3-triazol-4-yl)methyl)amino)benzoate (11b)

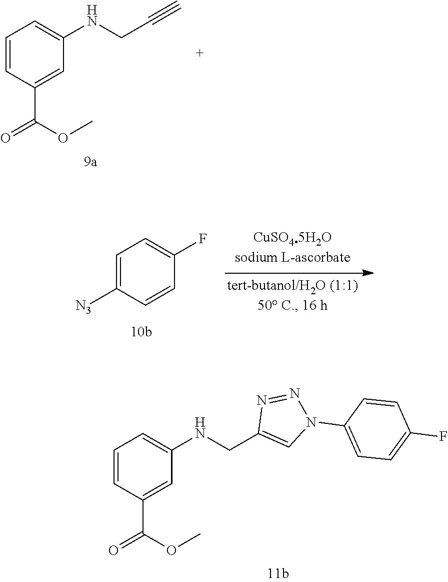 [SMILES: C#CCNc1cccc(C(=O)OC)c1 ; [N-]=[N+]=Nc1ccc(F)cc1 ; COC(=O)c1cccc(NCc2cn(-c3ccc(F)cc3)nn2)c1]

A mixture of compound 9a (14 mg, 74 μmol), 1-azido-4-fluorobenzene 10b (0.5 M in tert butyl methyl ether, 0.22 mL, 0.11 mmol), copper (II) sulfate pentahydrate (1.8 mg, 7.4 μmol), and sodium L-ascorbate (7.3 mg, 37 μmol) in tert-butanol/H₂O (v/v=1:1, 2 mL) was stirred at 25° C. After the reaction was judged to be completed by TLC (16 hours), the reaction mixture was concentrated under reduced pressure by rotary evaporation. The crude residue was purified by flash column chromatography (SiO₂, 0-50% EtOAc/hexanes) to give compound 11b as a yellow amorphous solid (23 mg, 95%). ¹H NMR (400 MHz, CDCl₃) δ 7.85 (s, 1H), 7.74-7.58 (m, 2H), 7.42 (d, J=7.7 Hz, 1H), 7.37 (t, J=2.0 Hz, 1H), 7.25-7.14 (m, 3H), 6.87 (dd, J=8.2, 2.6 Hz, 1H), 4.57 (s, 2H), 3.89 (s, 3H).

### Example 29. Preparation of 3-fluoro-5-(((1-(4-(trifluoromethyl)phenyl)-1H-1,2,3-triazol-4-yl)methyl)amino)benzoic Acid (12c)

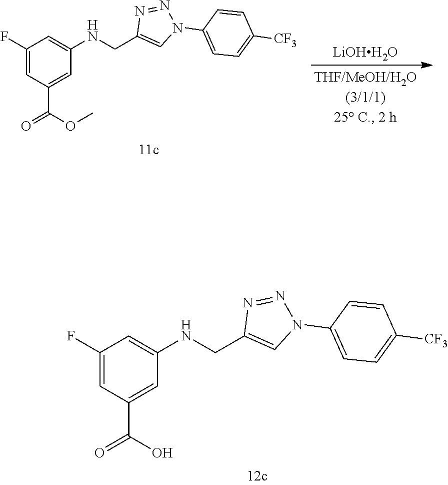 [SMILES: COC(=O)c1cc(F)cc(NCc2cn(-c3ccc(C(F)(F)F)cc3)nn2)c1 ; "LiOH·H2O" ; O=C(O)c1cc(F)cc(NCc2cn(-c3ccc(C(F)(F)F)cc3)nn2)c1]

A mixture of compound 11c (36 mg, 91 μmol) and lithium hydroxide monohydrate (38 mg, 0.91 mmol) in THF/MeOH/H₂O (v/v/v=3:1:1, 2 mL) was stirred at 25° C. After the reaction was judged to be completed by TLC (2 hours), it was diluted with water, acidified aqueous HCl (1M), and extracted with EtOAc. The organic layer was washed with water and brine and concentrated under reduced pressure by rotary evaporation. The crude residue was purified by flash column chromatography (SiO₂, 0-20% MeOH/DCM with 1% AcOH) to give compound 12c as a yellow amorphous solid (30 mg, 86%). ¹H NMR (400 MHz, Acetone-d₆) δ 8.67 (s, 1H), 8.16 (d, J=8.4 Hz, 2H), 7.95 (d, J=8.4 Hz, 2H), 7.41-7.21 (m, 1H), 6.94 (ddd, J=9.3, 2.4, 1.3 Hz, 1H), 6.75 (dt, J=11.5, 2.4 Hz, 1H), 6.12 (s, 1H), 4.60 (s, 2H). ¹³C NMR (101 MHz, Acetone-d₆) δ 167.1, 164.6 (d, J=241.0 Hz), 151.2 (d, J=11.2 Hz), 147.8, 140.9, 134.1, 130.4 (q, J=32.8 Hz), 128.0 (q, J=3.8 Hz, 2C), 124.9 (q, J=271.3 Hz), 121.6, 121.3 (2C), 111.3, 104.6 (d, J=23.8 Hz), 103.6 (d, J=26.0 Hz), 39.7. MSESI m/z: 379.0818 (C₁₇H₁₁F₄N₄O₂⁻ requires 379.0818).

The intermediate compound 11c was prepared as follows.

### a. Preparation of methyl 3-Fluoro-5-(((1-(4-(trifluoromethyl)phenyl)-11H-1,2,3-triazol-4-yl)methyl)amino)benzoate (11c)

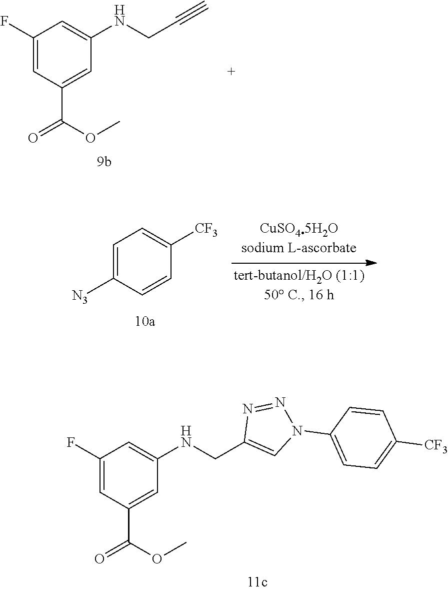 [SMILES: C#CCNc1cc(F)cc(C(=O)OC)c1 ; [N-]=[N+]=Nc1ccc(C(F)(F)F)cc1 ; COC(=O)c1cc(F)cc(NCc2cn(-c3ccc(C(F)(F)F)cc3)nn2)c1]

A mixture of compound 9b (21 mg, 0.1 mmol), 1-azido-4-(trifluoromethyl)benzene 10a (0.5 M in tert-butyl methyl ether, 0.3 mL, 0.15 mmol), copper (II) sulfate pentahydrate (2.5 mg, 10 μmol), and sodium L-ascorbate (10 mg, 50 μmol) in tert-butanol/H₂O (v/v=1:1, 2 mL) was stirred at 50° C. After the reaction was judged to be completed by TLC (16 hours), the reaction mixture was concentrated under reduced pressure by rotary evaporation. The crude residue was purified by flash column chromatography (SiO₂, 0-50% EtOAc/hexanes) to give compound 11c as a yellow amorphous solid (36 mg, 90%). ¹H NMR (400 MHz, CDCl₃) δ 7.98 (s, 1H), 7.87 (d, J=8.5 Hz, 2H), 7.79 (d, J=8.5 Hz, 2H), 7.19 (dd, J=2.3, 1.4 Hz, 1H), 7.09 (ddd, J=9.0, 2.3, 1.3 Hz, 1H), 6.66-6.55 (m, 1H), 4.58 (s, 2H), 3.89 (s, 3H).

### Example 30. Preparation of 3-fluoro-5-(((1-(4-fluorophenyl)-1H-1,2,3-triazol-4-yl)methyl)amino)benzoic Acid (12d)

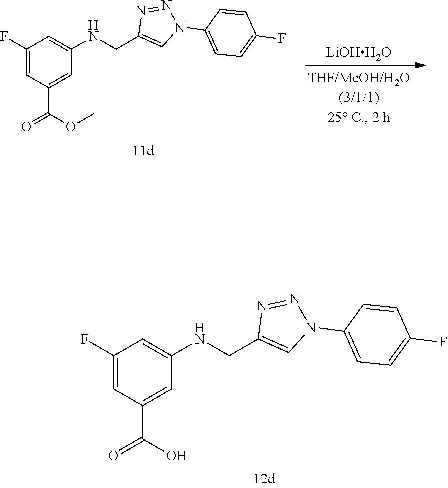

A mixture of compound 11d (25 mg, 73 μmol) and lithium hydroxide monohydrate (30 mg, 0.73 mmol) in THF/MeOH/H₂O (v/v/v=3:1:1, 2 mL) was stirred at 25° C. After the reaction was judged to be completed by TLC (2 hours), it was diluted with water, acidified aqueous 1N HCl, and extracted with EtOAc. The organic layer was washed with water and brine and concentrated under reduced pressure by rotary evaporation. The crude residue was purified by flash column chromatography (SiO₂, 0-20% MeOH/DCM with 1% AcOH) to give compound 12d as a yellow amorphous solid (23 mg, 96%). ¹H NMR (400 MHz, Acetone-d₆) δ 8.49 (s, 1H), 8.18-7.71 (m, 2H), 7.36 (t, J=8.7 Hz, 1H), 7.30-7.19 (m, 1H), 6.94 (ddd, J=9.3, 2.4, 1.3 Hz, 1H), 6.75 (dt, J=11.5, 2.4 Hz, 1H), 6.08 (s, 1H), 4.57 (s, 2H). ¹³C NMR (101 MHz, Acetone-d₆) δ 167.1, 164.6 (d, J=241.3 Hz), 163.1 (d, J=246.2 Hz), 151.3 (d, J=11.2 Hz), 147.4, 134.7 (d, J=3.0 Hz), 134.1, 123.3 (d, J=8.8 Hz, 2C), 121.6, 117.4 (d, J=23.5 Hz, 2C), 111.3, 104.5 (d, J=23.8 Hz), 103.6 (d, J=26.0 Hz), 39.7. MSESI m/z: 329.0851 (C₁₆H₁₁F₂N₄O₂⁻ requires 329.0850).

The intermediate compound 11d was prepared as follows.

### a. Preparation of methyl 3-Fluoro-5-(((1-(4-fluorophenyl)-1H-1,2,3-triazol-4-yl)methyl)amino)benzoate (11d)

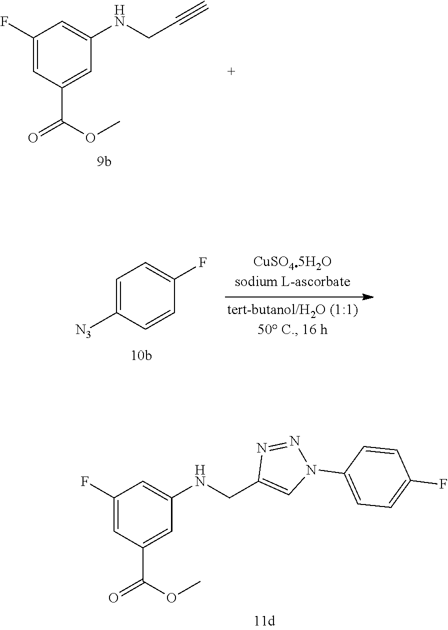 [SMILES: C#CCNc1cc(F)cc(C(=O)OC)c1 ; [N-]=[N+]=Nc1ccc(F)cc1 ; COC(=O)c1cc(F)cc(NCc2cn(-c3ccc(F)cc3)nn2)c1]

A mixture of compound 9b (21 mg, 0.1 mmol), 1-azido-4-fluorobenzene 10b (0.5 M in tert-butyl methyl ether, 0.3 mL, 0.15 mmol), copper (II) sulfate pentahydrate (2.5 mg, 10 μmol), and sodium L-ascorbate (10 mg, 50 μmol) in tert-butanol/H₂O (v/v=1:1, 2 mL) was stirred at 50° C. After the reaction was judged to be completed by TLC (16 hours), the reaction mixture was concentrated under reduced pressure by rotary evaporation. The crude residue was purified by flash column chromatography (SiO₂, 0-50% EtOAc/hexanes) to give compound 11d as a yellow amorphous solid (25 mg, 72%). ¹H NMR (400 MHz, CDCl₃) δ 7.87 (s, 1H), 7.75-7.63 (m, 2H), 7.24-7.17 (m, 3H), 7.08 (ddd, J=9.0, 2.3, 1.3 Hz, 1H), 6.67-6.43 (m, 1H), 4.55 (s, 2H), 3.89 (s, 3H).

### Example 31. Preparation of 5-(((4′-fluoro-[1,1′-biphenyl]-4-yl)methyl)amino)-2-hydroxybenzoic acid

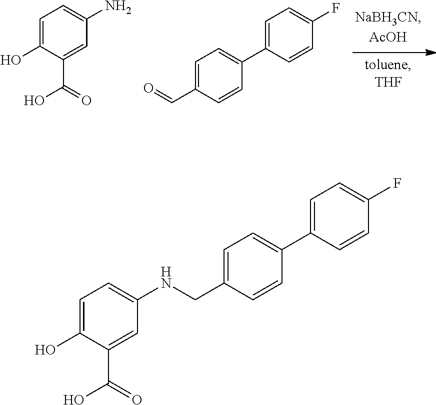 [SMILES: Nc1ccc(O)c(C(=O)O)c1 ; O=Cc1ccc(-c2ccc(F)cc2)cc1 ; O=C(O)c1cc(NCc2ccc(-c3ccc(F)cc3)cc2)ccc1O]

5-Amino-2-hydroxybenzoic acid (50 mg, 0.33 mmol) and 4′-fluoro-[1,1′-biphenyl]-4-carbaldehyde (65 mg, 0.33 mmol) were added to 3 mL toluene and refluxed for 2 hours. The toluene was removed by boiling, then the reaction was cooled to 0° C. followed by the addition of 3 mL of THF and sodium cyanoborohydride (41 mg, 0.65 mmol). The reaction mixture was stirred for 15 minutes and 3 μL of glacial acetic acid was added. The reaction mixture was stirred for 16 hours at room temperature. The reaction was quenched with water and extracted with ethyl acetate (3×30 mL). The combined organic fractions were dried with sodium sulfate and purified by recrystallization. A yellow amorphous solid 5-(((4′-fluoro-[1,1′-biphenyl]-4-yl)methyl)amino)-2-hydroxybenzoic acid was obtained (77 mg, 70% yield. ¹H NMR (400 MHz, DMSO-d₆) δ 10.51 (s, 1H), 7.69 (dd, J=8.8, 5.5 Hz, 2H), 7.65-7.57 (m, 2H), 7.44 (d, J=8.1 Hz, 2H), 7.34-7.20 (m, 2H), 7.00 (d, J=2.9 Hz, 1H), 6.91 (dd, J=8.8, 3.0 Hz, 1H), 6.74 (d, J=8.8 Hz, 1H), 4.27 (s, 2H). ¹³C NMR (151 MHz, DMSO-d₆) δ 172.5, 163.0, 161.4, 153.4, 139.7, 138.0, 136.9 (d, J=3.1 Hz), 129.0 (d, J=8.2 Hz) (2C), 128.4 (2C), 128.1 (d, J=299.8 Hz), 127.0 (2C), 122.5, 117.9, 116.1 (d, J=21.3 Hz) (2C), 113.0, 47.4. HRMS (ESI, m/z) for C₂₀H₁₆FNO₃ [M−H]: expected, 336.1041; found, 336.1055; 4.1 ppm.

### Examples 32-48

General Procedure 3: Reductive amination. A mixture of aldehyde (1 eq) and aniline (1 eq) in toluene (5 mL) was stirred at 110° C. for 1.5 hr. The solvent was boiled off under a stream of nitrogen gas. The residue was dissolved in THF (0.05 M), and sodium triacetoxyborohydride (2 eq) or sodium cyanoborohydride (2 eq) were added and the reaction mixture was stirred at room temperature for 15 min. Acetic acid (0.05 eq) was added and the mixture was stirred for 16 hr. The reaction was quenched with deionized water and extracted with EtOAc. The organic phase was washed with deionized water and brine, dried over sodium sulfate, filtered, and concentrated under reduced pressure by rotary evaporation. The crude residue was purified by flash column chromatography (SiO₂, eluted with a gradient of 0-20% MeOH in DCM).

General Procedure 4: Suzuki to access functionalized aldehydes. A mixture of aldehyde (1 eq), boronic acid (1 eq), K₂CO₃ (3 eq), and Pd(dppf)Cl₂·CH₂Cl₂ (0.1 eq) in dioxane/H₂O (v/v=9:1, 0.1 M) was stirred at 90° C. under a nitrogen atmosphere for 3 hr. The reaction was quenched with deionized water and extracted with EtOAc. The organic phase was washed with deionized water and brine, dried over sodium sulfate, filtered, and concentrated under reduced pressure by rotary evaporation. The crude residue was purified by flash column chromatography (SiO₂, eluted with 0-40% EtOAc in hexanes).

### Example 32. Preparation of 2-hydroxy-5-(((4′-(trifluoromethyl)-[1,1′-biphenyl]-4-yl)methyl)amino)benzoic acid

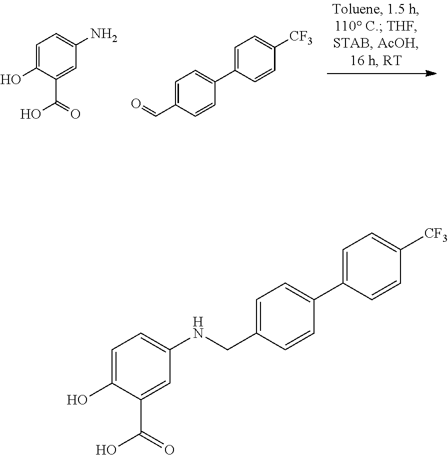 [SMILES: Nc1ccc(O)c(C(=O)O)c1 ; O=Cc1ccc(-c2ccc(C(F)(F)F)cc2)cc1 ; O=C(O)c1cc(NCc2ccc(-c3ccc(C(F)(F)F)cc3)cc2)ccc1O]

The title compound was synthesized in accordance with General Procedure 3, employing 5-amino-2-hydroxybenzoic acid (100 mg, 0.653 mmol) and 4′-(trifluoromethyl)-[1,1′-biphenyl]-4-carbaldehyde (163 mg, 0.653 mmol). Sodium triacetoxyborohydride (277 mg, 1.31 mmol) was employed as the reducing agent. The product was obtained in 14% yield (36 mg) as a white amorphous solid. ¹H NMR (400 MHz, DMSO-d₆) δ 7.89 (d, J=8.2 Hz, 2H), 7.80 (d, J=8.2 Hz, 2H), 7.71 (d, J=8.2 Hz, 2H), 7.49 (d, J=8.0 Hz, 2H), 6.98 (d, J=2.9 Hz, 1H), 6.90 (dd, J=8.8, 3.0 Hz, 1H), 6.74 (s, 1H), 4.29 (s, 2H). ¹³C NMR (214 MHz, DMSO-d₆) δ 172.5, 144.5, 141.2, 137.4, 128.4, 128.1 (d, J=31.6 Hz) (2C), 127.9, 127.8 (2C), 127.5 (2C), 126.2 (d, J=3.8 Hz) (2C), 125.5, 124.2, 122.2, 117.9, 79.4 (dd, J=34.2, 32.1 Hz), 47.3-47.2 (m), 40.5. HRMS (ESI, m/z) for C₂₁H₁₆F₃NO₃ [M−H]: expected, 386.1010; found, 386.1013; 0.7 ppm. Example 33. Preparation of 5-((4′-Fluoro-[1,1′-biphenyl]-4-yl)methoxy)-2-hydroxybenzoic acid.

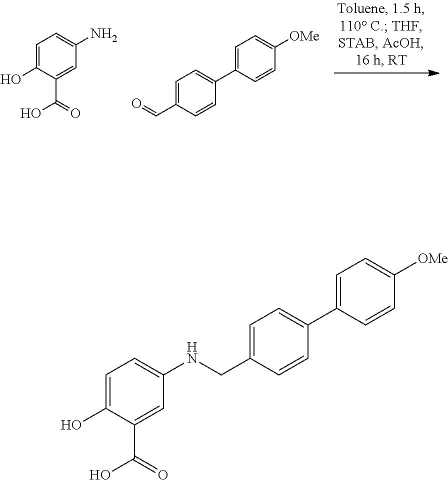 [SMILES: Nc1ccc(O)c(C(=O)O)c1 ; COc1ccc(-c2ccc(C=O)cc2)cc1 ; COc1ccc(-c2ccc(CNc3ccc(O)c(C(=O)O)c3)cc2)cc1]

The title compound was synthesized in accordance with General Procedure 3, employing 4′-methoxy-[1,1′-biphenyl]-4-carbaldehyde (70 mg, 0.33 mmol) and 5-amino-2-hydroxybenzoic acid (51 mg, 0.33 mmol). Sodium triacetoxyborohydride (140 mg, 0.66 mmol) was employed as the reducing agent. The product was obtained in 9.5% yield (11 mg) as a white amorphous solid. ¹H NMR (400 MHz, DMSO-d₆) δ 10.59 (s, 1H), 7.58 (t, J=8.5 Hz, 4H), 7.41 (d, J=8.2 Hz, 2H), 7.07-6.95 (m, 3H), 6.90 (dd, J=8.8, 3.0 Hz, 1H), 6.73 (d, J=8.9 Hz, 1H), 4.24 (s, 2H), 3.79 (s, 3H). ¹³C NMR (151 MHz, DMSO-d₆) δ 172.5, 159.2, 153.2, 141.8, 139.1, 138.7, 132.8, 128.3 (2C), 128.1 (2C), 126.5 (2C), 122.3, 117.9, 114.8 (2C), 113.0, 111.9, 55.6, 47.4. HRMS (ESI, m/z) for C₂₁H₁₉NO₄ [M−H]: expected, 348.1241; found, 348.1230; 3.2 ppm.

### Example 34. Preparation of 2-Hydroxy-5-(((4′-(methylsulfonyl)-[1,1′-biphenyl]-4-yl)methyl)amino)benzoic acid.)

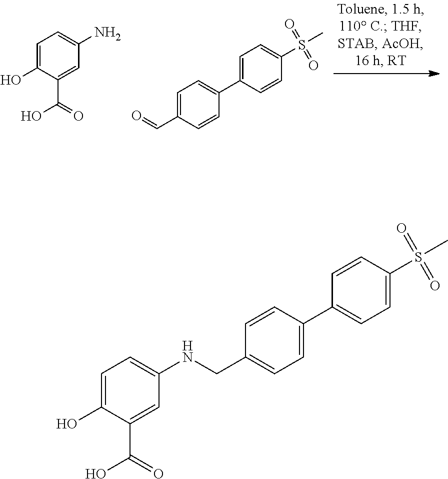 [SMILES: Nc1ccc(O)c(C(=O)O)c1 ; CS(=O)(=O)c1ccc(-c2ccc(C=O)cc2)cc1 ; CS(=O)(=O)c1ccc(-c2ccc(CNc3ccc(O)c(C(=O)O)c3)cc2)cc1]

The title compound was synthesized in accordance with General Procedure 3, employing 4′-(methylsulfonyl)-[1,1′-biphenyl]-4-carbaldehyde (82 mg, 0.32 mmol) and 5-amino-2-hydroxybenzoic acid (48 mg, 0.32 mmol). Sodium triacetoxyborohydride (130 mg, 0.63 mmol) was employed as the reducing agent. The product was obtained in 2.2% yield (2.8 mg) as a white amorphous solid. ¹H NMR (400 MHz, DMSO-d₆) δ 8.05-7.90 (m, 4H), 7.80-7.66 (m, 2H), 7.50 (d, J=8.2 Hz, 2H), 6.89 (d, J=2.9 Hz, 1H), 6.81 (dd, J=8.8, 3.0 Hz, 1H), 6.59 (d, J=8.7 Hz, 1H), 5.94 (t, J=6.2 Hz, 1H), 4.30 (d, J=6.2 Hz, 2H), 3.26 (s, 3H). ¹³C NMR (151 MHz, DMSO) δ 164.9, 151.4, 145.5, 141.8, 141.5, 139.8, 137.1, 128.4 (2C), 128.0 (2C), 127.9 (2C), 127.6 (2C), 121.4, 118.7, 115.8, 111.0, 47.4, 44.1. HRMS (ESI, m/z) for C₂₁H₁₉NO₅S [M−H]: expected, 396.0911; found, 396.0910; 0.3 ppm.

The intermediate aldehyde was prepared as follows.

### a. 4′-(Methylsulfonyl)-[1,1′-biphenyl]-4-carbaldehyde

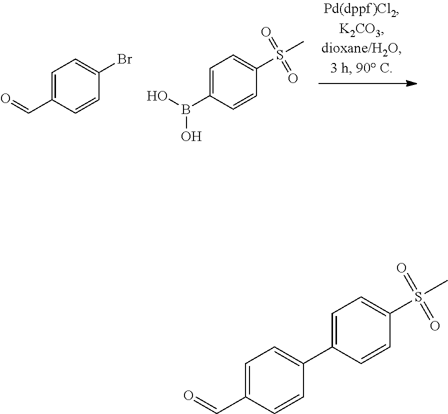 [SMILES: O=Cc1ccc(Br)cc1 ; CS(=O)(=O)c1ccc(B(O)O)cc1 ; CS(=O)(=O)c1ccc(-c2ccc(C=O)cc2)cc1]

The aldehyde was synthesized in accordance with General Procedure 4, employing 4-bromobenzaldehyde (300 mg, 1.62 mmol), (4-(methylsulfonyl)phenyl)boronic acid (324 mg, 1.62 mmol), potassium carbonate (672 mg, 4.86 mmol), and Pd(dppf)Cl₂·DCM (132 mg, 0.162 mmol). The product was obtained in 19% yield (82 mg) as a white amorphous solid. ¹H NMR (400 MHz, Acetonitrile-d₃) δ 10.09 (s, 1H), 8.03 (dd, J=20.0, 8.3 Hz, 4H), 7.80 (dd, J=17.2, 8.2 Hz, 4H), 3.11 (s, 3H).

### Example 35. Preparation of 2-Hydroxy-5-(((4′-hydroxy-[1,1′-biphenyl]-4-yl)methyl)amino)benzoic acid

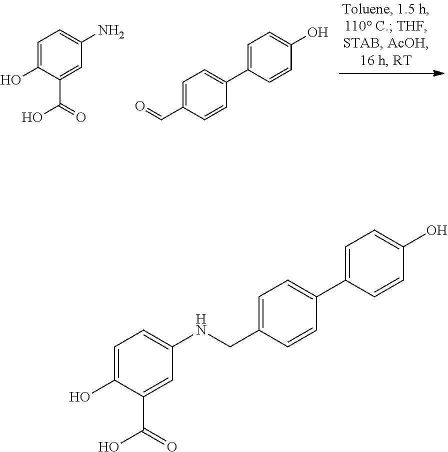 [SMILES: Nc1ccc(O)c(C(=O)O)c1 ; O=Cc1ccc(-c2ccc(O)cc2)cc1 ; O=C(O)c1cc(NCc2ccc(-c3ccc(O)cc3)cc2)ccc1O]

The title compound was synthesized in accordance with General Procedure 3, employing 4′-hydroxy-[1,1′-biphenyl]-4-carbaldehyde (80 mg, 0.4 mmol) and 5-amino-2-hydroxybenzoic acid (62 mg, 0.4 mmol). Sodium triacetoxyborohydride (170 mg, 0.81 mmol) was employed as the reducing agent. The product was obtained in 27% yield (36 mg) as a white amorphous solid. ¹H NMR (400 MHz, DMSO-d₆) δ 9.57 (s, 1H), 7.52 (d, J=8.1 Hz, 2H), 7.46 (d, J=8.6 Hz, 2H), 7.38 (d, J=8.0 Hz, 2H), 7.00 (d, J=2.9 Hz, 1H), 6.87-6.80 (m, 3H), 6.67 (d, J=8.7 Hz, 1H), 4.22 (s, 2H). ¹³C NMR (151 MHz, DMSO-d₆) δ 172.7, 157.5, 153.4, 141.4, 139.1, 138.8, 131.3, 128.2 (2C), 128.0 (2C), 126.3 (2C), 121.4, 117.5, 116.1 (2C), 114.7, 112.4, 47.5. HRMS (ESI, m/z) for C₂₀H₁₇NO₄ [M−H]: expected, 334.1085; found, 334.1091; 1.8 ppm.

### Example 36. Preparation of 5-(((4′-(tert-Butyl)-[1,1′-biphenyl]-4-yl)methyl)amino)-2-hydroxybenzoic acid

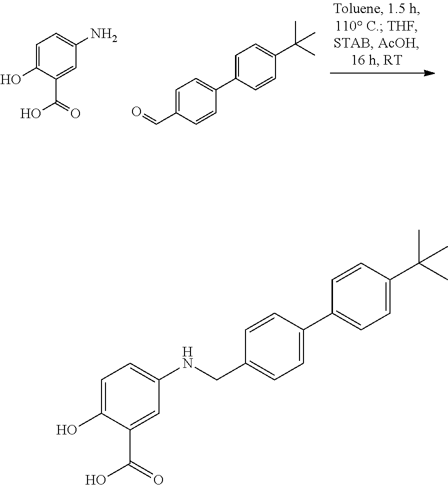 [SMILES: Nc1ccc(O)c(C(=O)O)c1 ; CC(C)(C)c1ccc(-c2ccc(C=O)cc2)cc1 ; CC(C)(C)c1ccc(-c2ccc(CNc3ccc(O)c(C(=O)O)c3)cc2)cc1]

The title compound was synthesized in accordance with General Procedure 3, employing 4′-(tert-butyl)-[1,1′-biphenyl]-4-carbaldehyde (80 mg, 0.34 mmol) and 5-amino-2-hydroxybenzoic acid (51 mg, 0.34 mmol). Sodium triacetoxyborohydride (140 mg, 0.67 mmol) was employed as the reducing agent. After flash column chromatography (SiO₂), precipitate was collected and filtered. The product was obtained in 11% yield (13.5 mg) as a white amorphous solid. ¹H NMR (400 MHz, DMSO-d₆) δ 10.55 (s, 1H), 7.59 (dd, J=10.1, 8.3 Hz, 4H), 7.45 (dd, J=12.5, 8.4 Hz, 4H), 6.99 (d, J=3.0 Hz, 1H), 6.91 (dd, J=8.9, 2.9 Hz, 1H), 6.74 (d, J=8.9 Hz, 1H), 1.31 (s, 9H). ¹³C NMR (101 MHz, DMSO-d₆) δ 172.1, 152.8, 149.6, 141.2, 139.0, 138.5, 137.1, 127.8 (2C), 126.4 (2C), 126.2 (2C), 125.6 (2C), 121.9, 117.4, 112.5, 111.3, 46.9, 34.2, 31.1 (3C). HRMS (ESI, m/z) for C₂₄H₂₅NO₃ [M−H]: expected, 374.1762; found, 374.1777; 4.0 ppm.

The intermediate aldehyde was prepared as follows.

### a. 4′-(tert-Butyl)-[1,1′-biphenyl]-4-carbaldehyde

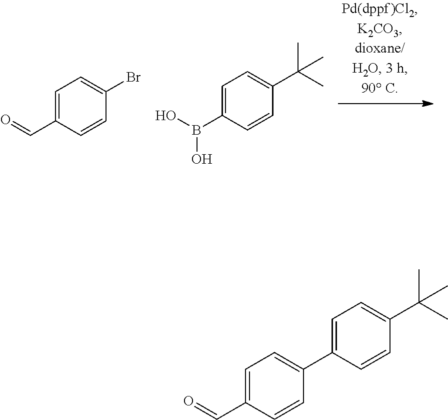 [SMILES: O=Cc1ccc(Br)cc1 ; CC(C)(C)c1ccc(B(O)O)cc1 ; CC(C)(C)c1ccc(-c2ccc(C=O)cc2)cc1]

The aldehyde was synthesized in accordance with General Procedure 4, employing 4-bromobenzaldehyde (300 mg, 1.62 mmol), (4-(tert-butyl)phenyl)boronic acid (289 mg, 1.62 mmol), potassium carbonate (672 mg, 4.86 mmol), and Pd(dppf)Cl₂·DCM (132 mg, 0.162 mmol). The product was obtained in 62% yield (242 mg) as a white amorphous solid. ¹H NMR (400 MHz, Chloroform-d) δ 10.05 (s, 1H), 7.94 (d, J=8.4 Hz, 2H), 7.76 (d, J=8.4 Hz, 2H), 7.60 (d, J=8.5 Hz, 2H), 7.51 (d, J=8.5 Hz, 2H), 1.37 (s, 9H).

### Example 37. Preparation of 5-(([1,1′-Biphenyl]-4-ylmethyl)amino)-2-hydroxybenzoic acid

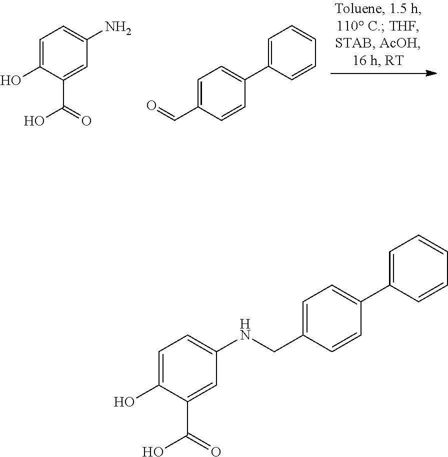 [SMILES: Nc1ccc(O)c(C(=O)O)c1 ; O=Cc1ccc(-c2ccccc2)cc1 ; O=C(O)c1cc(NCc2ccc(-c3ccccc3)cc2)ccc1O]

The title compound was synthesized in accordance with General Procedure 3, employing [1,1′-biphenyl]-4-carbaldehyde (60 mg, 0.33 mmol) and 5-amino-2-hydroxybenzoic acid (50 mg, 0.33 mmol). Sodium triacetoxyborohydride (140 mg, 0.65 mmol) was employed as the reducing agent. The product was obtained in 19% yield (20 mg) as a white amorphous solid. ¹H NMR (700 MHz, DMSO-d₆) δ 10.61 (s, 1H), 7.66 (d, J=7.5 Hz, 2H), 7.63 (d, J=8.1 Hz, 2H), 7.46 (t, J=6.9 Hz, 4H), 7.36 (t, J=7.4 Hz, 1H), 7.00 (d, J=2.8 Hz, 1H), 6.91 (dd, J=8.9, 2.9 Hz, 1H), 6.75 (d, J=8.9 Hz, 1H), 4.27 (s, 2H). ¹³C NMR (151 MHz, DMSO-d₆) δ 172.5, 153.3, 141.7, 140.5, 139.9, 139.1, 129.4 (2C), 128.3 (2C), 127.7, 127.1 (2C), 127.0 (2C), 122.3, 117.9, 113.0, 111.9, 47.4. HRMS (ESI, m/z) for C₂₀H₁₇NO₃ [M−H]: expected, 318.1135; found, 318.1136; 0.3 ppm.

### Example 38. Preparation of 5-(((4′-Fluoro-3′-nitro-[1,1′-biphenyl]-4-yl)methyl)amino)-2-hydroxybenzoic acid

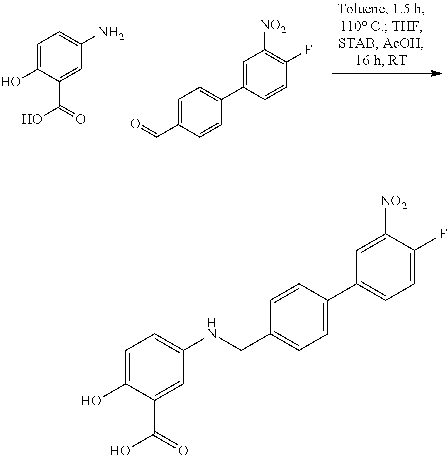 [SMILES: Nc1ccc(O)c(C(=O)O)c1 ; O=Cc1ccc(-c2ccc(F)c([N+](=O)[O-])c2)cc1 ; O=C(O)c1cc(NCc2ccc(-c3ccc(F)c([N+](=O)[O-])c3)cc2)ccc1O]

The title compound was synthesized in accordance with General Procedure 3, employing 4′-fluoro-3′-nitro-[1,1′-biphenyl]-4-carbaldehyde (80 mg, 0.33 mmol) and 5-amino-2-hydroxybenzoic acid (50 mg, 0.33 mmol). Sodium triacetoxyborohydride (140 mg, 0.65 mmol) was employed as the reducing agent. The product was obtained in 6% yield (8 mg) as a yellow amorphous solid. ¹H NMR (400 MHz, DMSO-d₆) δ 10.53 (s, 1H), 8.35 (dd, J=7.1, 2.4 Hz, 1H), 8.12 (ddd, J=8.7, 4.3, 2.4 Hz, 1H), 7.75-7.64 (m, 3H), 7.49 (d, J=8.0 Hz, 2H), 6.98 (d, J=2.9 Hz, 1H), 6.90 (dd, J=8.8, 3.0 Hz, 1H), 6.74 (d, J=8.8 Hz, 1H), 4.29 (s, 2H). ¹³C NMR (151 MHz, DMSO-d₆) δ 172.5, 155.2, 153.4 (d, J=22.1 Hz), 141.6, 141.2, 137.8 (d, J=8.1 Hz), 137.6 (d, J=3.8 Hz), 135.7, 134.5 (d, J=8.7 Hz), 128.5 (2C), 127.3 (2C), 124.0 (d, J=2.2 Hz), 122.4, 119.4 (d, J=20.7 Hz), 117.9, 113.0, 111.9, 47.2. HRMS (ESI, m/z) for C₂₀H₁₅FN₂O₅ [M−H]: expected, 381.0892; found, 381.0886; −1.6 ppm.

The intermediate aldehyde was prepared as follows.

### a. 4′-Fluoro-3′-nitro-[1,1′-biphenyl]-4-carbaldehyde

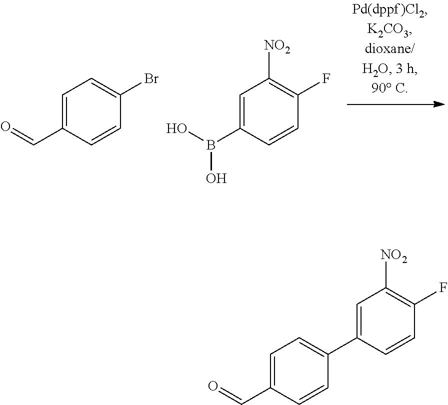 [SMILES: O=Cc1ccc(Br)cc1 ; O=[N+]([O-])c1cc(B(O)O)ccc1F ; O=Cc1ccc(-c2ccc(F)c([N+](=O)[O-])c2)cc1]

The aldehyde was synthesized in accordance with General Procedure 4, employing 4-bromobenzaldehyde (250 mg, 1.35 mmol), 4-Fluoro-3-nitrophenylboronic acid (250 mg, 1.35 mmol), potassium carbonate (560 mg, 4.05 mmol), and Pd(dppf)Cl₂·DCM (110 mg, 0.135 mmol). The product was obtained in 47% yield (156 mg) as a white amorphous solid. ¹H NMR (400 MHz, Chloroform-d) δ 10.12 (s, 1H), 8.35 (dd, J=7.0, 2.4 Hz, 1H), 8.07-8.00 (m, 2H), 7.92 (ddd, J=8.7, 4.1, 2.4 Hz, 1H), 7.80-7.74 (m, 2H), 7.46 (dd, J=10.3, 8.7 Hz, 1H).

### Example 39. Preparation of 5-(((4′-Fluoro-2′-methoxy-[1,1′-biphenyl]-4-yl)methyl)amino)-2-hydroxybenzoic acid

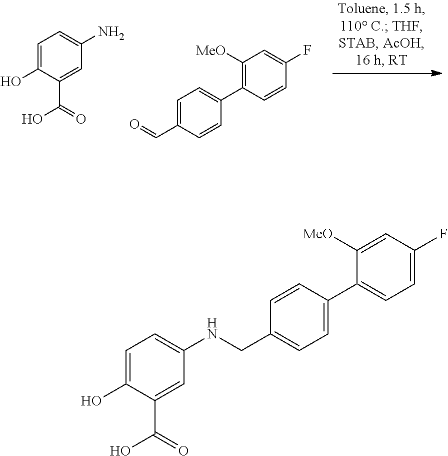 [SMILES: Nc1ccc(O)c(C(=O)O)c1 ; COc1cc(F)ccc1-c1ccc(C=O)cc1 ; COc1cc(F)ccc1-c1ccc(CNc2ccc(O)c(C(=O)O)c2)cc1]

The title compound was synthesized in accordance with General Procedure 3, employing 4′-fluoro-2′-methoxy-[1,1′-biphenyl]-4-carbaldehyde (100 mg, 0.434 mmol) and 5-amino-2-hydroxybenzoic acid (66 mg, 0.434 mmol). Sodium triacetoxyborohydride (184 mg, 0.869 mmol) was employed as the reducing agent. The product was obtained in 30% yield (48 mg) as a white amorphous solid. ¹H NMR (400 MHz, DMSO-d₆) δ 10.52 (s, 1H), 7.39 (s, 4H), 7.29 (dd, J=8.4, 7.0 Hz, 1H), 7.05-6.97 (m, 2H), 6.91 (dd, J=8.9, 3.0 Hz, 1H), 6.84 (td, J=8.4, 2.5 Hz, 1H), 6.74 (d, J=8.9 Hz, 1H), 4.24 (s, 2H), 3.77 (s, 3H). ¹³C NMR (151 MHz, DMSO-d₆) δ 172.5, 163.7, 162.1, 157.9, 157.9, 136.2, 131.7 (d, J=9.9 Hz), 129.6 (2C), 127.5 (2C), 126.5 (d, J=3.2 Hz), 122.3, 117.9, 111.8, 107.4, 107.3, 100.5, 100.3, 56.4, 47.4. HRMS (ESI, m/z) for C₂₁H₁₈FNO₄ [M−H]: expected, 366.1147; found, 396.1131; 4.4 ppm.

### Example 40. Preparation of 5-(((4′-Fluoro-2-methyl-[1,1′-biphenyl]-4-yl)methyl)amino)-2-hydroxybenzoic acid

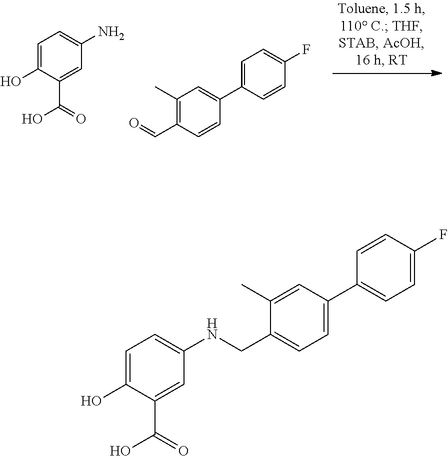 [SMILES: Nc1ccc(O)c(C(=O)O)c1 ; Cc1cc(-c2ccc(F)cc2)ccc1C=O ; Cc1cc(-c2ccc(F)cc2)ccc1CNc1ccc(O)c(C(=O)O)c1]

The title compound was synthesized in accordance with General Procedure 3, employing 4′-fluoro-3-methyl-[1,1′-biphenyl]-4-carbaldehyde (80 mg, 0.37 mmol) and 5-amino-2-hydroxybenzoic acid (57 mg, 0.37 mmol). Sodium triacetoxyborohydride (160 mg, 0.75 mmol) was employed as the reducing agent. The product was obtained in 5% yield (6 mg) as a white amorphous solid. ¹H NMR (400 MHz, DMSO-d₆) δ 10.59 (s, 1H), 7.69 (dd, J=8.8, 5.6 Hz, 2H), 7.48 (d, J=2.0 Hz, 1H), 7.42 (dd, J=7.9, 2.0 Hz, 1H), 7.35 (d, J=7.9 Hz, 1H), 7.27 (t, J=8.9 Hz, 2H), 6.98 (d, J=2.9 Hz, 1H), 6.90 (dd, J=8.9, 3.0 Hz, 1H), 6.75 (d, J=8.8 Hz, 1H), 4.20 (s, 2H), 2.52-2.50 (m, 3H). ¹³C NMR (101 MHz, DMSO-d₆) δ 172.1, 162.9, 160.5, 152.7, 141.5, 137.5, 136.9, 136.5, 128.4 (d, J=8.1 Hz), 128.3, 128.0, 123.9, 121.7, 117.4, 115.6 (d, J=21.2 Hz), 112.5, 110.9, 45.0, 18.7. HRMS (ESI, m/z) for C₂₁H₁₈FNO₃ [M−H]: expected, 350.1198; found, 350.1203; 1.4 ppm.

The intermediate aldehyde was prepared as follows.

### a. 4′-Fluoro-3-methyl-[1,1′-biphenyl]-4-carbaldehyde

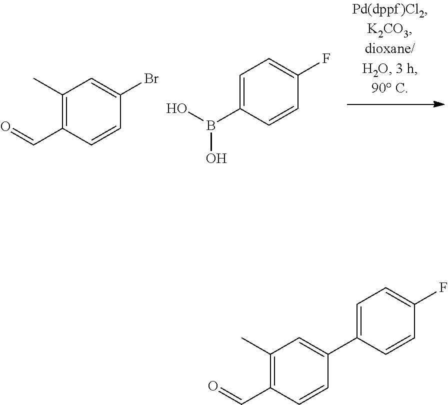

The aldehyde was synthesized in accordance with General Procedure 4, employing 4-bromo-2-methylbenzaldehyde (300 mg, 1.51 mmol), (4-fluorophenyl)boronic acid (211 mg, 1.51 mmol), potassium carbonate (625 mg, 4.52 mmol), and Pd(dppf)Cl₂·DCM (123 mg, 0.151 mmol). The product was obtained in 70% yield (229 mg) as a white amorphous solid. ¹H NMR (400 MHz, Chloroform-d) δ 10.30 (s, 1H), 7.87 (d, J=8.0 Hz, 1H), 7.60 (dd, J=8.8, 5.3 Hz, 2H), 7.54 (dd, J=7.9, 1.9 Hz, 1H), 7.44 (d, J=1.8 Hz, 1H), 7.16 (t, J=8.6 Hz, 2H), 2.74 (s, 3H).

### Example 41. Preparation of 5-(((4′-Fluoro-2′-methyl-[1,1′-biphenyl]-4-yl)methyl)amino)-2-hydroxybenzoic acid

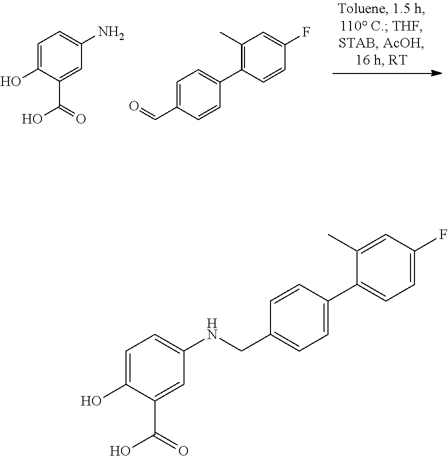 [SMILES: Nc1ccc(O)c(C(=O)O)c1 ; Cc1cc(F)ccc1-c1ccc(C=O)cc1 ; Cc1cc(F)ccc1-c1ccc(CNc2ccc(O)c(C(=O)O)c2)cc1]

The title compound was synthesized in accordance with General Procedure 3, employing 4′-fluoro-2′-methyl-[1,1′-biphenyl]-4-carbaldehyde (80 mg, 0.37 mmol) and 5-amino-2-hydroxybenzoic acid (57 mg, 0.37 mmol). Sodium triacetoxyborohydride (160 mg, 0.75 mmol) was employed as the reducing agent. The product was obtained in 14% yield (19 mg) as a white amorphous solid. ¹H NMR (400 MHz, DMSO-d₆) δ 10.55 (s, 1H), 7.42 (d, J=8.1 Hz, 2H), 7.28 (d, J=8.1 Hz, 2H), 7.21 (dd, J=8.5, 6.1 Hz, 1H), 7.16 (dd, J=10.2, 2.8 Hz, 1H), 7.07 (td, J=8.6, 2.8 Hz, 1H), 7.01 (d, J=2.9 Hz, 1H), 6.92 (dd, J=8.9, 3.0 Hz, 1H), 6.75 (d, J=8.8 Hz, 1H), 4.27 (s, 2H), 2.23 (s, 3H). ¹³C NMR (101 MHz, DMSO-d₆) δ 172.1, 162.4, 160.0, 138.9, 138.6, 137.6 (d, J=8.0 Hz), 137.5 (d, J=2.9 Hz), 131.2 (d, J=8.3 Hz), 129.0 (2C), 127.2 (2C), 121.8, 117.4, 116.7, 116.5, 112.7, 112.4, 111.3, 46.9, 20.3. HRMS (ESI, m/z) for C₂₁H₁₈FNO₃ [M−H]: expected, 350.1198; found, 396.1200; 0.6 ppm.

The intermediate aldehyde was prepared as follows.

### a. 4′-Fluoro-2′-methyl-[1,1′-biphenyl]-4-carbaldehyde

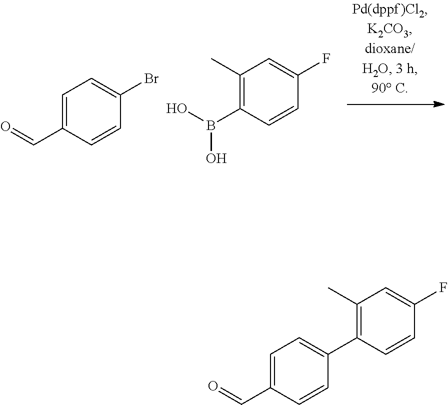

The aldehyde was synthesized in accordance with General Procedure 4, employing 4-bromo-2-methylbenzaldehyde (300 mg, 1.62 mmol), (4-fluoro-2-methylphenyl)boronic acid (211 mg, 1.62 mmol), potassium carbonate (625 mg, 4.86 mmol), and Pd(dppf)Cl₂·DCM (132 mg, 0.162 mmol). The product was obtained in 83% yield (290 mg) as a white amorphous solid. ¹H NMR (400 MHz, Chloroform-d) δ 10.00 (s, 1H), 7.86 (d, J=8.1 Hz, 2H), 7.39 (d, J=8.1 Hz, 2H), 7.12 (dd, J=8.4, 5.9 Hz, 1H), 6.99-6.85 (m, 2H), 2.19 (s, 3H).

### Example 42. Preparation of 5-(((4′-Fluoro-3-methyl-[1,1′-biphenyl]-4-yl)methyl)amino)-2-hydroxybenzoic acid

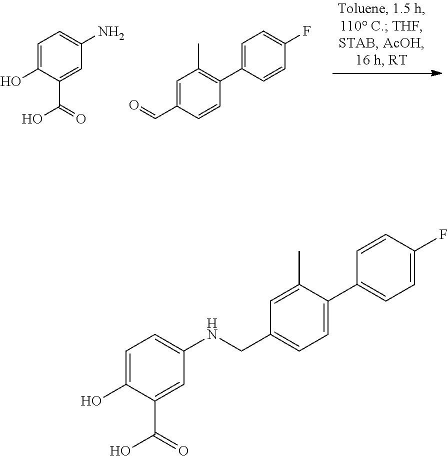 [SMILES: Nc1ccc(O)c(C(=O)O)c1 ; Cc1cc(C=O)ccc1-c1ccc(F)cc1 ; Cc1cc(CNc2ccc(O)c(C(=O)O)c2)ccc1-c1ccc(F)cc1]

The title compound was synthesized in accordance with General Procedure 3, employing 4′-fluoro-2-methyl-[1,1′-biphenyl]-4-carbaldehyde (80 mg, 0.37 mmol) and 5-amino-2-hydroxybenzoic acid (57 mg, 0.37 mmol). Sodium triacetoxyborohydride (160 mg, 0.75 mmol) was employed as the reducing agent. After column chromatography, precipitate was collected and filtered. The product was obtained in 4% yield (5 mg) as a white amorphous solid. ¹H NMR (400 MHz, DMSO-d₆) δ 7.36 (dd, J=8.6, 5.6 Hz, 2H), 7.31-7.22 (m, 4H), 7.15 (d, J=7.8 Hz, 1H), 7.00 (d, J=2.9 Hz, 1H), 6.91 (dd, J=8.8, 3.0 Hz, 1H), 6.75 (d, J=8.9 Hz, 1H), 4.22 (s, 2H), 2.21 (s, 3H). ¹³C NMR (101 MHz, DMSO-d₆) δ 172.1, 162.9, 160.5, 152.7, 141.5, 137.5-137.4 (m), 136.7 (d, J=34.2 Hz), 136.5, 128.5, 128.4, 128.3, 128.0, 123.9, 121.7, 117.4, 115.7, 115.5, 112.5, 110.9, 45.0, 18.7. HRMS (ESI, m/z) for C₂₁H₁₈FNO₃ [M−H]: expected, 350.1198; found, 350.1205; 2.0 ppm.

The intermediate aldehyde was prepared as follows.

### a. 4′-Fluoro-2-methyl-[1,1′-biphenyl]-4-carbaldehyde

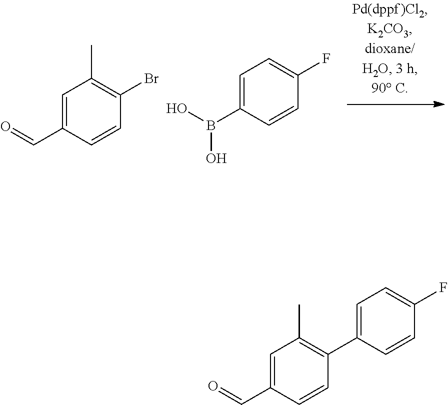

The aldehyde was synthesized in accordance with General Procedure 4, employing 4-bromo-3-methylbenzaldehyde (300 mg, 1.51 mmol), (4-fluorophenyl)boronic acid (211 mg, 1.51 mmol), potassium carbonate (625 mg, 4.52 mmol), and Pd(dppf)Cl₂·DCM (123 mg, 0.151 mmol). The product was obtained in 91% yield (294 mg) as a white amorphous solid. ¹H NMR (400 MHz, Chloroform-d) δ 9.91 (s, 1H), 7.68 (d, J=1.7 Hz, 1H), 7.26 (d, J=7.8 Hz, 1H), 7.24-7.17 (m, 1H), 7.03 (t, J=8.7 Hz, 2H), 2.23 (s, 3H).

### Example 43. Preparation of 5-(((4′-Fluoro-[1,1′-biphenyl]-4-yl)methyl)amino)-2-methoxybenzonitrile

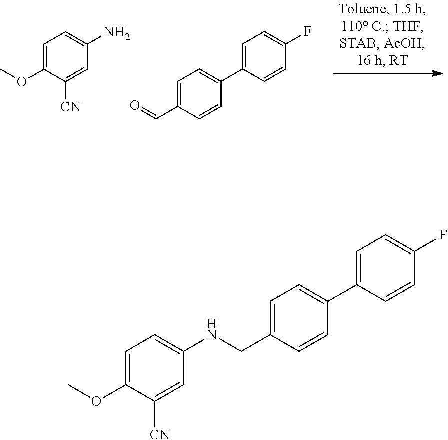 [SMILES: COc1ccc(N)cc1C#N ; O=Cc1ccc(-c2ccc(F)cc2)cc1 ; COc1ccc(NCc2ccc(-c3ccc(F)cc3)cc2)cc1C#N]

The title compound was synthesized in accordance with General Procedure 3, employing 4′-fluoro-[1,1′-biphenyl]-4-carbaldehyde (150 mg, 0.749 mmol) and 5-amino-2-methoxybenzonitrile (111 mg, 0.749 mmol). Sodium triacetoxyborohydride (318 mg, 1.5 mmol) was employed as the reducing agent. The product was obtained in 61% yield (153 mg) as a white amorphous solid. ¹H NMR (400 MHz, DMSO-d₆) δ 7.69 (dd, J=8.8, 5.5 Hz, 2H), 7.61 (d, J=8.2 Hz, 2H), 7.43 (d, J=8.1 Hz, 2H), 7.28 (t, J=8.9 Hz, 2H), 7.02 (d, J=9.2 Hz, 1H), 6.93 (dd, J=9.1, 2.9 Hz, 1H), 6.83 (d, J=2.9 Hz, 1H), 6.35 (t, J=6.1 Hz, 1H), 4.29 (d, J=6.1 Hz, 2H), 3.76 (s, 3H). ¹³C NMR (101 MHz, DMSO-d₆) δ 161.7 (d, J=244.1 Hz), 152.3, 142.9, 139.0, 137.6, 136.4 (d, J=3.1 Hz), 128.5 (d, J=8.1 Hz) (2C), 127.8 (2C), 126.6 (2C), 119.2, 117.0, 115.7 (d, J=21.3 Hz) (2C), 115.0, 113.5, 100.1, 56.2, 46.2. HRMS (ESI, m/z) for C₂₀H₁₄FNO₄ [M+Na]: expected, 355.1217; found, 355.1218; 0.3 ppm.

### Example 44. Preparation of 5-(((4′-Fluoro-3′-methyl-[1,1′-biphenyl]-4-yl)methyl)amino)-2-hydroxybenzoic acid

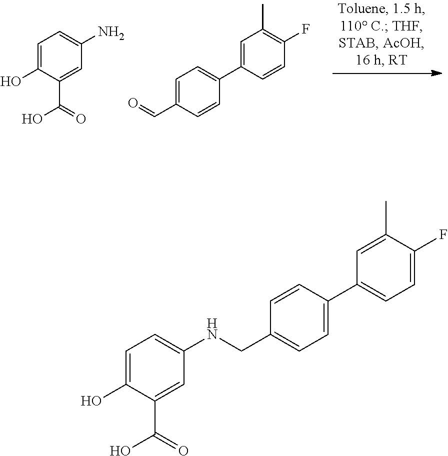 [SMILES: Nc1ccc(O)c(C(=O)O)c1 ; Cc1cc(-c2ccc(C=O)cc2)ccc1F ; Cc1cc(-c2ccc(CNc3ccc(O)c(C(=O)O)c3)cc2)ccc1F]

The title compound was synthesized in accordance with General Procedure 3, employing 4′-fluoro-2′-methyl-[1,1′-biphenyl]-4-carbaldehyde (80 mg, 0.37 mmol) and 5-amino-2-hydroxybenzoic acid (57 mg, 0.37 mmol). Sodium triacetoxyborohydride (160 mg, 0.75 mmol) was employed as the reducing agent. The product was obtained in 3% yield (4 mg) as a white amorphous solid. ¹H NMR (400 MHz, DMSO-d₆) δ 10.61 (s, 1H), 7.61-7.56 (m, 3H), 7.49 (ddd, J=8.0, 5.0, 2.5 Hz, 1H), 7.43 (d, J=8.1 Hz, 2H), 7.20 (dd, J=9.7, 8.5 Hz, 1H), 6.98 (d, J=2.9 Hz, 1H), 6.90 (dd, J=8.9, 3.0 Hz, 1H), 6.73 (d, J=8.8 Hz, 1H), 4.26 (s, 2H), 2.29 (d, J=1.9 Hz, 3H). ¹³C NMR (151 MHz, DMSO-d₆) δ 172.5, 161.6, 160.0, 153.3, 141.7, 139.8, 138.2, 136.6 (d, J=3.4 Hz), 130.3 (d, J=5.2 Hz), 128.3 (2C), 127.0 (2C), 126.2 (d, J=8.1 Hz), 125.0 (d, J=17.3 Hz), 122.3, 117.9, 115.7 (d, J=22.1 Hz), 12.5 (d, J=176.0 Hz), 47.3, 14.7. HRMS (ESI, m/z) for C₂₁H₁₈FNO₃ [M−H]: expected, 350.1198; found, 350.1203; 1.4 ppm.

The intermediate aldehyde was prepared as follows.

### a. 4′-Fluoro-3′-methyl-[1,1′-biphenyl]-4-carbaldehyde

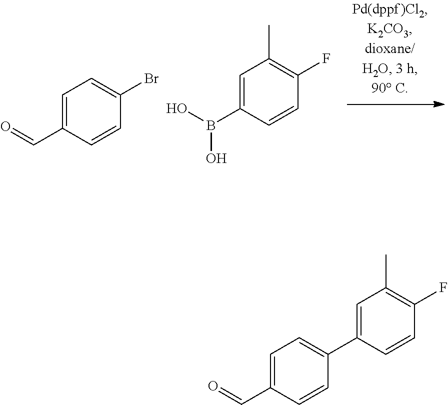

The aldehyde was synthesized in accordance with General Procedure 4, employing 4-bromobenzaldehyde (300 mg, 1.62 mmol), (4-fluoro-3-methylphenyl)boronic acid (250 mg, 1.62 mmol), potassium carbonate (672 mg, 4.86 mmol), and Pd(dppf)Cl₂·DCM (132 mg, 0.162 mmol). The product was obtained in 91% yield (317 mg) as a white amorphous solid. ¹H NMR (400 MHz, Chloroform-d) δ 10.03 (s, 1H), 7.92 (d, J=8.0 Hz, 2H), 7.67 (d, J=8.0 Hz, 2H), 7.41 (ddt, J=13.2, 5.2, 2.4 Hz, 2H), 7.08 (t, J=8.9 Hz, 1H), 2.34 (d, J=2.1 Hz, 3H).

### Example 45. Preparation of 3-(((4′-Fluoro-[1,1′-biphenyl]-4-yl)methyl)amino)-5-hydroxybenzoic acid

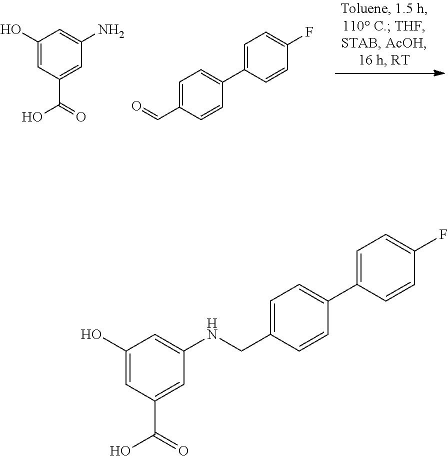 [SMILES: Nc1cc(O)cc(C(=O)O)c1 ; O=Cc1ccc(-c2ccc(F)cc2)cc1 ; O=C(O)c1cc(O)cc(NCc2ccc(-c3ccc(F)cc3)cc2)c1]

The title compound was synthesized in accordance with General Procedure 3, employing 4′-fluoro-[1,1′-biphenyl]-4-carbaldehyde (131 mg, 0.653 mmol) and 3-amino-5-hydroxybenzoic acid (100 mg, 0.653 mmol). Sodium triacetoxyborohydride (277 mg, 1.31 mmol) was employed as the reducing agent. The product was obtained in 77 mg (70% yield) as a white amorphous solid. ¹H NMR (400 MHz, Methanol-d₄) δ 7.61 (dd, J=8.7, 5.4 Hz, 2H), 7.54 (d, J=8.3 Hz, 2H), 7.44 (d, J=7.9 Hz, 2H), 7.14 (t, J=8.8 Hz, 2H), 6.88-6.85 (m, 1H), 6.74-6.70 (m, 1H), 6.28 (t, J=2.2 Hz, 1H), 4.36 (s, 2H). ¹³C NMR (101 MHz, Methanol-d₄) δ 170.9, 165.0, 162.6, 159.3, 151.4, 140.5, 140.0, 138.7 (d, J=3.2 Hz), 133.6, 129.7 (d, J=8.1 Hz) (2C), 128.8 (2C), 128.0 (2C), 116.5 (d, J=21.7 Hz) (2C), 107.5, 106.2, 104.8. HRMS (ESI, m/z) for C₂₀H₁₆FNO₃ [M−H]: expected, 336.1141; found, 337.1044; 0.9 ppm.

### Example 46. Preparation of 5-(((4′-fluoro-[1,1′-biphenyl]-4-yl)methyl)amino)-2-methoxybenzoic acid

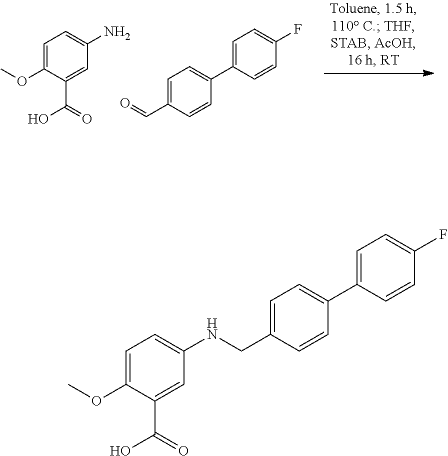

The title compound was synthesized in accordance with General Procedure 1, employing 4′-fluoro-[1,1′-biphenyl]-4-carbaldehyde (120 mg, 0.598 mmol) and 5-amino-2-methoxybenzoic acid (100 mg. 0.598 mmol). Sodium triacetoxyborohydride (254 mg, 1.20 mmol) was employed as the reducing agent. The product was obtained in 31 mg (15% yield) as a white amorphous solid. ¹H NMR (400 MHz, Methanol-d₄) δ 7.63-7.57 (m, 2H), 7.53 (d, J=8.2 Hz, 2H), 7.44 (d, J=8.2 Hz, 2H), 7.18 (d, J=3.0 Hz, 1H), 7.14 (t, J=8.8 Hz, 2H), 6.94 (d, J=8.9 Hz, 1H), 6.85 (dd, J=8.9, 3.0 Hz, 1H), 4.34 (s, 2H), 3.83 (s, 3H). ¹³C NMR (101 MHz, Methanol-d₄) δ 170.2, 165.0, 162.6, 152.3, 144.3, 140.4, 140.1, 138.6 (d, J=3.2 Hz), 129.7 (d, J=8.1 Hz) (2C), 129.0 (2C), 128.0 (2C), 121.5, 119.7, 116.9, 116.5 (d, J=21.6 Hz) (2C), 115.1, 57.3. HRMS (ESI, m/z) for C₂₁H₁₈FNO₃ [M−H]: expected, 351.1276; found, 351.1278; 0.6 ppm.

### Example 47. Preparation of 5-(((4′-Fluoro-[1,1′-biphenyl]-4-yl)methyl)amino)-2-hydroxybenzonitrile

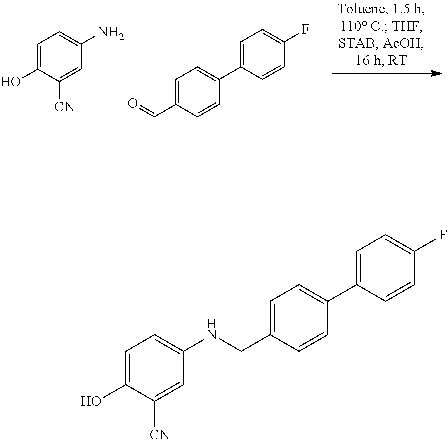 [SMILES: N#Cc1cc(N)ccc1O ; O=Cc1ccc(-c2ccc(F)cc2)cc1 ; N#Cc1cc(NCc2ccc(-c3ccc(F)cc3)cc2)ccc1O]

The title compound was synthesized in accordance with General Procedure 3, employing 4′-fluoro-[1,1′-biphenyl]-4-carbaldehyde (75 mg, 0.37 mmol) and 5-amino-2-hydroxybenzonitrile (50 mg, 0.37 mmol). Sodium triacetoxyborohydride (160 mg, 0.75 mmol) was employed as the reducing agent. The product was obtained in 30% yield (36 mg) as a white amorphous solid. ¹H NMR (400 MHz, DMSO-d₆) δ 10.02 (s, 1H), 7.69 (dd, J=8.8, 5.5 Hz, 2H), 7.61 (d, J=8.3 Hz, 2H), 7.43 (d, J=8.1 Hz, 2H), 7.28 (t, J=8.9 Hz, 2H), 6.85 (dd, J=9.0, 2.8 Hz, 1H), 6.79 (d, J=9.0 Hz, 1H), 6.66 (d, J=2.8 Hz, 1H), 6.14 (t, J=6.1 Hz, 1H), 4.25 (d, J=6.0 Hz, 2H). ¹³C NMR (101 MHz, DMSO-d₆) δ 161.7 (d, J=244.1 Hz), 151.3, 141.7, 139.2, 137.6, 136.4 (d, J=2.9 Hz), 128.5 (d, J=8.0 Hz) (2C), 127.9 (2C), 126.6 (2C), 120.4, 117.7, 117.2, 115.6 (d, J=21.3 Hz) (2C), 113.6, 98.4, 46.5. HRMS (ESI, m/z) for C₂₀H₁₅FN₂O [M−H]: expected, 317.1096; found, 317.1106; 3.2 ppm.

### Example 48. Preparation of 5-(((4′-Fluoro-[1,1′-biphenyl]-4-yl)methyl)amino)-2-hydroxybenzonitrile

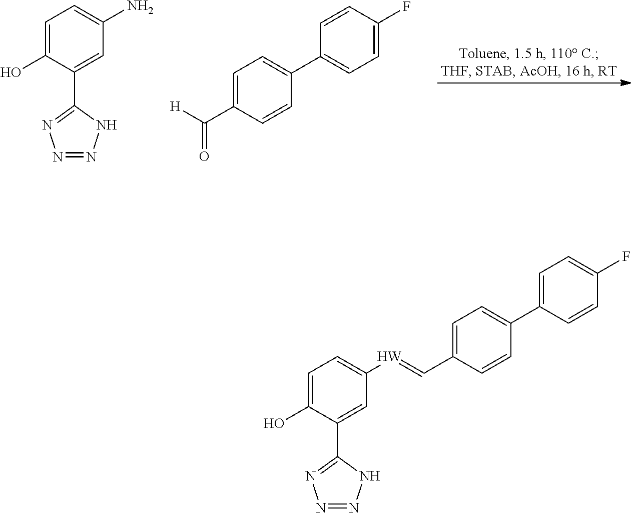

The title compound was synthesized in accordance with General Procedure 3, employing 4′-fluoro-[1,1′-biphenyl]-4-carbaldehyde (76 mg, 0.38 mmol) and 4-amino-2-(2H-tetrazol-5-yl)phenol (67 mg, 0.38 mmol). Sodium triacetoxyborohydride (160 mg, 0.76 mmol) was employed as the reducing agent. The product was obtained in 15% yield (20 mg) as a tan amorphous solid. ¹H NMR (400 MHz, DMSO-d₆) δ 15.75 (s, 1H), 10.00 (s, 1H), 7.73-7.66 (m, 2H), 7.61 (d, J=8.3 Hz, 2H), 7.47 (d, J=8.3 Hz, 2H), 7.31-7.24 (m, 3H), 6.85 (d, J=8.9 Hz, 1H), 6.76 (dd, J=8.8, 2.9 Hz, 1H), 6.09 (d, J=32.4 Hz, 1H), 4.30 (s, 2H). ¹³C NMR (214 MHz, DMSO-d₆) δ 162.8, 161.7, 147.5 (d, J=2189.2 Hz), 147.0, 140.0, 138.0, 136.9 (d, J=3.0 Hz), 129.0 (d, J=8.4 Hz), 128.4, 127.0, 118.4, 117.6, 116.1 (d, J=21.6 Hz), 111.6, 110.8, 47.3. HRMS (ESI, m/z) for C₂₀H₁₃FN₂O [M−H]: expected, 360.1266; found, 360.1251; 4.2 ppm.

The intermediate alcohol was prepared as follows.

### a. 4-Nitro-2-(2H-tetrazol-5-yl)phenol

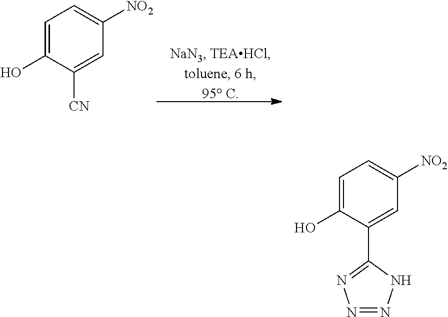 [SMILES: N#Cc1cc([N+](=O)[O-])ccc1O ; O=[N+]([O-])c1ccc(O)c(-c2nnn[nH]2)c1]

Sodium azide (1.188 g, 18.28 mmol), triethylamine hydrochloride (2.531 g, 18.28 mmol), and 5-nitro-2-hydroxybenzonitrile (1 g, 6.09 mmol) were stirred in 10 mL toluene for 6 hr at 95° C. The reaction was cooled to room temperature and extracted with 20 mL deionized water. Ice was added to the aqueous phase and the aqueous phase was acidified dropwise with 12 M aq. HCl. The suspension was stirred for 15 min, then filtered and the precipitate collected. After filtration, the precipitate was dried under reduced pressure by rotary evaporation. The product was obtained in 96% yield as a tan amorphous solid (1.213 g). ¹H NMR (400 MHz, DMSO-d₆) δ 8.83 (d, J=2.9 Hz, 1H), 8.32 (d, J=9.1 Hz, 1H), 7.24 (d, J=9.2 Hz, 1H).

### b. 4-Amino-2-(2H-tetrazol-5-yl)phenol

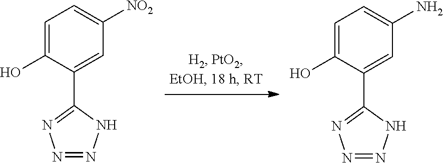 [SMILES: O=[N+]([O-])c1ccc(O)c(-c2nnn[nH]2)c1 ; Nc1ccc(O)c(-c2nnn[nH]2)c1]

4-nitro-2-(2H-tetrazol-5-yl)phenol (1.013 g, 4.895 mmol) was stirred at room temperature in 10 mL EtOH. PtO₂ (55.6 mg, 0.245 mmol) was added, and hydrogen gas bubbled through the mixture for 5 min. The reaction mixture was stirred for 12 hr at room temperature under 1 atm of hydrogen gas. The solvent was removed by rotary evaporation and the crude residue purified by flash column chromatography (SiO₂, eluted with a gradient of 0-50% MeOH in DCM). The product was obtained in 20% yield as a brown amorphous solid (170 mg). ¹H NMR (400 MHz, DMSO-d₆) δ 7.32 (d, J=2.8 Hz, 1H), 6.69 (d, J=8.5 Hz, 1H), 6.56 (dd, J=8.6, 2.8 Hz, 1H).

### Example 49. Biological Assays

The biological activity of compounds of the invention can be evaluated using known assays and using the assays described below.

### Methods

Cell-based Luciferase Assay. Agonist activity of test compounds against human PPAR isoforms, was analyzed using commercial kits from Indigo Biosciences (hPPARα: IB00111, hPPARγ: IB00101: hPPARδ: IB00121). Protocols provided with the kit were followed for experimental execution. Briefly, a suspension of reporter cells was prepared in Cell Recovery Medium (CRM: containing 10% charcoal stripped fetal bovine serum (FBS)). 200 μL of the reporter cell suspension was dispensed into wells of white 96-well assay plates provided within the kit for a 4-6 hours pre-incubation period at 37° C. and 5% CO₂. At the end of the pre-incubation period, the culture media was discarded and 200 μL of Compound Screening Medium (CSM: containing 10% charcoal stripped FBS) containing the requisite concentration of derivative to be evaluated was added to representative wells. Following a 22-24 hours incubation at 37° C. and 5% CO₂, the treatment media were discarded and 100 μL of the provided Luciferase Detection Reagent was added to each well. Luminescence was quantified using a GloMax Explorer (Promega). For select compounds, dose-response analyses were performed via non-linear curve-fitting with four parameters. Plots of relative light units (RLU) vs. Log[compound, (μM)] were generated using GraphPad Prism software, which was also used to calculate EC₅₀ and SEM values. Unless otherwise noted, each compound evaluated was tested in three separate experiments in triplicate. Data for representative compounds is provided in the following table. The % at each concentration is the percent relative fluorescent units (RFU) produced by the noted concentration of compound with respect to 90 nM of GW7647, the positive control in the PPAR-alpha luciferase assay. 90 nM of GW7647 is ~10× the EC50 and produces full agonism without signs of cytotoxicity. Data for representative compounds is shown in the following Table.

% PPAR-alpha AGONISM

| Compound | Example | % at 0.5 μm | % at 5 μm | % at 50 μm |
|---|---|---|---|---|
| 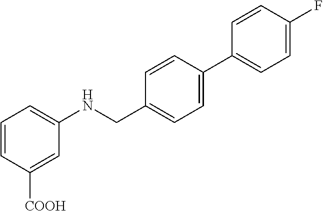 [SMILES: O=C(O)c1cccc(NCc2ccc(-c3ccc(F)cc3)cc2)c1] | 1 | 35.6% | 124.7% | 55.1% |
| 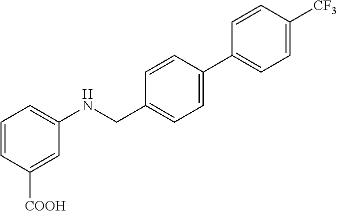 [SMILES: O=C(O)c1cccc(NCc2ccc(-c3ccc(C(F)(F)F)cc3)cc2)c1] | 2 | 74.7% | 102.9% | 27.4% |
| 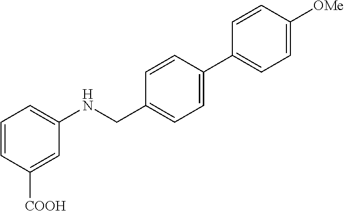 [SMILES: COc1ccc(-c2ccc(CNc3cccc(C(=O)O)c3)cc2)cc1] | 3 | 32.9% | 111.5% | 98.3% |
| 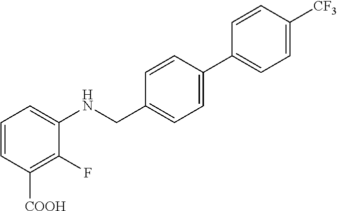 [SMILES: O=C(O)c1cccc(NCc2ccc(-c3ccc(C(F)(F)F)cc3)cc2)c1F] | 4 | 73.7% | 120.2% | 20.9% |
| 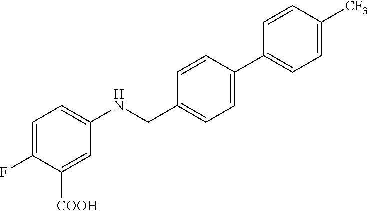 [SMILES: O=C(O)c1cc(NCc2ccc(-c3ccc(C(F)(F)F)cc3)cc2)ccc1F] | 5 | 67.4% | 92.5% | 54.4% |
| 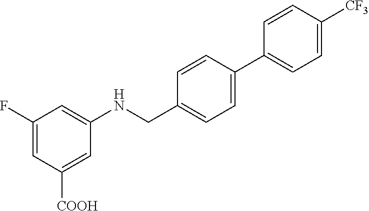 [SMILES: O=C(O)c1cc(F)cc(NCc2ccc(-c3ccc(C(F)(F)F)cc3)cc2)c1] | 6 | 91.8% | 122.3% | 54.3% |
| 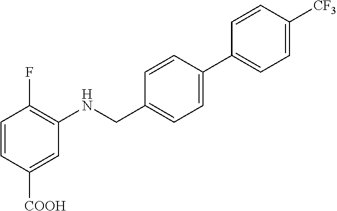 [SMILES: O=C(O)c1ccc(F)c(NCc2ccc(-c3ccc(C(F)(F)F)cc3)cc2)c1] | 7 | 6.6% | 98.0% | 68.1% |
| 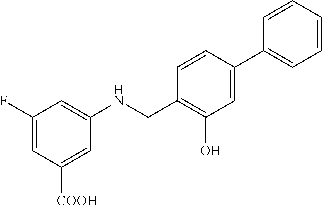 [SMILES: O=C(O)c1cc(F)cc(NCc2ccc(-c3ccccc3)cc2O)c1] | 8 | 17.6% | 50.3% | 95.2% |
| 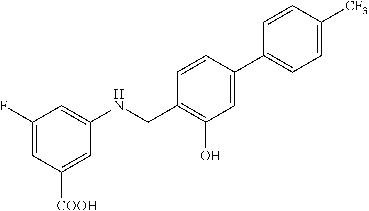 [SMILES: O=C(O)c1cc(F)cc(NCc2ccc(-c3ccc(C(F)(F)F)cc3)cc2O)c1] | 9 | 8.0% | 49.6% | 46.8% |
| 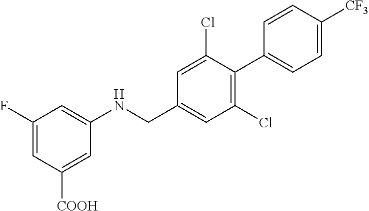 [SMILES: O=C(O)c1cc(F)cc(NCc2cc(Cl)c(-c3ccc(C(F)(F)F)cc3)c(Cl)c2)c1] | 10 | 0.0% | 37.2% | 17.4% |
| 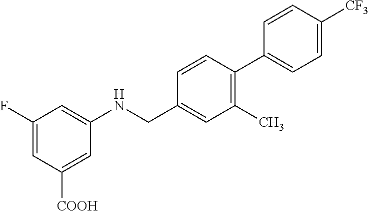 [SMILES: Cc1cc(CNc2cc(F)cc(C(=O)O)c2)ccc1-c1ccc(C(F)(F)F)cc1] | 11 | 84.0% | 55.0% | 36.0% |
| 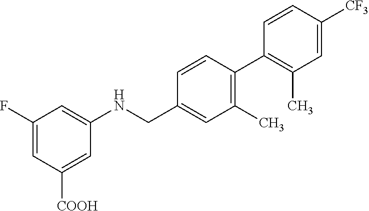 [SMILES: Cc1cc(CNc2cc(F)cc(C(=O)O)c2)ccc1-c1ccc(C(F)(F)F)cc1C] | 12 | 14.1% | 46.3% | 26.5% |
| 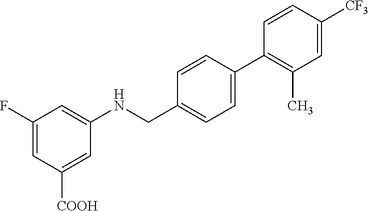 [SMILES: Cc1cc(C(F)(F)F)ccc1-c1ccc(CNc2cc(F)cc(C(=O)O)c2)cc1] | 13 | 38.3% | 47.4% | 27.5% |
| 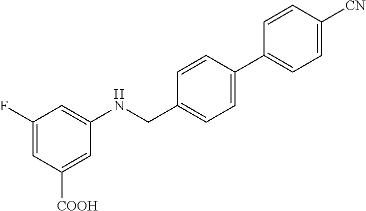 [SMILES: N#Cc1ccc(-c2ccc(CNc3cc(F)cc(C(=O)O)c3)cc2)cc1] | 14 | 7.0% | 58.7% | 83.8% |
| 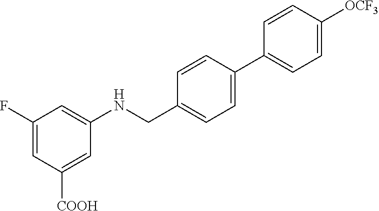 [SMILES: O=C(O)c1cc(F)cc(NCc2ccc(-c3ccc(OC(F)(F)F)cc3)cc2)c1] | 15 | 55.0% | 118.1% | 98.2% |
| 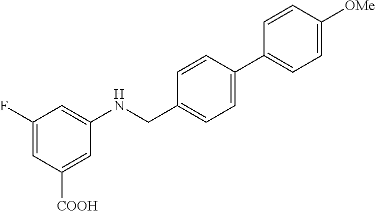 [SMILES: COc1ccc(-c2ccc(CNc3cc(F)cc(C(=O)O)c3)cc2)cc1] | 16 | 38.2% | 106.5% | 96.0% |
| 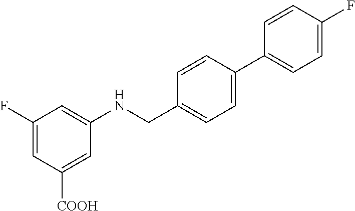 [SMILES: O=C(O)c1cc(F)cc(NCc2ccc(-c3ccc(F)cc3)cc2)c1] | 17 | 47.6% | 85.3% | 100.4% |
| 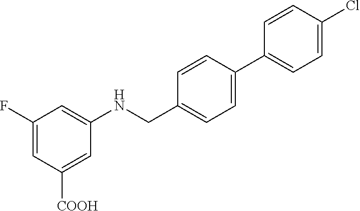 [SMILES: O=C(O)c1cc(F)cc(NCc2ccc(-c3ccc(Cl)cc3)cc2)c1] | 18 | 38.6% | 80.0% | 87.8% |
| 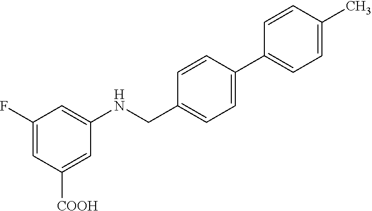 [SMILES: Cc1ccc(-c2ccc(CNc3cc(F)cc(C(=O)O)c3)cc2)cc1] | 19 | 40.6% | 101.3% | 98.9% |
| 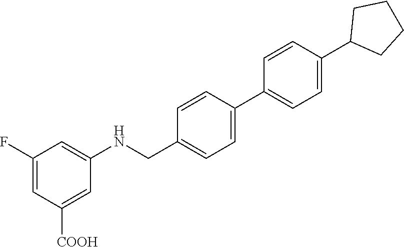 [SMILES: O=C(O)c1cc(F)cc(NCc2ccc(-c3ccc(C4CCCC4)cc3)cc2)c1] | 20 | 38.2% | 58.5% | 48.0% |
| 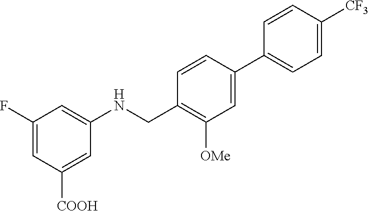 [SMILES: COc1cc(-c2ccc(C(F)(F)F)cc2)ccc1CNc1cc(F)cc(C(=O)O)c1] | 21 | 0.0% | 17.5% | 4.8% |
| 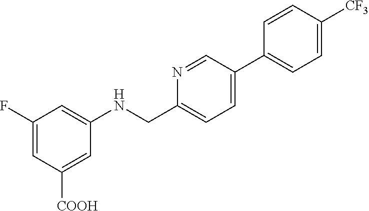 [SMILES: O=C(O)c1cc(F)cc(NCc2ccc(-c3ccc(C(F)(F)F)cc3)cn2)c1] | 22 | 22.1% | 62.2% | 42.1% |
| 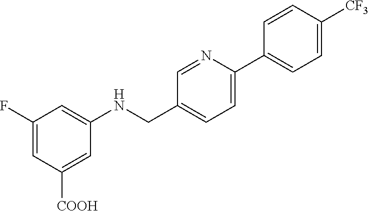 [SMILES: O=C(O)c1cc(F)cc(NCc2ccc(-c3ccc(C(F)(F)F)cc3)nc2)c1] | 23 | 51.0% | 50.3% | 45.9% |
| 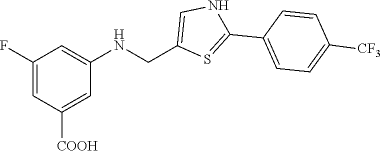 | 24 | 15.7% | 83.3% | 74.9% |
| 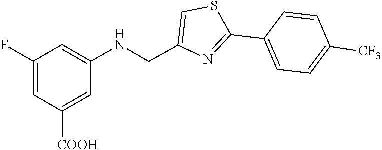 [SMILES: O=C(O)c1cc(F)cc(NCc2csc(-c3ccc(C(F)(F)F)cc3)n2)c1] | 25 | 21.7% | 88.7% | 42.4% |
| 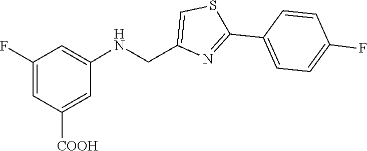 [SMILES: O=C(O)c1cc(F)cc(NCc2csc(-c3ccc(F)cc3)n2)c1] | 26 | 10.6% | 14.2% | 50.0% |
| 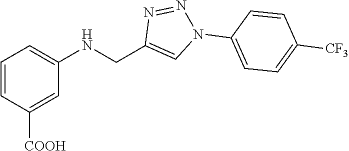 [SMILES: O=C(O)c1cccc(NCc2cn(-c3ccc(C(F)(F)F)cc3)nn2)c1] | 27 | 51.1% | 94.3% | 58.5% |
| 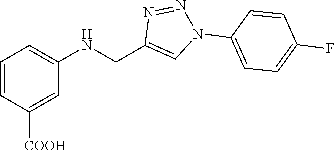 [SMILES: O=C(O)c1cccc(NCc2cn(-c3ccc(F)cc3)nn2)c1] | 28 | 13.1% | 21.5% | 58.6% |
| 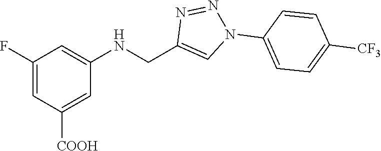 [SMILES: O=C(O)c1cc(F)cc(NCc2cn(-c3ccc(C(F)(F)F)cc3)nn2)c1] | 29 | 27.0% | 138.7% | 91.0% |
| 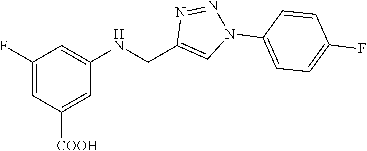 [SMILES: O=C(O)c1cc(F)cc(NCc2cn(-c3ccc(F)cc3)nn2)c1] | 30 | 9.6% | 19.1% | 48.8% |
| 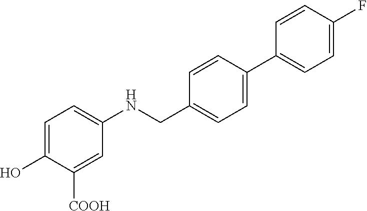 [SMILES: O=C(O)c1cc(NCc2ccc(-c3ccc(F)cc3)cc2)ccc1O] | 31 | 39.4% | 196.7% | 3.1% |

IRF-Lucia Reporter STING Inhibition Assay. THP-1 Lucia ISG cells (InvivoGen, thpl-isg) were seeded in white opaque 96-well plates (SPL, 30196) at 5.0×10⁵ cells/mL to a final volume of 200 μL. Cells were incubated at 37° C. in a 5% CO₂ atmosphere with varying concentrations of test compound for 1.5 hours, followed by treatment with 1 μM 2,3-cGAMP (InvivoGen, tlrl-nacga23-02) and incubated for 20 hours. A 20 μL aliquot of cell suspension was added to 50 μL. QUANTI-Luc™ reagent (InvivoGen, rep-qlc4lg2), and the resulting luminescence is measured by a GloMax Explorer (Promega) with a 0.1 second read time. Data for representative compounds is shown in the following Table.

% STING INHIBITION

| Compound | Example | % at 12.5 μm | % at 25 μm | % at 50 μm |
|---|---|---|---|---|
| 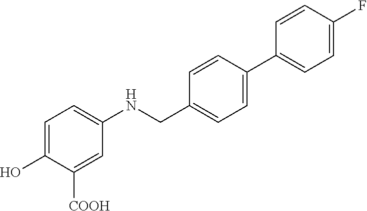 [SMILES: O=C(O)c1cc(NCc2ccc(-c3ccc(F)cc3)cc2)ccc1O] | 31 | 69% | 96% | 97% |
| 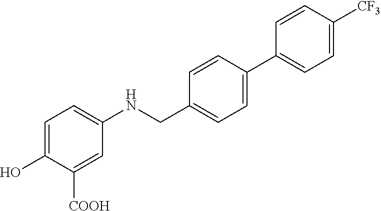 [SMILES: O=C(O)c1cc(NCc2ccc(-c3ccc(C(F)(F)F)cc3)cc2)ccc1O] | 32 | 28% | 43% | 64% |
| 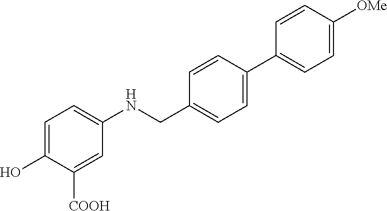 [SMILES: COc1ccc(-c2ccc(CNc3ccc(O)c(C(=O)O)c3)cc2)cc1] | 33 | 0% | 0% | 0% |
| 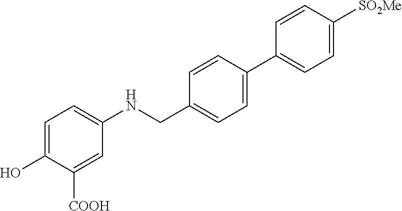 [SMILES: CS(=O)(=O)c1ccc(-c2ccc(CNc3ccc(O)c(C(=O)O)c3)cc2)cc1] | 34 | 0% | 0% | 0% |
| 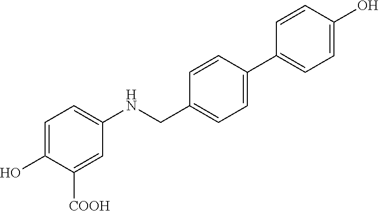 [SMILES: O=C(O)c1cc(NCc2ccc(-c3ccc(O)cc3)cc2)ccc1O] | 35 | 0% | 0% | 0% |
| 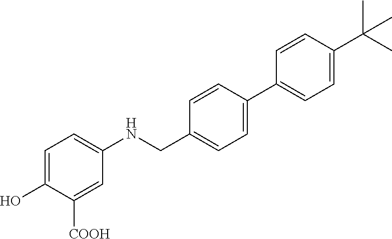 [SMILES: CC(C)(C)c1ccc(-c2ccc(CNc3ccc(O)c(C(=O)O)c3)cc2)cc1] | 36 | 0% | 0% | >95% |
| 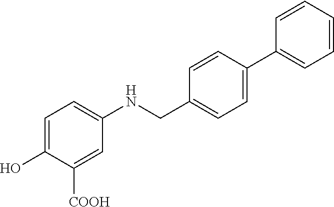 [SMILES: O=C(O)c1cc(NCc2ccc(-c3ccccc3)cc2)ccc1O] | 37 | 0% | 28% | 66% |
| 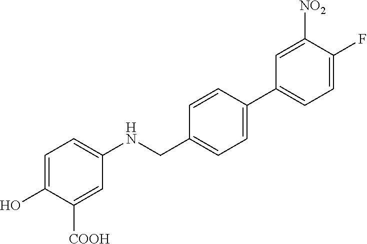 [SMILES: O=C(O)c1cc(NCc2ccc(-c3ccc(F)c([N+](=O)[O-])c3)cc2)ccc1O] | 38 | 0% | 39% | 90% |
| 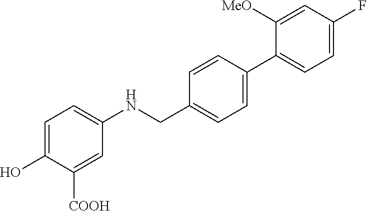 [SMILES: COc1cc(F)ccc1-c1ccc(CNc2ccc(O)c(C(=O)O)c2)cc1] | 39 | 0% | 0% | 0% |
| 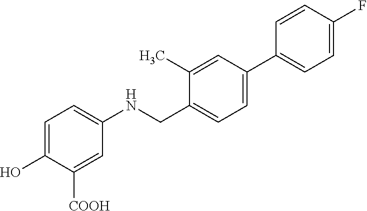 [SMILES: Cc1cc(-c2ccc(F)cc2)ccc1CNc1ccc(O)c(C(=O)O)c1] | 40 | 33% | 24% | 52% |
| 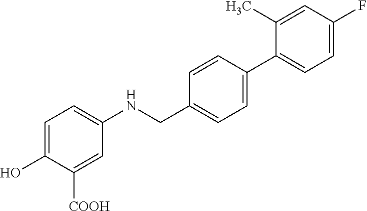 [SMILES: Cc1cc(F)ccc1-c1ccc(CNc2ccc(O)c(C(=O)O)c2)cc1] | 41 | 25% | 47% | 80% |
| 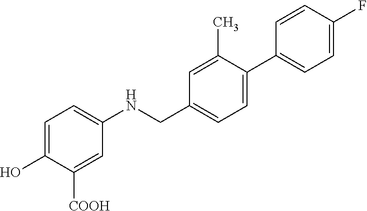 [SMILES: Cc1cc(CNc2ccc(O)c(C(=O)O)c2)ccc1-c1ccc(F)cc1] | 42 | 0% | 37% | 76% |
| 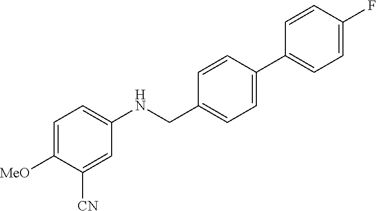 [SMILES: COc1ccc(NCc2ccc(-c3ccc(F)cc3)cc2)cc1C#N] | 43 | 0% | 0% | 0% |
| 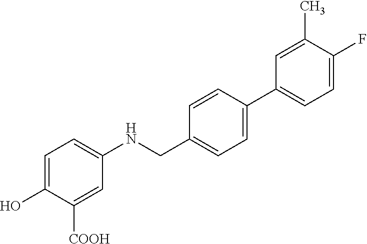 [SMILES: Cc1cc(-c2ccc(CNc3ccc(O)c(C(=O)O)c3)cc2)ccc1F] | 44 | 0% | 12% | 81% |
| 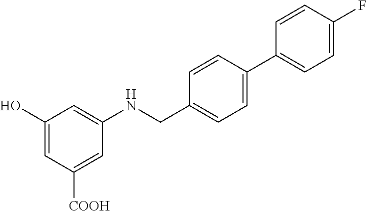 [SMILES: O=C(O)c1cc(O)cc(NCc2ccc(-c3ccc(F)cc3)cc2)c1] | 45 | 9% | 50% | 80% |
| 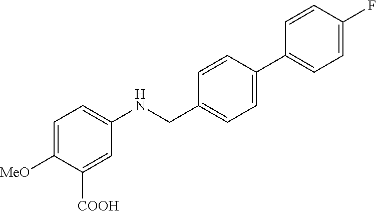 [SMILES: COc1ccc(NCc2ccc(-c3ccc(F)cc3)cc2)cc1C(=O)O] | 46 | 9% | 31% | 53% |
| 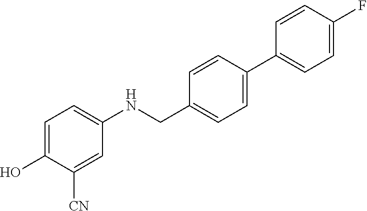 [SMILES: N#Cc1cc(NCc2ccc(-c3ccc(F)cc3)cc2)ccc1O] | 47 | 40% | 80% | 99% |
| 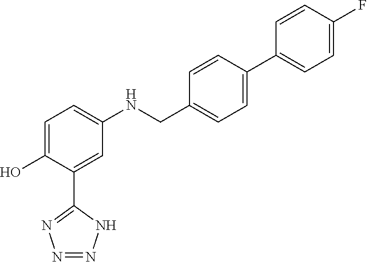 [SMILES: Oc1ccc(NCc2ccc(-c3ccc(F)cc3)cc2)cc1-c1nnn[nH]1] | 48 | 0% | 32% | 77% |

qRT-PCR Human primary RPE cells were treated with 25 μM of Example 17 (A229) or fenofibric acid (FA) for 24 hours. The cells were then harvested and lysed in Trizol reagent (Invitrogen, Waltham, MA). RNA was extracted using an RNeasy kit (QIAGEN, Germantown, MD). cDNA was generated with the reverse transcriptase reaction. mRNA levels of target genes were measured by qRT-PCR with the primers, including downstream target genes of PPARα as follows: acyl-CoA dehydrogenase medium chain (Acadm), carnitine palmitoyl transferase 1A (Cpt1a), fatty acid binding protein 3 (Fabp3), and solute carrier family 25 member 20 (Slc25a20), and two mitochondrial biogenesis genes: silent information regulator-1 (SIRT1) and peroxisome proliferator activated receptor γ co-activator 1α (PGC-1α).

hRPE Cell Viability Assessment. Human primary RPE cells were treated with 25 μM of Example 17 (A229) or FA for 4 hours, then 150 μM palmitate (PA) was added and co-incubated with the cells for another 24 hours. The resulting cell suspension was then stained with trypan blue (Thermo Fisher). Viable cells were counted using Cellometer Auto T4 Bright Field Cell Counter (Nexcelom, Lawrence, MA). Cell viability was determined by counting viable cells followed by statistical analysis.

VEGF ELISA. Human Müller cells (MIO-M1) were treated with Example 17 (A229) for 4 hours and then co-incubated with cobalt chloride (CoCl₂, 100 μM). Aliquots of the cell culture supernates were removed, assayed for the VEGF. The levels of human VEGF (hVEGF) were measured using DuoSet ELISA Development kits (R&D Systems Inc., Minneapolis, MN, USA) according to the manufacturer's protocol.

ROS measurement. 661 W cells (mouse cone precursor cell line) were treated with 25 μM of Example 17 (A229) or FA for 4 hours, then 150 μM palmitate (PA) was added and co-incubated with the cells for another 24 hours. H2DCFDA within cells was oxidized to fluorescent dichlorofluorescein (DCF), allowing us to monitor intracellular oxidation status. The DCF fluorescence signal was measured by Wallac Victor 1420 microplate reader. The DCF readout was normalized by the protein concentration in each well.

| | EC₅₀ | Maximal relative | Maximal relative |
|---|---|---|---|
| | hPPARα | activation PPARb/d | activation |
| Compound | (μM) ± SEM | (% ± SEM) | PPARg (% ± SEM) |
| 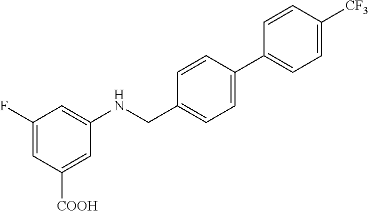 [SMILES: O=C(O)c1cc(F)cc(NCc2ccc(-c3ccc(C(F)(F)F)cc3)cc2)c1] | 0.083 ± 0.011 | 31.1 ± 1.0 | 22.5 ± 6.7 |
| 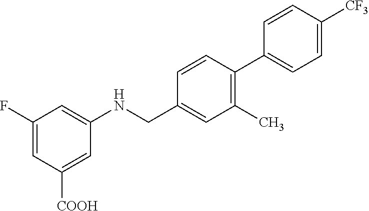 [SMILES: Cc1cc(CNc2cc(F)cc(C(=O)O)c2)ccc1-c1ccc(C(F)(F)F)cc1] | 0.080 ± 0.032 | 25.1 ± 4.1 | 35.7 ± 4.2 |
| 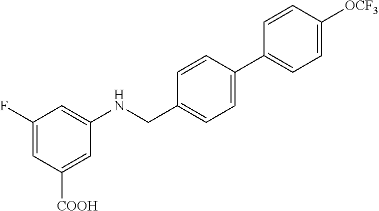 [SMILES: O=C(O)c1cc(F)cc(NCc2ccc(-c3ccc(OC(F)(F)F)cc3)cc2)c1] | 0.064 ± 0.018 | 6.1 ± 3.6 | 9.3 ± 2.3 |
| 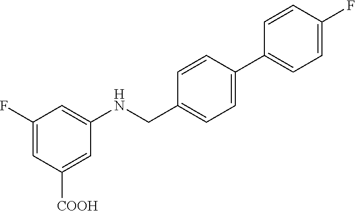 [SMILES: O=C(O)c1cc(F)cc(NCc2ccc(-c3ccc(F)cc3)cc2)c1] | 0.59 ± 0.051 | 12.1 ± 1.0 | 11.2 ± 3.0 |
| 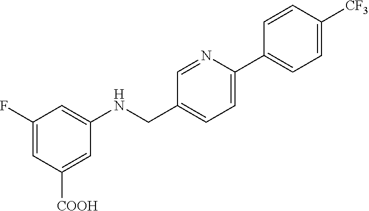 [SMILES: O=C(O)c1cc(F)cc(NCc2ccc(-c3ccc(C(F)(F)F)cc3)nc2)c1] | 0.30 ± 0.096 | 21.1 ± 6.2 | 19.9 ± 10.3 |

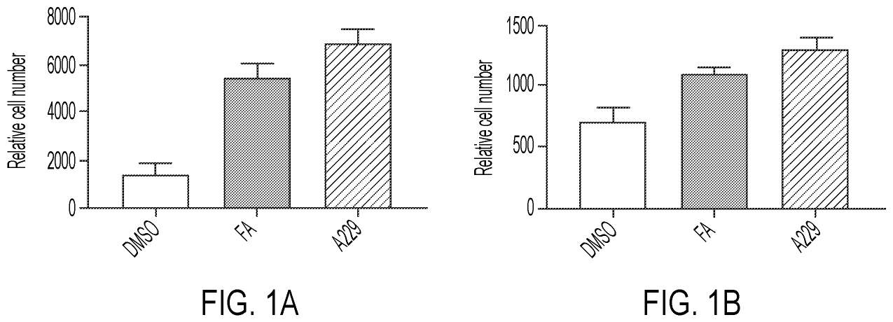

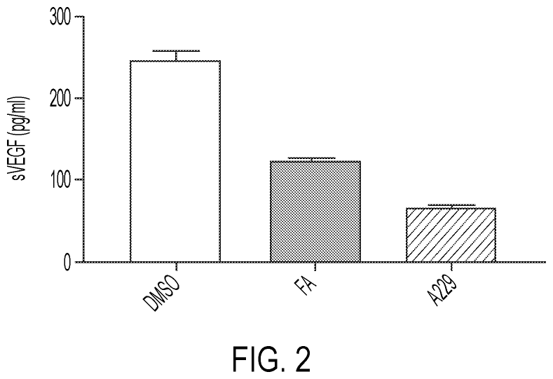

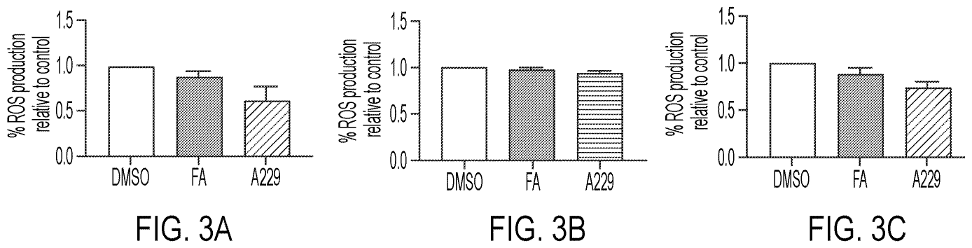

To confirm PPARα agonism, the compound of Example 17 (A229) was assessed in a variety of cell-based phenotypic assays, established to provide outputs indicative of PPARα agonism. These studies revealed that at 25 μM Example 17: 1) exhibited cell protection upon palmitate challenge in ARPE19, a cell line derived from human RPE, 661 W, a murine photoreceptor precursor cell line and MIO-M1 cells, a cell line derived from human Müller cells (FIG. 1), 2) reduced VEGF secretion in MIO-M1 and ARPE19 cells following hypoxic challenge induced by CoCl₂ (FIG. 2), and 3) reduced the production of reactive oxygen species (ROS) in 661 W, primary human RPE (hRPE), and MIO-M1 cells following palmitate challenge (FIG. 3).

### Example 50. Cell-Model Assessment for Dual Modulation

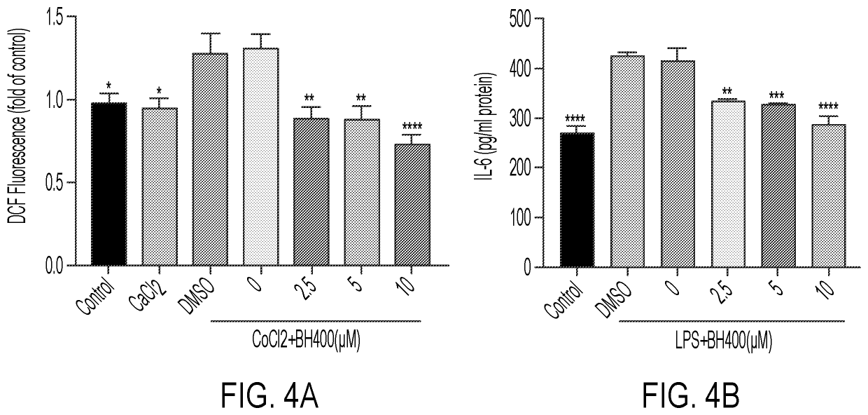

With the luciferase data suggesting dual target modulation, dual modulation and potential benefits thereof in more disease relevant cell-based models was investigated. A known indicator of PPARα agonism is a reduction in oxidative stress following CoCl₂ treatment. This is a cell model of hypoxia, in which the exposure of cells to CoCl₂ results in oxidative stress, which is ameliorated by mitochondrial protection induced through PPARα agonism. Pretreatment of HMC3 cells (microglia) with Example 31 shows a dose-dependent protection against CoCl₂-induced ROS generation (FIG. 4A). Characterization of the STING inhibitory activity of Example 31 is complicated by crosstalk between the PPARα and STING signaling pathways. Direct activity from STING inhibition is often characterized by the detection of IL-6 secretion. Although STING directly regulates IL-6 expression, some crosstalk between PPARα and NF-κB may also affect IL-6 signaling, although this relationship has yet to be fully elucidated (Ma, X., et al., Proc Natl Acad Sci USA, 2022, 119 (48), e2208934119; and Hong, C., et al., Nature, 2022, 607 (7918), 366-373). As shown in FIG. 4B, Example 31 protects against lipopolysaccharide-induced IL-6 secretion in a dose-dependent manner in HMC3 cells.

### Example 51. Cytoprotective Effects

The cytoprotective effects of SN011, Example 17 (A229), and Example 31 in different cell types were examined.

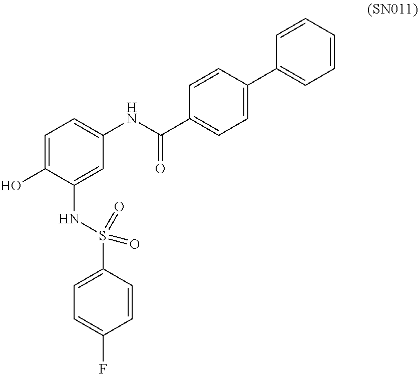 [SMILES: O=C(Nc1ccc(O)c(NS(=O)(=O)c2ccc(F)cc2)c1)c1ccc(-c2ccccc2)cc1]

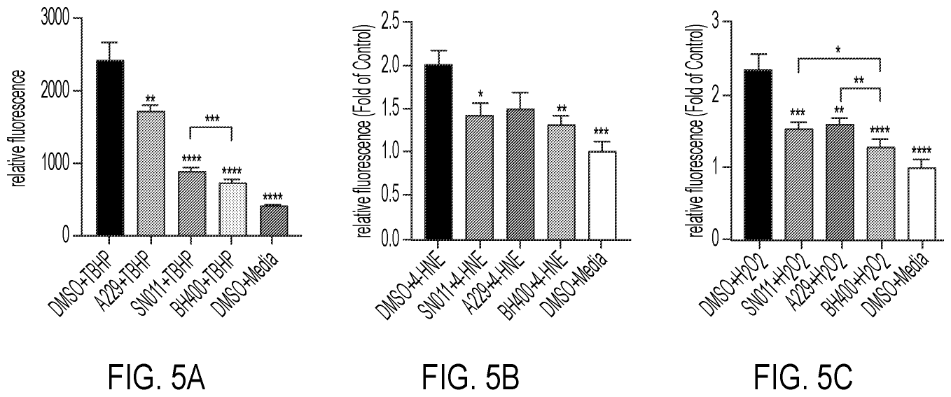

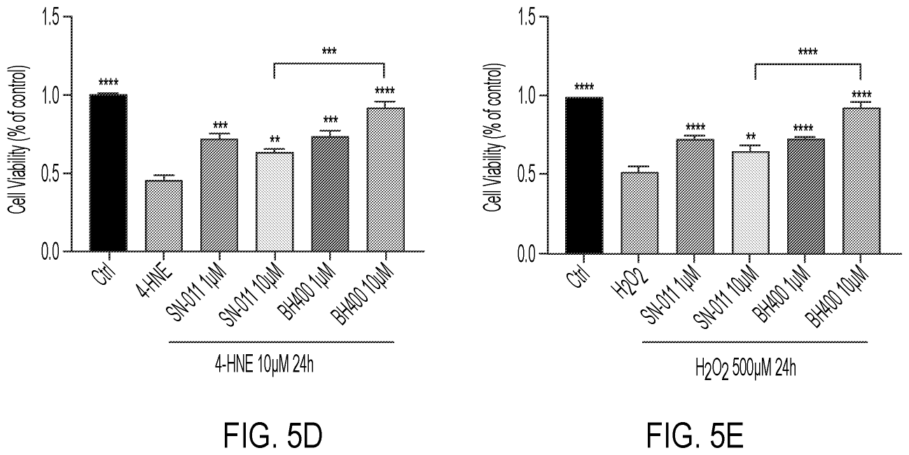

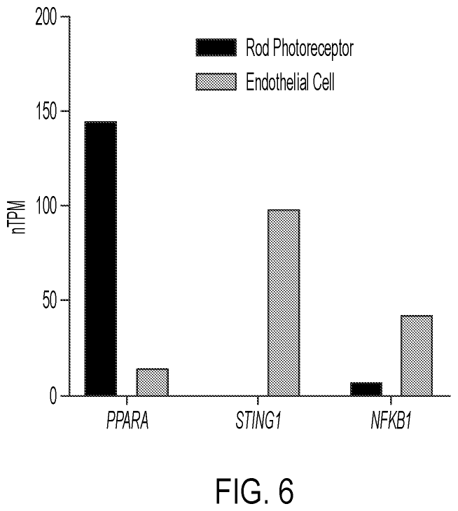

In HMC3 microglia, STING inhibition (SN-011) shows a greater reduction in oxidative stress than PPARα agonism (Example 31 (BH400), FIG. 5A). This may be due to immune cells exhibiting lower expression of PPARα and higher expression of STING (FIG. 6), leading to more STING-driven oxidative stress and thus a more pronounced protection from STING inhibition over selective PPARα agonism. In fact, treatment with Example 31 elicits a statistically significant reduction compared to selective PPARα (Example 17 (A229)) or STING (SN-011) modulation alone, indicating additive value in modulating both targets. It is worth noting that SN-011 exhibits a STING IC₅₀ of 76 nM in the THP-1 luciferase assay, nearly 100-fold more potent than Example 31.

For these studies, 12.5 μM of Example 31 (BH400) was utilized, a concentration only 1.5× the STING IC₅₀ determined in the luciferase assay, further illustrating additivity in dual modulation. In 661 W photoreceptor cells (FIGS. 5B/C), the difference between SN011 and Example 17 is less pronounced, likely due to higher PPARα expression in these cells and the ability of PPARα agonists to indirectly attenuate STING signaling (Ma, X., et al., Proc Natl Acad Sci USA 2022, 119 (48), e2208934119). Dual modulation in this cell type presents a statistically significant improvement over single-target modulation in H₂O₂ but not 4-HNE-induced oxidative stress.

Viability of 661 W cells after treatment with 4-HNE or H₂O₂ exhibited no significant difference at 1 μM of either SN-011 or Example 31. However, at 10 μM, Example 31 significantly improved viability compared to SN-011, once again suggesting a benefit for dual modulation (FIG. 5D/E). As the 1 μM concentration is 8.4× lower than the STING IC₅₀ and 20% lower than the PPARα EC₅₀ of Example 31, improvement in cell viability was unexpected and could be explained by cell type variability between 661 W cells and the THP-1 cells used for luciferase activity. Although Example 31 shows continued improvement in viability from 1 μM to 10 μM, it remains unclear whether PPARα or STING signaling is more important for driving activity between these two concentrations. In contrast, SN-011 fails to show a dose-response improvement between 1 μM and 10 μM. The inability for further STING inhibition to drive improvements in neuronal cell viability reinforces the premise for dual modulation.

Example 52. The following illustrate representative pharmaceutical dosage forms, containing a compound of formula (I) (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 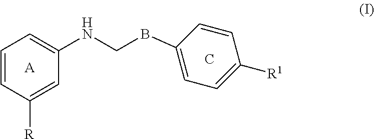
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
B is phenyl, a 5-membered heteroaryl, or a 6-membered heteroaryl;
R¹ is H, halo, cyano, (C₁-C₄)alkyl, (C₃-C₆)cycloalkyl, (C₁-C₄)alkylsulfonyl, hydroxy, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

2. The compound or salt of claim 1, which is a compound of formula (Ia): 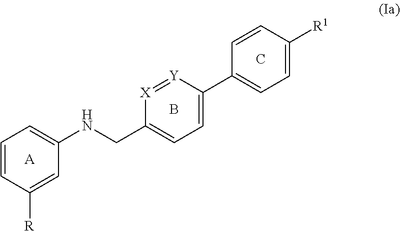
or a salt thereof, wherein:
R is —COOR², —CN, or 5-tetrazolyl;
X and Y are both CH; or one of X and Y is CH and the other is N.
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

3. The compound or salt of claim 1, which is a compound of formula (Ib): 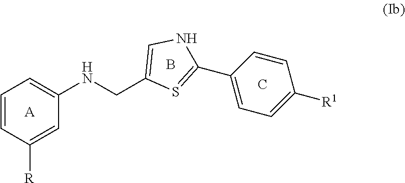
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

4. The compound or salt of claim 1, which is a compound of formula (Ic): 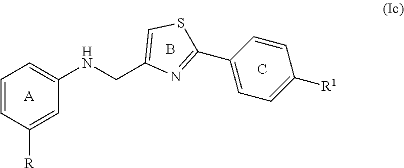
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

5. The compound or salt of claim 1, which is a compound of formula (Id): 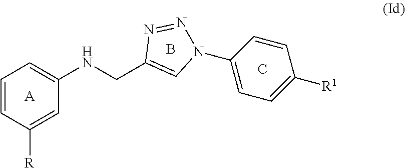
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

6. The compound or salt of claim 1, which is a compound of formula (Ie): 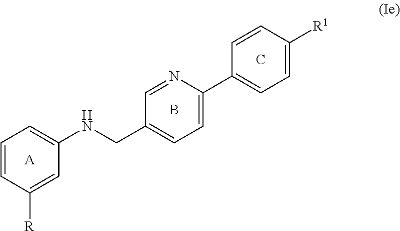
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

7. The compound or salt of claim 1, which is a compound of formula (If): 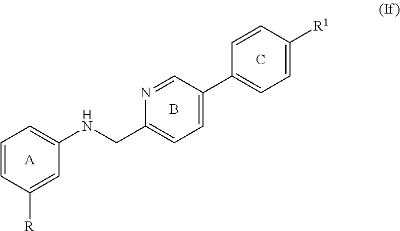
or a salt thereof, wherein:
R is —COOR², —CN, or 5-tetrazolyl;
ring A is additionally optionally substituted with one or more groups independently selected from the group consisting of halo, hydroxy, (C₁-C₄)alkyl, (C₁-C₆)alkoxy, and NR^{a}R^{b};
R¹ is H, halo, cyano, (C₁-C₄)alkyl, (C₃-C₆)cycloalkyl, or (C₁-C₄)alkoxy, which (C₁-C₄)alkyl, (C₃-C₆)cycloalkyl, and (C₁-C₄)alkoxy is optionally substituted with one or more groups independently selected from halo;
R² is H or (C₁-C₄)alkyl;
ring B is additionally optionally substituted with one or more groups R^{x} that are independently selected from the group consisting of (C₁-C₄)alkyl, hydroxy, (C₁-C₄)alkoxy, nitro, and halo, wherein any (C₁-C₄)alkyl, and (C₁-C₄)alkoxy is optionally substituted with one or more groups independently selected from halo;
ring C is additionally optionally substituted with one or more groups R^{y} that are that are independently selected from the group consisting of (C₁-C₄)alkyl, hydroxy, (C₁-C₄)alkoxy, nitro, NR^{c}R^{d}; and halo, wherein any (C₁-C₄)alkyl, and (C₁-C₄)alkoxy is optionally substituted with one or more groups independently selected from halo;
each R^{a} and R^{b} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{a} and R^{b} together with the nitrogen to which they are attached form a aziridino, azetidino, morpholino, piperazino, pyrrolidino or piperidino; and
each R^{c} and R^{d} is independently selected from the group consisting of H, (C₁-C₄)alkyl, (C₃-C₆)cycloalkyl, (C₃-C₆)cycloalkyl(C₁-4₆)alkyl, and (C₁-C₄)alkanolyl, or R^{c} and R^{d} together with the nitrogen to which they are attached form a aziridino, azetidino, morpholino, piperazino, pyrrolidino or piperidino.

8. The compound of any one of claims 1-7, wherein R is —COOR².

9. The compound of any one of claims 1-7, wherein R is —CN.

10. The compound of any one of claims 1-7, wherein R is 5-tetrazolyl.

11. The compound or salt of claim 1, B is phenyl that is additionally optionally substituted with one or more groups R^{y} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

12. The compound or salt of claim 1, B is a 5-membered heteroaryl that is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

13. The compound or salt of claim 1, B is a 6-membered heteroaryl that is additionally optionally substituted with one or more groups R^{x} that are independently selected from the group consisting of (C₁-C₄)alkyl, hydroxy, (C₁-C₄)alkoxy, and halo, wherein any (C₁-C₄)alkyl, and (C₁-C₄)alkoxy is optionally substituted with one or more groups independently selected from halo.

14. The compound or salt of any one of claims 1-10, wherein ring A is additionally optionally substituted with one or more groups independently selected from the group consisting of halo, hydroxy, and (C₁-C₆)alkoxy.

15. The compound or salt of any one of claims 1-7, wherein ring A is additionally optionally substituted with one or more groups independently selected from the group consisting of halo.

16. The compound or salt of any one of claims 1-8, wherein ring A is:

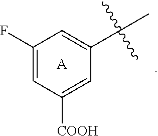

17. The compound or salt of any one of claims 1-8, wherein ring A is additionally optionally substituted with hydroxy.

18. The compound or salt of any one of claims 1-8, wherein ring A is:

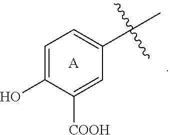

19. The compound or salt of claim 2, wherein X and Y are both CH.

20. The compound or salt of claim 2, wherein X is CH and Y is N.

21. The compound or salt of claim 2, wherein X is N and Y is CH.

22. The compound or salt of any one of claims 1-21, wherein ring B is substituted with one or more groups R^{x} that are independently selected from the group consisting methoxy, methyl, hydroxy, and chloro.

23. The compound or salt of any one of claims 1-22, wherein R¹ is fluoro, chloro, methyl, trifluoromethyl, methoxy, trifluoromethoxy, cyano, or cyclopentyl.

24. The compound or salt of any one of claims 1-23, wherein ring C is not optionally substituted with one or more groups R^{y}.

25. The compound or salt of any one of claims 1-8 and 11-24, wherein R² is H or (C₁-C₄)alkyl.

26. The compound or salt of any one of claims 1-8, 11-15, 17, and 19-24, wherein R² is (C₁-C₄)alkyl.

27. The compound or salt of any one of claims 1-8, 11-15, 17, and 19-24, wherein R² is methyl.

28. A pharmaceutical composition comprising a compound of formula (I) as described in any one of claims 1-27 or a pharmaceutically acceptable salt thereof and a pharmaceutically acceptable carrier.

29. A method for treating diabetic retinopathy in an animal, comprising administering to the animal, a compound of formula (I) as described in any one of claims 1-27 or a pharmaceutically acceptable salt thereof.

30. A method for agonizing the activity of a peroxisome proliferator-activated receptor alpha comprising contacting the receptor with a compound of formula (I) as described in any one of claims 1-27 or a salt thereof in vitro or in vivo.

31. A method for agonizing the activity of a peroxisome proliferator-activated receptor alpha in an animal comprising administering to the animal, a compound of formula (I) as described in any one of claims 1-27 or a pharmaceutically acceptable salt thereof.

32. A method for treating pain in an animal comprising administering to the animal, a compound of formula (I) as described in any one of claims 1-27 or a pharmaceutically acceptable salt thereof.

33. A method for treating a disease or condition associated with the liver in an animal (e.g., fibrosis, nonalcoholic fatty liver disease (NAFLD or metabolic dysfunctio-associated steatotic liver disease MASLD), or nonalcoholic steatohepatitis (NASH)), comprising administering to the animal, a compound of formula (I) as described in any one of claims 1-27 or a pharmaceutically acceptable salt thereof.

34. A compound of formula (I) as described in any one of claims 1-27 or a pharmaceutically acceptable salt thereof for use in medical therapy.

35. A compound of formula (I) as described in any one of claims 1-27 or a pharmaceutically acceptable salt thereof for the prophylactic or therapeutic treatment of diabetic retinopathy.

36. A compound of formula (I) as described in any one of claims 1-27 or a pharmaceutically acceptable salt thereof for agonizing the activity of a peroxisome proliferator-activated receptor alpha.

37. A compound of formula (I) as described in any one of claims 1-27 or a pharmaceutically acceptable salt thereof for the prophylactic or therapeutic treatment of pain.

38. A compound of formula (I) as described in any one of claims 1-27 or a pharmaceutically acceptable salt thereof for the prophylactic or therapeutic treatment of a disease or condition associated with the liver (e.g., fibrosis, nonalcoholic fatty liver disease (NAFLD or metabolic dysfunctio-associated steatotic liver disease MASLD), or nonalcoholic steatohepatitis (NASH)).

39. The use of a compound of formula (I) as described in any one of claims 1-27 or a pharmaceutically acceptable salt thereof to prepare a medicament for treating diabetic retinopathy in an animal.

40. The use of a compound of formula (I) as described in any one of claims 1-27 or a pharmaceutically acceptable salt thereof to prepare a medicament for agonizing the activity of a peroxisome proliferator-activated receptor alpha in an animal.

41. The use of a compound of formula (I) as described in any one of claims 1-27 or a pharmaceutically acceptable salt thereof to prepare a medicament for treating pain in an animal.

42. The use of a compound of formula (I) as described in any one of claims 1-27 or a pharmaceutically acceptable salt thereof to prepare a medicament for treating a disease or condition associated with the liver (e.g., fibrosis, nonalcoholic fatty liver disease (NAFLD or metabolic dysfunctio-associated steatotic liver disease MASLD), or nonalcoholic steatohepatitis (NASH)) in an animal.

43. A compound or salt that modulates the activity of PPARα and STING.

44. A compound or salt that has agonist activity of PPAR-alpha of less than 5 μM and that inhibits STING at less than 10 μM.

45. The compound of claim 43 or 44, which is a compound of formula (I) as described in claim 1 or a salt thereof.

46. The compound of claim 43 or 44, which is a compound of formula (I) as described in claim 1 or a salt thereof, wherein Ring A has the following structure:

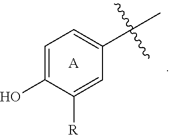

47. A compound selected from: 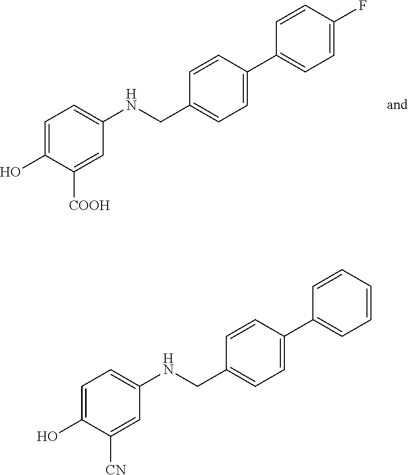 [SMILES: O=C(O)c1cc(NCc2ccc(-c3ccc(F)cc3)cc2)ccc1O ; N#Cc1cc(NCc2ccc(-c3ccccc3)cc2)ccc1O]
or a salt thereof.

48. A method for treating a disease in an animal where both PPARα and STING are implicated and modulation of both targets may be beneficial, comprising administering to the animal, a compound or salt that is a dual modulator of PPARα and STING.

49. A method for treating a disease in an animal where agonism of PPAR-alpha and inhibition of STING are both implicated, comprising administering to the animal, a compound or pharmaceutically acceptable salt that is an agonist of PPARα and an inhibitor of STING.

50. The method of claim 46 or 47, wherein a compound of formula (I) as described in claim 1 or a pharmaceutically acceptable salt thereof, wherein Ring A has the following structure:

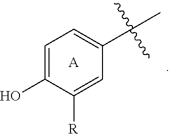

51. The method of claim 46 or 47, wherein the compound or the pharmaceutically acceptable salt is selected from: 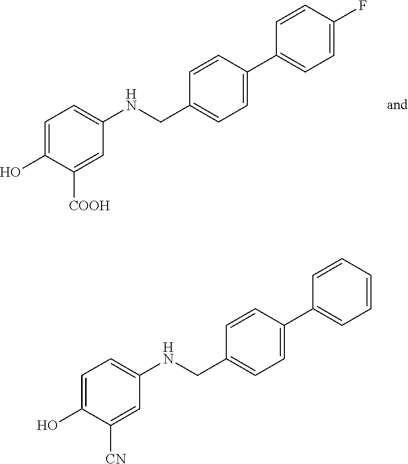 [SMILES: O=C(O)c1cc(NCc2ccc(-c3ccc(F)cc3)cc2)ccc1O ; N#Cc1cc(NCc2ccc(-c3ccccc3)cc2)ccc1O]
and salts thereof.

52. The method of any one of claims 48-51, wherein the disease is an inflammatory retinal disease, pain, or a liver disease (e.g., fibrosis, NASH or NAFLD or metabolic dysfunctio-associated steatotic liver disease MASLD).

53. The method of any one of claims 48-51, wherein the disease is diabetic retinopathy (DR) or age-related macular degeneration (AMD).

54. A compound or salt that modulates the activity of PPARα and STING for use in medical therapy.

55. A compound or salt that modulates the activity of PPARα and STING for the prophylactic or therapeutic treatment of a disease or condition where both PPARα and STING are implicated and modulation of both PPARα and STING may be beneficial.

56. A compound or salt that has dual activity as an agonist of PPAR-alpha and as an inhibitor of STING for the prophylactic or therapeutic treatment of a disease or condition wherein agonism of PPAR-alpha and as an inhibition of STING may be beneficial.

57. The compound or salt of claim 55 or 56, which is a compound of formula (I) as described in claim 1 or a pharmaceutically acceptable salt thereof, wherein Ring A has the following structure:

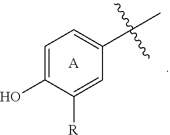

58. The compound or salt of claim 55 or 56, which is selected from: 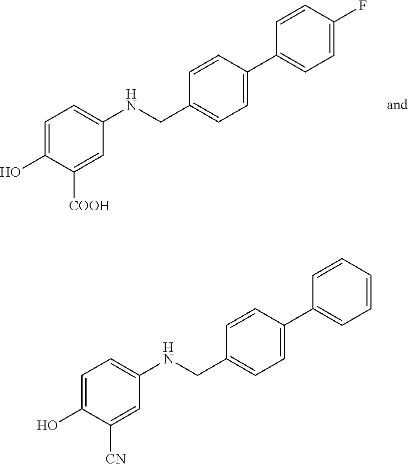 [SMILES: O=C(O)c1cc(NCc2ccc(-c3ccc(F)cc3)cc2)ccc1O ; N#Cc1cc(NCc2ccc(-c3ccccc3)cc2)ccc1O]
and salts thereof.

59. The compound or salt of any one of claims claim 53-56, wherein the disease or condition is an inflammatory retinal disease, pain, or a liver disease (e.g., fibrosis, NASH or NAFLD or metabolic dysfunctio-associated steatotic liver disease MASLD).

60. The compound or salt of any one of claims claim 53-56, wherein the disease or condition is diabetic retinopathy (DR) or age-related macular degeneration (AMD).

61. The use of a compound or salt that modulates the activity of PPARα and STING to prepare a medicament for treating a disease or condition where both PPARα and STING are implicated and modulation of both PPARα and STING may be beneficial.

62. The use of a compound or salt that has dual activity as an agonist of PPAR-alpha and as an inhibitor of STING to prepare a medicament for treating a disease or condition wherein agonism of PPAR-alpha and as an inhibition of STING may be beneficial.

63. The use of claim 61 or 62, wherein the compound or salt is a compound of formula (I) as described in claim 1 or a pharmaceutically acceptable salt thereof, wherein Ring A has the following structure:

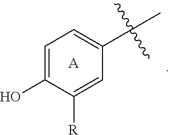

64. The use of claim 61 or 62, wherein the compound or salt is selected from: 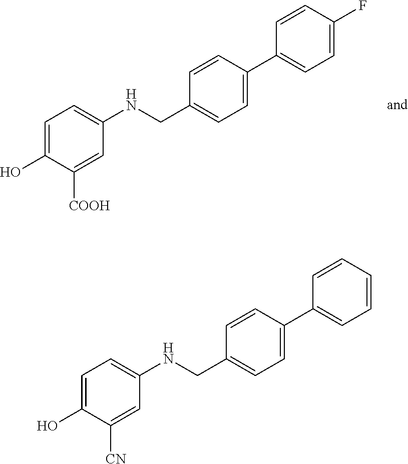 [SMILES: O=C(O)c1cc(NCc2ccc(-c3ccc(F)cc3)cc2)ccc1O ; N#Cc1cc(NCc2ccc(-c3ccccc3)cc2)ccc1O]
and salts thereof.

65. The use of any one of claims 61-64, wherein the disease or condition is an inflammatory retinal disease, pain, or a liver disease (e.g., fibrosis, NASH, NAFLD or metabolic dysfunctio-associated steatotic liver disease MASLD).

66. The use of any one of claims 61-64, wherein the disease or condition is diabetic retinopathy (DR) or age-related macular degeneration (AMD).
